(19) United States
(12) Patent Application Publication
(10) Pub. No.: US 2025/0003979 A1
(43) Pub. Date: Jan. 2, 2025

(54) METABOLOMIC PROFILES FOR PREDICTION OF FUNCTIONAL NEUROLOGICAL OUTCOME OR DEATH FOLLOWING SEVERE TRAUMATIC BRAIN INJURY

(71) Applicants: UTI Limited Partnership, Calgary (CA); The Governors of the University of Alberta, Edmonton (CA)

(72) Inventors: Brent WINSTON, Calgary (CA); Mohammad Mehdi BANOEI, Calgary (CA); David WISHART, Edmonton (CA)

(21) Appl. No.: 18/576,867

(22) PCT Filed: Jul. 11, 2022
(86) PCT No.: PCT/CA2022/051078

§ 371 Date: Jan. 5, 2024

Related U.S. Application Data
(60) Provisional application No. 63/220,248, filed on Jul. 9, 2021.

(51) Int. Cl. G01N 33/68; G01N 33/53; G01N 33/66

(52) U.S. Cl. CPC G01N 33/6848; G01N 33/5308; G01N 33/66; G01N 33/6806; G01N 2570/00; G01N 2800/2871; G01N 2800/56

(57) ABSTRACT

The present disclosure provides a method of determining a likelihood of a favorable or unfavorable outcome, such as death or a Glasgow Outcome Scale Extended (GOSE) score≤4, in a subject having severe traumatic brain injury (sTBI). The method involves quantitative assessment of multiple metabolites shortly after the injury, such as on day 1 and/or day 4 for changes indicative of outcome. Quantitative mass spectrometry (MS) or proton (¹H) nuclear magnetic resonance spectroscopy (NMR) may be used to assess multiple metabolites within a single blood sample for comparison with a control.

### CROSS REFERENCE TO RELATED APPLICATION

The present application claims benefit of and claims priority to U.S. Provisional Patent Application No. 63/220,248 filed Jul. 9, 2021, the entirety of which is hereby incorporated by reference.

### FIELD

The present disclosure relates generally to metabolomic profiles in serum predict global functional neurological outcome and death at 3 and 12 months following severe traumatic brain injury.

### BACKGROUND

Traumatic brain injury (TBI) is defined as a neurologic injury resulting from an external mechanical force and is one of the most common causes of long-term neurological disability and death¹. Worldwide, approximately 69 million people suffer mild, moderate, and severe TBI annually². There are 5.3 and 7.7 million individuals living with TBI-related disability in US and European Union countries, respectively¹. Severe TBI (sTBI) has a mortality of 30-50% and 30% of survivors have severe neurologic sequelae³⁻⁷. Large variability in the mechanisms of TBI, patterns of brain injury and a large range of outcomes make it difficult to determine prognosis in the first few days following sTBI 8. Clinical factors and neuroimaging findings are not clinically reliable predictors for the prognosis of sTBI outcome^{9,10}. More accurate blood biomarkers are urgently needed to help inform discussions with surrogate decision-makers about the level of care, while the patient is comatose, and to help plan rehabilitation and support services. Metabolomics is widely used to provide potential insights into mechanisms of injury and may allow the development of very sensitive and specific biomarkers for the prognosis of TBI¹¹.

### SUMMARY

There is provided herein a method of determining a likelihood of an unfavourable outcome in a subject having a severe traumatic brain injury (sTBI), comprising: obtaining a sample from a subject at day 1 and/or day 4 post sTBI, measuring an amount of a plurality of metabolites in the sample, and comparing levels of the plurality of metabolites in the sample with a control; wherein an outcome of the comparing step is an increase or decrease in quantity of said plurality of metabolites in the sample relative to the control, and wherein unfavourable outcome is determined as likely when: in the day 1 sample, at least two, or at least 5, of the following metabolites of the plurality of metabolites are changed relative to the control: C3:1, LYSOC17:0, LYSOC18:0, LYSOC16:0, C18:2, LYSOC18:1, C14, C18:1, C18, C16, tyrosine, homocysteine, C3, CO, C4, ornithine, LYSOC14:0, SM 16:1 OH, LYSOC20:3, LYSOC28:1, phenylalanine, glutamine, PC ae 36:0, histidine, SM 20:2, isoleucine, citrulline, methionine-sulfoxide, asymmetric dimethylarginine, C5OH, C10:2, acetyl-ornithine, C9, methionine-sulfoxide, spermine, serotonin, serine, trans-hydroxyproline, succinate, gluconate, acetone, lactate, glycerol, betaine, choline, alanine, and 3-hyroxyisovalerate; and/or in the day 4 sample, at least two, or at least 5, of the following metabolites of the plurality of metabolites are changed relative to the control: valine, N-acetylaspartate, tyrosine, lysine, dimethylsulfone, taurine, gluconate, hypoxanthine, beta-alanine, C3OH, glutamic acid, LYSOC18:0, PC36:0AA, C18:2, C3:1, C3, ornithine, CO, SM 16:1 OH, LYSOC14:0, LYSOC20:3, homocysteine, C16:1, glutamine, beta-hydroxybutyric acid, uric acid, serotonin, C9, PC ae 36:0, methionine-sulfoxide, serine, a-dimethylarginine, spermine, and trans-hydroxyproline; wherein the unfavourable outcome comprises a Glasgow Outcome Scale Extended (GOSE) score≤4 from 3 months to 12 months post sTBI. For example, the likelihood of unfavorable outcome may be determined for three months following the sTBI, or may be determined for twelve months following the sTBI.

According to an embodiment described herein, the likelihood of the unfavorable outcome may be determined from a Day 1 sample, with an increase or decrease in the at least two, or the at least 5, metabolites as per Tables 35A, 35B, 37A, or 37B.

In another embodiment, the likelihood of the unfavorable outcome may be determined from a Day 4 sample, with an increase or decrease in the at least two, or the at least 5, metabolites as per Tables 36A, 36B, 38A, or 38B.

Further, there is described herein a method of determining a likelihood of mortality within three months in a subject having a severe traumatic brain injury (sTBI), comprising: obtaining a sample from a subject at day 1 and/or day 4 post sTBI, measuring an amount of a plurality of metabolites in the sample, and comparing levels of the plurality of metabolites in the sample with a control, wherein an outcome of the comparing step is an increase or decrease in quantity of said plurality of metabolites in the sample relative to the control, and wherein increased likelihood of mortality is determined when: in the day 1 sample, at least two, or at least 5, of the following metabolites of the plurality of metabolites are changed relative to the control: C3:1, PC aa 38:0, glucose, C16:2, leucine, C10:2, valine, isoleucine, histidine, C16OH, glutamine, betaine, 3-hydroxyisovalerate, citrate, and lactate;
and/or in the day 4 sample, at least two, or at least 5, of the following metabolites of the plurality of metabolites are changed relative to the control: isobutyrate, valine, lysine, 2-aminobutyrate, hypoxanthine, taurine, gluconate, betaine, alpha-ketoglutaric acid, C16:2OH, hippuric acid, indole acetic acid, PC aa 36:0, ornithine, PC aa 38:0, alpha-aminoadipic acid, tryptophan, leucine, C12:1, C6, glutamine, and LysoPC 26:0.

Optionally, the likelihood of mortality may be determined from a Day 1 sample, with increase or decrease in said at least 2, or said at least 5, of the plurality of metabolites as per Tables 43A, 43B, 45A, or 45B.

Further, the likelihood of mortality may be determined from a Day 4 sample, with an increase or decrease in the at least 2, or the at least 5, of the plurality of metabolites as per Tables 44A, 44B, 46A, 46B, or 46C.

An embodiment is described herein of a method of determining a likelihood of an outcome in a subject having a severe traumatic brain injury (sTBI) or suspected of having an sTBI, comprising: obtaining a sample from a subject at day 1 or day 4 post-sTBI, measuring an amount of a plurality of metabolites in the sample, and comparing levels of the plurality of metabolites in the sample with a control, wherein an outcome of the comparing step is an increase or decrease in quantity of said plurality of metabolites in the sample, (i) wherein the likelihood of unfavourable outcome at 3 months is determined: (a1) in a day 1 sample assessed by quantitative MS, wherein at least 2, at least 5, or at least 10 metabolites of said plurality of metabolites are increased or decreased relative to the control as indicated of the 26 metabolites in Table 35A or of the 13 metabolites in Table 35B; or (a2) in a day 4 sample assessed by MS/MS, wherein at least 2, at least 5, or at least 10 metabolites of said plurality of metabolites are increased or decreased relative to the control as indicated, of the 15 metabolites in Table 36A, or of the 11 metabolites in Table 36B; or (b1) in a day 1 sample assessed by proton (1H) nuclear magnetic resonance spectroscopy (NMR), wherein at least 2, at least 5, or at least 10 metabolites of said plurality of metabolites are increased or decreased relative to the control as indicated, of the 12 metabolites in Table 39A, or at least 5 of the 6 metabolites in Table 39B; or (b2) in a day 4 sample assessed by NMR, wherein at least 2, or at least 5 metabolites of said plurality of metabolites are increased or decreased relative to the control as indicated, of the 9 metabolites in Table 40A, or of the 6 metabolites in Table 40B; or (ii) wherein the likelihood of unfavourable outcome at 12 months is determined: (c1) in a day 1 sample, assessed by QUANTITATIVE MS, wherein at least 2, at least 5, or at least 10 metabolites of said plurality of metabolites are increased or decreased relative to the control as indicated, of the 21 metabolites in Table 37A or of the 15 metabolites in Table 37B; or (c2) in a day 4 sample, assessed by quantitative MS, wherein at least 2, at least 5, or at least 10 metabolites of said plurality of metabolites are increased or decreased relative to the control as indicated, of the 18 metabolites in Table 38A or of the 13 metabolites in Table 38B; or (d1) in a day 1 sample assessed by NMR, wherein at least 2, or at least 5 metabolites of said plurality of metabolites are increased or decreased relative to the control as indicated, of the 8 metabolites in Table 41A, or at least 4 of the 5 metabolites in Table 41B; or (d2) in a day 4 sample assessed by NMR, wherein at least 2, or at least 5 metabolites of said plurality of metabolites are increased or decreased relative to the control as indicated, of the 9 metabolites in Table 42A, or of the 5 metabolites in Table 42B; or (iii) wherein the likelihood of mortality outcome is determined: (e1) in a day 1 sample, assessed by quantitative MS, wherein at least 2, at least 5, or at least 10 metabolites of said plurality of metabolites are increased or decreased relative to the control as indicated, of the 19 metabolites in Table 43A or of the 11 metabolites in Table 43B; or (e2) in a day 4 sample, assessed by quantitative MS, wherein at least 2, at least 5, or at least 10 metabolites of said plurality of metabolites are increased or decreased relative to the control as indicated, of the 22 metabolites in Table 44A, or of the 16 metabolites in Table 44B; or (f1) in a day 1 sample, assessed by NMR, wherein at least 2, at least 5, or at least 10 metabolites of said plurality of metabolites are increased or decreased relative to the control as indicated, of the 17 metabolites in Table 45A, or at least 4 of the 5 metabolites in Table 45B; or (f2) in a day 4 sample, assessed by NMR, wherein at least 2, or at least 5 metabolites of said plurality of metabolites are increased or decreased relative to the control as indicated, of the 16 metabolites in Table 46A, of the 8 metabolites in Table 46B, or at least 2 or 4 of the 5 metabolites in Table 46C.

The likelihood of unfavorable outcome may be predicted for three months following the sTBI, or may be predicted for twelve months following the sTBI. Further, the method may be used for a prediction of likelihood of mortality by 3 months.

The method of any one of claims 1 to 12, wherein sample is a serum sample, or may be from blood, plasma, or tissue.

The control may be a value determined from individuals with an orthopedic injury (OI) without head injury, or from a cohort of individuals with sTBI having a favorable outcome, or other suitable control.

The method can assess the amount of the plurality of metabolites are assessed by NMR, or by a quantitative MS platform, or any other suitable platform or technology capable of assessing such metabolites.

Samples obtained between days 1 and 5 post TBI are preferred, but later samples may be utilized. For example, the sample may be obtained at day 1 following the sTBI, or may be obtained at day 4 following the sTBI. If multiple samples are obtained, for example at both days 1 and 4, these may both be utilized in the method described herein.

According to an embodiment described herein, the likelihood of unfavourable outcome at 3 months may determined by assessing the increase or decrease in the following metabolites: (a1) in a day 1 sample, assessed by quantitative MS wherein the metabolites are increased or decreased as indicated in Table 35A or Table 35B; (a2) in a day 4 sample, assessed by quantitative MS wherein the metabolites are increased or decreased as indicated in Table 36A or Table 36B; (b1) in a day 1 sample, assessed by NMR wherein the metabolites are increased or decreased as indicated in Table 39A or Table 39B; or (b2) in a day 4 sample, assessed by NMR wherein the metabolites are increased or decreased as indicated in Table 40A or Table 40B.

According to an embodiment described herein, the likelihood of unfavourable outcome at 12 months may be determined by assessing the increase or decrease in the following metabolites: (c1) in a day 1 sample, assessed by quantitative MS wherein the metabolites are increased or decreased as indicated in Table 37A or Table 37B; (c2) in a day 4 sample, assessed by quantitative MS wherein the metabolites are increased or decreased as indicated in Table 38A or Table 38B; (d1) in a day 1 sample, assessed by NMR wherein the metabolites are increased or decreased as indicated in Table 41A or Table 41B; or (d2) in a day 4 sample, assessed by NMR wherein the metabolites are increased or decreased as indicated in Table 42A or Table 42B.

Further, according to an embodiment described herein, the likelihood of mortality outcome at 3 months may be determined by assessing the increase or decrease in the following metabolites: (e1) in a day 1 sample, assessed by quantitative MS wherein the metabolites are increased or decreased as indicated in Table 43A or Table 43B; (e2) in a day 4 sample, assessed by quantitative MS wherein the metabolites are increased or decreased as indicated in Table 44A or Table 44B; (f1) in a day 1 sample, assessed by NMR wherein the metabolites are increased or decreased as indicated in Table 45A or Table 45B; or (f2) in a day 4 sample, assessed by NMR wherein the metabolites are increased or decreased as indicated in Table 46A, Table 46B, or Table 46C.

For example, the likelihood of mortality outcome at 3 months is determined by assessing the increase or decrease in metabolites: (e1) in a day 1 sample, assessed by quantitative MS wherein the metabolites are increased or decreased as indicated in Table 43B; (e2) in a day 4 sample, assessed by quantitative MS wherein the metabolites are increased or decreased as indicated in Table 44B; (f1) in a day 1 sample, assessed by NMR wherein the metabolites are increased or decreased as indicated in Table 45B; or (f2) in a day 4 sample, assessed by NMR wherein the metabolites are increased or decreased as indicated in Table 46C.

There is described herein a kit for predicting outcome of a traumatic brain injury in a subject comprising: reagents for detecting the metabolites listed in any one of Tables 35A, 35B, 36A, 36B, 37A, 37B, 38A, 38B, 39A, 39B, 40A, 40B, 41A, 41B, 42A, 42B, 43A, 43B, 44A, 44B, 45A, 45B, 46A, 46B, and/or 46C, and instructions for conducting any one of the methods described herein.

In a further aspect, there is provided a method of determining a likelihood of an unfavourable outcome in a subject having a severe traumatic brain injury (sTBI), comprising: obtaining a sample from a subject at day 1 and/or day 4 post sTBI, and measuring an amount of a plurality of metabolites in the sample, comparing levels of the plurality of metabolites in the sample with a control, such as a control indicative of favourable outcome; wherein an outcome of the comparing step is an increase or decrease in quantity of said plurality of metabolites in the sample relative to the control, and wherein unfavourable outcome is determined as likely when: in the day 1 sample, two or more of the following metabolites are increased relative to the control: ornithine, alanine, dimethyl sulfone, carnitine, valine, leucine, adipate, α-ketoglutaric acid, homocysteine, and LysoPCs; and two or more of the following metabolites are decreased relative to the control: N-acetylaspartate (NAA), pyruvate, mannose, hydroxyproline, serotonin, dimethylarginine; and/or in the day 4 sample, two or more of the following metabolites are increased relative to the control: dimethyl sulfone, valine, tryptophan, tyrosine, gluconate, urea, uric acid, NAA, ornithine, kynurenine, and alanine; and two or more of the following metabolites are decreased relative to the control: serotonin, spermine, taurine, β-hydroxybutyric acid, and arginine; wherein the unfavourable outcome comprises a Glasgow Outcome Scale Extended (GOSE) score≤4 from 3 months to 12 months post sTBI. For example, the likelihood of said unfavorable outcome may be determined for a time frame that is three months or twelve months following the sTBI.

A method is described herein for determining a likelihood of death within three months in a subject having a severe traumatic brain injury (sTBI), comprising: obtaining a sample from a subject at day 1 and/or day 4 post sTBI, measuring an amount of a plurality of metabolites in the sample, and comparing levels of the plurality of metabolites in the sample with a control, such as a control indicative of survival at 3-months, wherein an outcome of the comparing step is an increase or decrease in quantity of said plurality of metabolites in the sample relative to the control, and wherein increased likelihood of mortality is determined when: in the day 1 sample, a plurality of the following metabolites are increased relative to the control: glucose, betaine, O-phosphocholine, creatine, citrate, dimethyl sulfone acylcarnitines, methyl histidine, α-aminoadipic acid and arginine; and a plurality of the following metabolites are decreased relative to the control: glutamine, histidine, succinate, isoleucine, leucine, and valine, citrulline, homocysteine, and homovanillic acid; and/or in the day 4 sample, a plurality of the following metabolites are increased relative to the control: creatine, isobutyrate, dimethylsulfone, valine, asparagine, tyrosine indoleacetic acid, α-ketoglutaric acid, hippuric acid, acylcarnitines, citric acid, ornithine, threonine, and tryptophan; and a plurality of the following metabolites are decreased relative to the control: betaine, gluconate, taurine, hypoxanthine, urea, serine, and glutamate, glutamine, creatinine, and hexanoylcarnitine (C6).

In a further aspect there is provided a method of determining a likelihood of an outcome in a subject having a sTBI or suspected of having a sTBI, comprising: obtaining a sample from a subject, and measuring an amount of a plurality of metabolites in the sample; wherein an outcome is characterized by a change in the amount of the predictive metabolites as described herein, such as in Table 5, Table 8 or Table 17.

In one example, the sample is a serum sample.

In one example, the amount of said plurality of metabolites is determined by Proton nuclear magnetic resonance (¹H-NMR) spectroscopy or by tandem mass spectrometry (MS/MS), which may be interchangeably referred to herein as quantitative mass spectrometry, such as targeted direct injection tandem mass spectrometry and reverse phase liquid chromatography tandem mass spectrometry (DI/LC-MS/MS).

In one example, obtaining of said sample occurs at about one day or about four days following the TBI.

In other examples, said likelihood of said unfavorable outcome is evaluated at about three months or about 12 months.

In one example, the subject is a human.

In one aspect there is provided a kit for carrying out characterization of CRC disease in a mammal which comprises two or more reference metabolites selected from the listing of predictive metabolites as provided herein such as in Table 5, Table 8 or Table 17, wherein the reference metabolites are individually packed in the kit. A reference metabolite for each of the different metabolites listed in herein may be used. Optionally, reference metabolites in such a kit may be labelled, for example isotopically. Derivatization reagents or labeling reagents may be included in the kit.

### BRIEF DESCRIPTION OF THE DRAWINGS

Embodiments of the present disclosure will now be described, by way of example only, with reference to the attached Figures.

FIG. 1 is a diagram of the patient flow chart showing patient selection at baseline, and patients with measured GOSE outcome at 3 and 12 months.

FIGS. 2A-2E show PLS-DA scatter plots: discrimination models show high predictive (Q2) separation of patients with unfavorable outcome (●) from favorable outcome (▪) based on serum metabolomic profiling on day 4 and GOSE at 3 months, FIG. 2A: DI-MS/MS using 54 metabolites, FIG. 2B: ¹H-NMR using 26 metabolites. The high predictability is visualized by a good separation between the two cohorts and yielding a Q2>0.5. The model metrics for the day 4 DI-MS/MS model and GOSE at 3 months are R²Y=0.81, Q²Y=0.61 and p=5.4×10−5 and for day 4 ¹H-NMR and GOSE at 3-month is R²Y=0.75, Q²Y=0.52 and p=0.006. The metabolic profile on day 4 serum samples analyzed using DI-MS/MS was more predictive (Q²=0.61) than ¹H-NMR (Q²=0.52). GOSE at 12-months, FIG. 2C: DI-MS/MS using only 31 metabolites, FIG. 2D: ¹H-NMR using only 18 metabolites. The metabolic profile on day 4 serum samples analyzed using DI-MS/MS was more predictive (Q²=0.63) than ¹H-NMR (Q2=0.45). Mortality outcome at 3 months: non-survivor (●) vs survivor outcome (▪), FIG. 2E: DI-MS/MS using 31 metabolites, Q²=0.57. FIG. 2F: 1H-NMR using 17 metabolites. Q²=0.44. These Q² values show a high predictability of metabolic profile on day 4 with DI-MS/MS being better than ¹H-NMR to predict mortality at 3-months.

FIG. 3 is a chart illustrating typical patient age distribution of sTBI (Shapiro-Wilk W=0.94312. p=0.00000).

FIG. 4A and FIG. 4B show MS/MS data of prognosis of GOS-E 12 months for poor outcome versus good outcome based on sTBI Day 1 and Day 4 metabolites, respectively.

FIG. 5A and FIG. 5B show NMR data of prognosis of GOS-E 12 months for poor outcome versus good outcome based on sTBI Day 1 and Day 4 metabolites, respectively.

FIG. 6A and FIG. 6B show MS/MS data of prognosis of mortality and vegetative state for GOS-E 1&2 versus GOS-E 3-8 based on sTBI Day 1 and Day 4 metabolites, respectively.

FIG. 7A and FIG. 7B show NMR data of prognosis of mortality and vegetative state for GOS-E 1&2 versus GOS-E 3-8 based on sTBI Day 1 and Day 4 metabolites, respectively.

FIG. 8A and FIG. 8B provide predictor screening analysis, showing the importance of clinical variables in the prediction models for the prognosis of GOSE outcome at 3 months and 12 months using DI/LC-MS/MS data. These figures present the ranking of metabolites and clinical variables in each prediction model.

### DETAILED DESCRIPTION

Generally, the present disclosure provides a method of determining a likelihood of a favourable or unfavourable outcome in a subject having traumatic brain injury (TBI) or suspected of having TBI, specifically a severe traumatic brain injury (sTBI).

The term “subject” or “patient” or “individual”, as used herein, refers to a eukaryote. A biological sample is typically obtained from a eukaryotic organism including, but not limited to, mammals. Mammalian subjects include, but are not limited to, primates such as a human; non-human primates including chimpanzees and the like; livestock, including but not limited to, cows sheep, pigs, and the like; companion animals, including but not limited to, dogs, cats, horses, rabbits, rodents including mice and rats, and the like.

In a specific example, the subject is a human.

The term “sample” or “biological sample” as used herein, encompasses a variety of cells, cell-containing bodily fluids, bodily fluids, and/or secretions as well as tissues including, but not limited to a cell(s), tissue, whole blood, blood-derived cells, plasma, serum, sputum, mucous, bodily discharge, and combinations thereof, and the like. Biological samples may include, but are not limited to, tissue and/or fluid isolated from a subject. Biological samples may also include sections of tissues such as biopsy and autopsy samples, formalin-fixed paraffin-embedded (FFPE) samples, frozen sections taken for histologic purposes, blood and blood fractions or products (e.g., serum, plasma, platelets, red blood cells, white blood cells and the like), sputum, stool, tears, mucus, hair, and skin. Biological samples also include explants and primary and/or transformed cell cultures derived from animal or patient tissues.

In certain examples, biological samples may also be blood, a blood fraction, urine, effusions, ascitic fluid, saliva, cerebrospinal fluid, cervical secretions, vaginal secretions, endometrial secretions, gastrointestinal secretions, bronchial secretions, sputum, cell line, tissue sample, or secretions from the breast.

In a specific example, a biological sample is a blood samples, or a blood fraction.

In a specific example, the biological sample is a serum sample.

A sample may be obtained from a subject.

As used herein, “obtaining a sample” or “obtaining a biological sample” refers to methods as will be well known to the skilled worker. A biological sample may be obtained directly or indirectly from the subject. The term “obtaining” a biological sample may comprise receiving a biological sample from an agent acting on behalf of the subject. For example, receiving a biological sample from a doctor, nurse, hospital, medical center, etc., either directly or indirectly, e.g. via a courier or postal service. In some cases the biological sample is obtained from archival repositories. In one example, the methods of the invention are carried out in vitro or ex vivo.

For example, a blood sample, such as a peripheral blood sample, may be collected using venipuncture.

A biological sample can be collected on more than one occasion.

The term “determining the likelihood” and “prediction” as used herein, refers to providing a measure of relative risk for developing an outcome, such as a favourable or unfavourable outcome from TBI in a subject.

The term “providing a prognosis”, as used herein, refers to providing a prediction of the probable course and outcome of TBI in a subject.

The term “diagnosis”, as used herein, refers to detecting a favourable or unfavourable outcome in a subject having TBI or at risk of TBI. It will be appreciated that typically any method of diagnosis includes false positives and false negatives. Accordingly, it is typical that a method of diagnosis does not necessarily provide 100% accuracy.

The term “traumatic brain injury” (TBI, and sTBI), as used herein, refers to a brain injury resulting from direct or indirect shock load or loads applied to the brain. The direct or indirect shock load or lads may cause the brain to move rapidly and unnaturally within a patient's skull. TBI includes, but not be limited to, brain injuries caused by: (a) objects penetrating the skull, such as, bullets, arrows, and other physical objects which pass through the skull and enter the brain, (b) impact loads applied to the head or other portions of the patient's body, (c) surgically induced trauma, (d) explosions, such as might exist in warfare, through impacting of grenades, bombs, and other explosives, which cause substantial tremors in the earth in relatively-close proximity to where an individual is standing, as well as similar tremors created by nonexplosive means, such as vehicular accidents, collapse of buildings and earthquakes, for example.

A traumatic brain injury may be categorized as severe traumatic brain injury (herein “sTBI”), the severity of which is a relative term based on GOSE score or other clinical parameters.

The term “normal patient(s)”, or “control patient(s)”, as used herein, refers to a patient without TBI, preferably matched for age and sex.

TBI outcome in a subject may be assessed using the Glasgow Coma Scale (GCS), and/or the Glasgow Outcome Scale Extended (GOSE).

In specific examples described and used herein, the primary outcome was Glasgow Outcome Scale Extended (GOSE) measured at 3 and 12 months post injury. GOSE 1-4 and GOSE 5-8 were considered as unfavorable and favorable outcomes, respectively. Another primary outcome was mortality at 3 months.

In some examples, the methods described herein may involve measuring a sample from a subject, such as a serum sample.

In some examples, the methods herein may involve determining if a patient having a TBI or suspected of having a TBI will have a favourable or unfavourable outcome.

In some example, a subject is assessed at about one (1) day and four (4) days following a TBI, or suspect TBI.

In some examples, a serum metabolite signature (which may also be referred to as a biosignature) may be used for the prognosis of GOSE outcome at 3 and 12 months and the mortality outcome at 3 months.

A metabolite signature (i.e., a population of cellular metabolites) differentially produced by TBI subject samples, such as serum, may provide a reliable diagnostic marker for determining a likelihood of a favourable outcome or unfavourable outcome.

The term “metabolite”, “cellular metabolite” or the plural form, “cellular metabolites,” as used herein refers to any molecule or mass feature in the range of about 10 Daltons to about 1500 Daltons secreted by a cell and present in a tissue sample or biological fluid. A cellular metabolite may include but is not limited to the following types of molecules: acids, bases, lipids, sugars, glycosides, amines, organic acids, lipids, amino acids, oximes, esters, dipeptides, tripeptides, fatty acids, cholesterols, oxysterols, glycerols, steroids, and/or hormones.

In one example, the metabolite is lysophosphatidylcholines (lysoPCs) and fatty acids such as propionic acid, stearic acid, oleic acid, linoleic acid, myristic acid as well as branched-chain and aromatic amino acids.

The phrases “identifying one or a plurality of cellular metabolites . . . differentially produced” and “differentially produces” as used herein include but are not limited to comparisons of cells, or tissues, or fluids, from a subject with TBI with cells or tissues from non-TBI subject.

Detection or measurement of variations in metabolite populations or mass features between TBI and non-TBI control samples are included in this definition.

In some examples, alterations in production of various metabolites are measured by determining a profile of changes in metabolite molecules in TBI versus control samples.

The term “physical separation method” as used herein refers to any method known to those with skill in the art sufficient to detect a profile of changes and differences in metabolites produced in the tissue or fluid (e.g., serum, lateral cerebellum, and post vermis brain, cerebrospinal fluid, blood, or plasma) of TBI subject.

In some examples, physical separation methods permit detection of cellular metabolites including but not limited to sugars, organic acids, amino acids, fatty acids, hormones, vitamins, and peptides, as well as ionic fragments thereof and other cellular metabolites (for example, having a molecular weight less than 3000 Daltons, more particularly between 10 and 1500 Daltons, and even more particularly between 100 and 1000 Daltons).

In some examples, proton nuclear magnetic resonance (1H-NMR) spectroscopy and/or tandem MS (MS/MS), such as targeted direct injection tandem mass spectrometry (DI-MS/MS) or DI/LC-MS/MS, were applied to identify and quantify metabolites in the serum samples.

Metabolites can be identified using their exact molecular mass, as well as mass spectrometry fragmentation patterns of the metabolites.

It will be understood that cellular metabolites as set forth herein can be detected using alternative spectrometry methods or other methods known in the art for analyzing these types of cellular compounds in this size range.

The term “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”).

The term “detect” refers to identifying the presence, absence or amount of the object to be detected.

The term “diagnosis” refers to determination of a pathologic state.

Method of the invention are conveniently practiced by providing the compounds and/or compositions used in such method in the form of a kit. Such kit preferably contains the composition. Such a kit preferably contains instructions for the use thereof.

### EXAMPLES

To gain a better understanding of the invention described herein, the following examples are set forth. It should be understood that these examples are for illustrative purposes only. Therefore, they should not limit the scope of this invention in anyway.

### Example 1

Serum-Based Metabolomics Improve Prognosis of Outcome in sTBI

### SUMMARY

This example was set up to address the question of whether serum-based metabolomics can improve the prognosis of outcome among adult patients with a sever traumatic brain injury (sTBI). It was found that in a prospective cohort study that included 59 adult patients with sTBI, serum metabolomics profiling on days 1 and 4 post-injury was associated with the prognosis of GOSE outcome in a highly predictive (Q2>0.5) and accurate (AUC>0.99) manner as well as being highly predictive of mortality. These findings indicate that metabolomics profiling on serum can be used for the prognosis of GOSE outcome in adult patients with sTBI at 3 and 12 months post injury and can help predict mortality at 3 months.

Importance. The prediction of outcomes and disease stratification are key problems for the management of sTBI. Currently clinical assessment and neuroimaging are the most reliable techniques for the prognosis of TBI, however, they are insufficiently sensitive and specific to adequately prognosticate outcome in sTBI.

Objectives. This Example is designed to determine whether the alteration of metabolites and metabolomics pattern in serum samples of sTBI in adult cohorts are associated with the prognosis of GOSE outcome.

Design. This study was carried out with the patients who met the clinical criteria for TBI, who were enrolled in the Canada TBI (CanTBI) platform.

Setting. This study was performed as a multicenter cohort study.

Participants. All enrolled patients were admitted to critical care units, and/or emergency departments and/or assessed in concussion clinics at 3 participating centers. In the adult arm, subjects were included if they had severe TBI (GCS≤8 with CT evidence of head injury) and were ≥18 years of age. Exclusion criteria consisted of any neurodevelopmental disorder pre-injury and/or an ongoing neurologic deficit from a previous head injury.

Main Outcome(s) and Measure(s). The primary outcome was Glasgow Outcome Scale Extended (GOSE) measured at 3 and 12 months post injury. GOSE 1-4 and GOSE 5-8 were considered as unfavorable and favorable outcomes, respectively. Another primary outcome was mortality at 3 months.

Results. Fifty-nine patients with sTBI were recruited and outcomes were measured at 3 and 12 months. Serum metabolic profiles were measured (including lipids) on days 1 and 4 post-injury and were found to be highly predictive (Q2>0.4) and highly accurate (AUC>0.99) to predict GOSE outcome at 3 and 12 months post-injury and mortality at 3 months. The metabolic profiles on day 4 were more predictive (Q2>0.55) than those measured on day 1 post-injury. Increased lysophosphatidylcholines, acylcarnitines, energy-related metabolites (glucose, lactate), aromatic amino acids and glutamate were associated with poor outcome and mortality.

Conclusions and Relevance. It was demonstrated that metabolomic profiles are strongly associated with prognosis of GOSE outcome at 3 and 12 months and mortality following sTBI in adults. The current findings strongly suggest that serum metabolomics can be more helpful than clinical data in determining prognosis in adults with sTBI in the early days post-injury. These findings clearly indicate utility in sTBI clinical management.

### Introduction

In this Example, it was hypothesized that serum metabolites (or “metabolomic biosignatures”) would be associated with favorable and unfavorable outcomes at 3 and 12 months and be associated with mortality in adults with sTBI. Our objectives were to measure metabolites in serum sampled at 1 and 4 days following severe TBI and determine if these metabolomic biomarkers significantly improve prognostic models using demographics, clinical factors, and CT findings to predict long-term outcomes. The study design of this Example tests whether the serum metabolites observed at day 1 and day 4 post-TBI allows prediction of outcome at 3 and/or 12 months post sTBI.

TBI Classification. Classification of TBI is based on severity, mechanisms or structural damage and pathophysiology. The following classifications are in place: Severity is generally indicated using Glasgow Coma Scale or “GCS” (ranging from 3-15): Mild (GCS 13-15); Moderate (GCS 9-13); Severe (GCS 3-8). Mechanism classification may be: primary or secondary injury. Structural classification may be: focal, diffuse or multifocal.

The importance of TBI in society is evidenced by the consequences of TBI, which are sizeable in both patients individual lives and economic terms. TBI is a leading cause of death and disability in people younger than 35 years of age and is increasing in the elderly. The prevalence: of TBI may be about 50 million cases worldwide. sTBI survivors usually exhibit lifelong disabilities involving both motor and cognitive domains. Annual costs of $76.5 billion in direct medical services and loss of productivity (indirect costs) have been estimated. Management of TBI is challenging. Mild TBI can be difficult to diagnose, while for severe TBI, it is often difficult to predict outcome, so as to guide not only clinical decisions but also personal decisions for the individual and family involved.

### Materials and Methods

Patients' Characteristics and Primary Clinical Information. In this prospective and multicenter cohort study, the patients who met the clinical criteria for TBI were enrolled in the CanTBI platform after informed consent was obtained from the patient or legal surrogate. Serum samples were collected and handled according to the CanTBI SOPs into 4 Canadian tissue banks. Samples were obtained at different days post-injury, while samples collected on days 1 and 4 post-injury from patients with sTBI were used in the current study. Extended Glasgow Outcome Scale (GOSE) was obtained using structured telephone interviews for the individual (or surrogate) at 3 and 12 months post-injury¹². A dichotomized GOSE approach 13.14 was used to predict favorably (GOSE 5-8) and unfavorable (GOSE 1-4) outcomes.

The CanTBI Platform is a National biobank and database for patients with traumatic brain injury (TBI) in Canada. This platform is designed to collect data and samples from TBI patients across Canada. As of 2021, data and samples from about 450 patients have been entered. Clinical Data, imaging and biosamples are collected for analysis. This is a source of patient samples and information as utilized herein.

Outcome measures commonly used in TBI assessment include: (1) Glasgow Outcome Scale Extended (GOS-E), which may be referenced herein interchangeably as “GOSE”, “GOS-E”, or GOSe; (2) Quality of Life After Brain Injury (QOLIBRI™); and (3) Pediatrics Quality of Life After Brain Injury (PedsQL™).

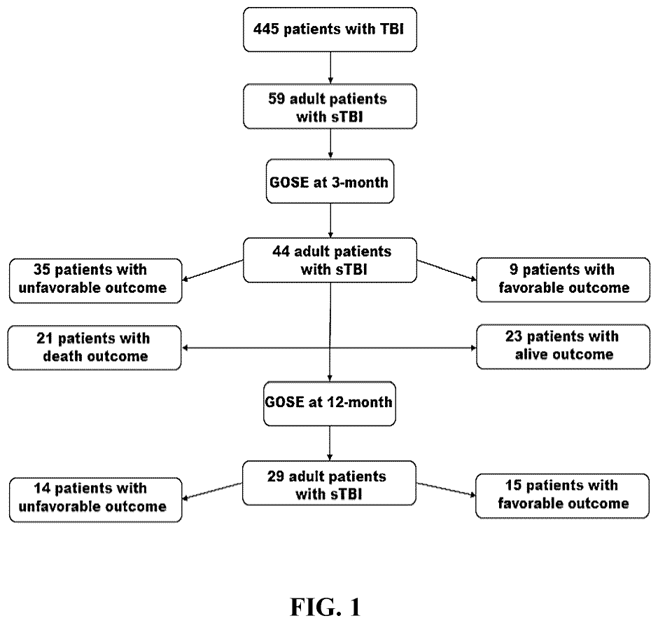

FIG. 1 is a diagram of the patient flow chart used for patient selection at baseline, and patients with measured GOSE outcome at 3 and 12 months.

Metabolomics Profiling, Quantification of Metabolites. Proton nuclear magnetic resonance (¹H-NMR) spectroscopy and tandem mass spectrometry (MS/MS) were applied to identify and quantify metabolites in the serum samples at days 1 and 4 post sTBI. These two techniques were used to quantify a broad list of metabolites with few overlapping metabolites. A comprehensive targeted analysis of 130 and 58 metabolites was carried out using MS/MS and ¹H-NMR, respectively, in the serum-based metabolic profiles of sTBI patients at days 1 and 4 post-injury.

Data and Statistical Analysis. For the prognosis of sTBI outcome, prediction models were developed using multivariate statistical analysis (MVA) and machine learning to separate sTBI patients with unfavorable outcomes from sTBI patients with favorable outcomes based on the serum metabolite profiles on days 1 and 4 post injury. In the MVA, principal component analysis (PCA) was carried out to examine the variability and trends of metabolic profiles among samples and partial least squares discriminant analysis (PLS-DA), a type of machine learning method was used to build prediction models 15. The prediction models were created using the most differentiating metabolites with a variable important of the projection (VIP) value>1.0. SIMCA-P v15.0.2 (Sartorius Stedim Biotech, Umea, Sweden) was used for the PLS-DA analysis. It was further analyzed whether clinical predictors or combining clinical predictors with the metabolomics data yielded a superior model to predict GOSE outcome. Statically inspired modification of partial least square (SIMPLS), an algorithm of the PLS method suitable for both nominal or continuous variables, was performed to develop prediction models using only clinical predictors or combined clinical with metabolites variables for the GOSE prognosis at 3 months, 12 months and for mortality. Developed prognostication models were characterized by the metrics R2 (goodness of model fit), Q2 (goodness of prediction), cross-validation p-value and permutation testing (200 times). Artificial Neural Network analysis (ANN) was performed to predict one response variable (unfavorable and/or favorable separately) using a flexible function of the input variables. JMP Pro 14.3.0 (SAS Institute Inc. USA) was used for SIMPLS and ANN analysis. MetaboAnalyst 4.0 (freeware available at www.metaboanalyst.ca) was used for multivariate and univariate analysis. AUC, sensitivity, and specificity were obtained using a multivariate approach included in each software package.

The data analysis of metabolomics, clinical data and combination of both for the predicting 3 and 12-month GOSE outcome was assessed using: Partial Least Squares-Discriminate Analysis (PLS-DA, also known as projection to latent structures)-based metabolomics prediction models obtained using SIMCA-P software were compared to Straightforward Implementation of a statistically inspired Modification to PLS (SIMPLS)-based metabolomics prediction models obtained using JMP software. SIMPLS-DA can be better for integer-related data whereas PLS-DA is better for continuous variables.

To choose the most differentiating metabolites, a Variable Importance of Projection (VIP)>1.0 approach was used for the PLS-DA and SIMPLS data. Q2 (goodness of model prediction) and R2 (goodness of model fit) are presented in cumulative form, consistent between the two PLS-DA and SIMPLS methods. All PLS-DA and SIMPLS prediction models use two components. Two components approach was used for clinical-based prediction models.

Two analytical platforms were assessed: Nuclear Magnetic Resonance Spectroscopy (NMR) and Direct Infusion Tandem Mass Spectroscopy (DI-MS/MS). To compare and contrast these two analytical platforms, data was compared. In general, NMR is known for reproducibility, and quantitative strengths. In general DI-MS/MS is known for the strengths of targeted quantitative analysis and the inclusion of lipid quantification in the analysis. Different metabolites were assessed, with 58 metabolites being assessed in NMR, and 130 metabolites (including 70 lipids) being assessed in DI-MS/MS. NMR metabolite analysis includes amino acids and sugars. DI-MS/MS analysis includes phosphatidylcholines, lysophosphatidylcholines, acylcarnitines, amino acids and amino acid derivatives.

### Results

sTBI Patient Characteristics. Out of the 445 adult and pediatric patients with TBI enrolled in the CanTBI platform, 59 (13.2%) patients with sTBI were diagnosed and enrolled in the current metabolomics study. The sTBI cohort included 48 males and 11 females with a mean age of 50 y (±SD, ±20.6). FIG. 1 shows the patient flow chart and patients selection with measured GOSE at 3 and 12 months post-injury. Tables 1 and 2 summarize the distribution and description of the patients' demographics, clinical information, GCS, GOSE and CT findings of the cohort with sTBI and patients with unfavorable outcome (GOSE 1-4) and patients with favorable outcome (GOSE 5-8) at 3 and 12 month, respectively. Of note, the table shows that the age and injury severity score (ISS) have a significantly positive correlation with the prognosis of the unfavorable outcome at 3 months. There was a significant difference in age and ISS between patients who died with sTBI (GOSE 1) (n=21) and those who survived STBI (GOSE≥3) (n=23) at 3 months (Table S1), with older age and higher injury severity score associated with unfavorable outcome. Predictive partition analysis determined the cut-off value for ISS≥75 and age≥49 for the separation of non-survival vs survival at 3 months. The data also suggested a cut-off value for the Marshall score=4 and GCS=6 between non-survivors and survivors, although these two variables were not statistically significantly different between the two cohorts.

Identified, Quantified Metabolites. 130 and 58 metabolites from different metabolite classes were identified and quantified using targeted DI-MS/MS and untargeted 1H-NMR, respectively. Most of the common metabolites (24 of 30) had a similar trend of change, showing the accuracy of both techniques.

Metabolomics for the Prognosis of 3 and 12 Months Outcomes of sTBI.

Prediction models showed that a serum metabolic biosignature can be used for the prognosis of GOSE outcome at 3 and 12 months and the mortality outcome at 3 months.

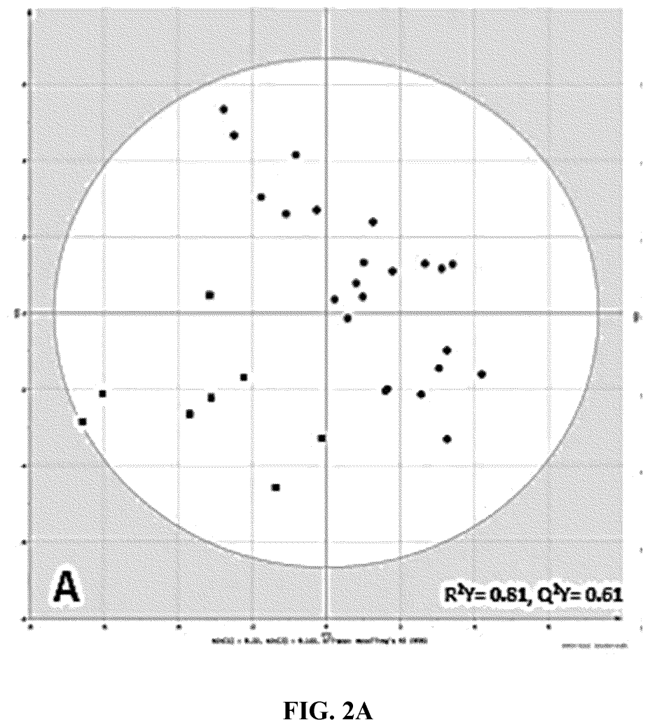

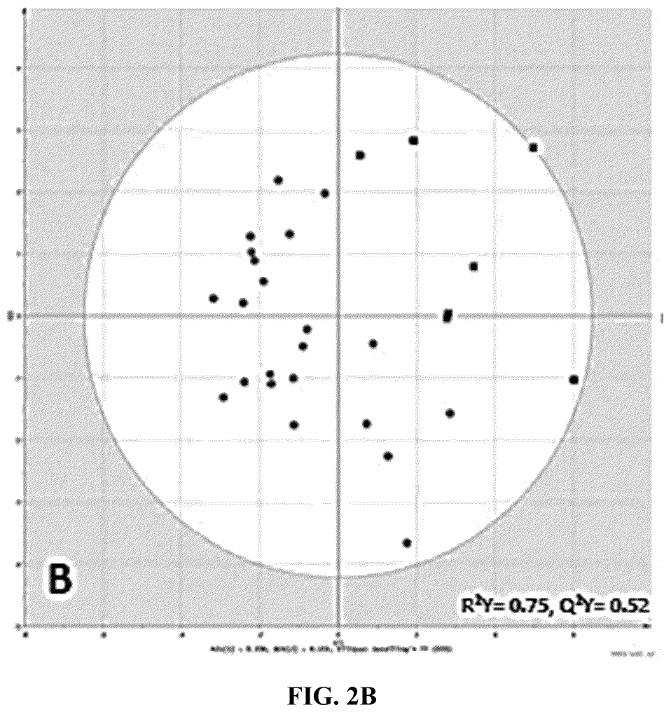

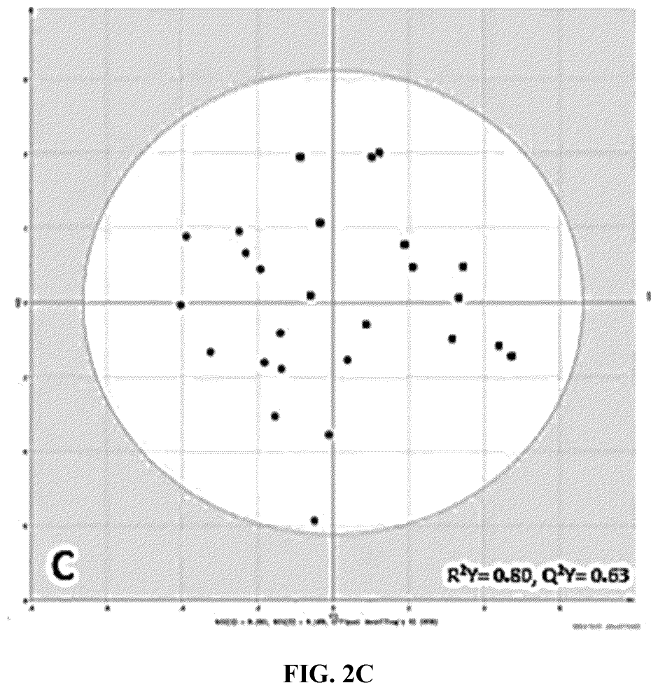

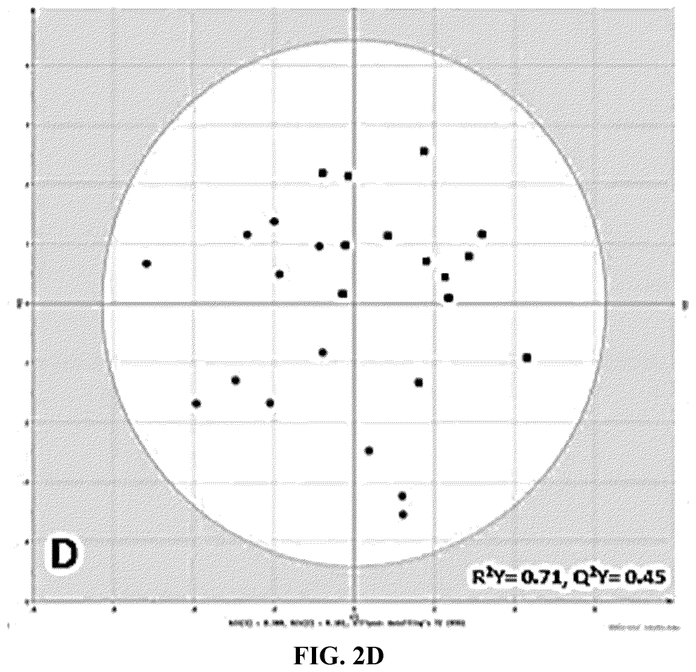

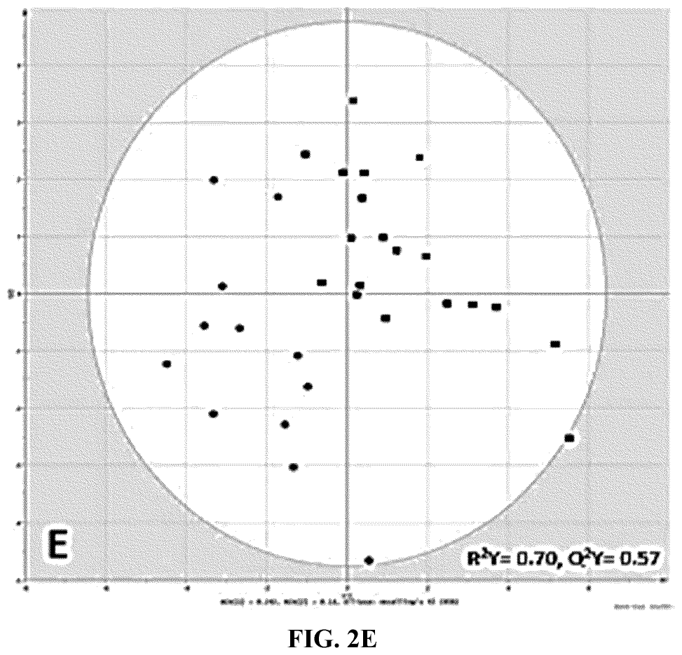

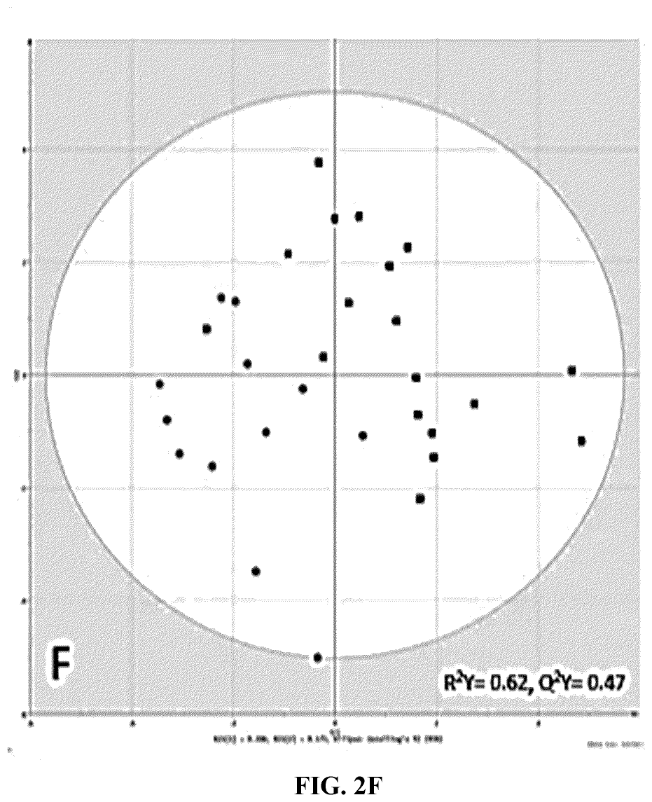

Using PCA, a high level of variability (R2X>0.5) was obtained in the metabolic biosignature between cohorts with different outcomes, implying a considerable impact of head injury on serum metabolic profiles on days 1 and 4 post-injury (Fig S3-S5). The observed metabolic variability was phenotypically characterized to visualize more clearly the grouping between the unfavorable vs. favorable GOSE and non-survivor vs. survivor outcomes on day 4 compared to day 1 post-injury for both DI-MS/MS and for 1H-NMR. The prognosis of sTBI outcomes was remarkably strong to distinguish the patients with the unfavorable outcome from patients with favorable outcome and non-survivor vs survivor outcomes based on metabolic biosignatures on day 4 (metabolites included lipid compounds) obtained by DI-MS/MS. The prediction models proved to be highly predictive (Q2>0.5) and highly significant (p-value<0.0001) (see Table 3 and FIGS. 2A, 2C, 2E). Importantly, prediction models revealed that metabolic biosignatures on day 4 post-injury were significant predictors for GOSE outcomes using the two metabolomics analytical platforms (FIGS. 2B, 2D, 2F). All prediction models were highly sensitive, specific (>99%) and highly predictive (AUC>0.99) (Table 3). The validity of the prediction models were verified using permutation analysis (200 times permuted, data not shown) strongly confirming that the models are valid and have not been overfit. Artificial neural network analysis (ANN) revealed that the prognosis of the unfavorable GOSE outcome was more predictable at 3 months and favorable GOSE outcome showed higher predictability at 12 months, (AUC>0.90) respectively. This is illustrated in DI-MS/MS data of day 4.

Characterization of Metabolite Biosignature for the Prognosis of GOSE Outcome. Further investigation showed that a highly predictive (Q2>0.5) and a high AUC (>0.99) was obtained for a list of metabolites (n=22-56) for the prognosis of sTBI outcome. For the model, if one considers using a decrease in the number of DI-MS/MS metabolites it was associated with less predictability (Q2>0.4) and AUC (>0.90) but there is a still an acceptable model for prediction of outcome. A further decrease in the number of metabolite numbers have lower sensitivity, specificity (<80%), and AUC (0.80). Overall, the metabolite biosignature for patients with unfavorable outcomes were characterized by increased lysophosphatidylcholines (lysoPCs) and fatty acids such as propionic acid, stearic acid, oleic acid, linoleic acid, myristic acid as well as branched-chain and aromatic amino acids.

Clinical Variables for the Prognosis of GOSE Outcome at 3 Months and 12 Months and Mortality at 3 Months. It was investigated whether clinical variables could predict the outcome of sTBI at 3 and 12 months post sTBI. The variables included gender, age, GCS, ISS (injury severity score), intubation, hypoxemia, hypotension, loss of consciousness and Marshall score for the prediction of GOSE outcome at 3 and 12 months, and for mortality at 3 months. Individual CT findings were not used in the prediction models as the Marshall score showed similar predictability to CT findings for the prognosis of outcome in the adult cohort (unpublished data from CanTBI investigators). Depending on the number of patients evaluated for 3 and 12 month outcomes, the SIMPLS method revealed age, ISS, Marshall score and hypoxemia were the most differentiating clinical variables (VIP>1.0) for predicting 3 month GOSE outcome (unfavorable vs favorable). Also, age, GCS, hypoxemia, and loss consciousness were the most differentiating variables (VIP>1.0) for predicting 12 month unfavorable vs favorable GOSE outcome. Despite identifying these differentiating clinical variables, the clinical variables had low prediction capacity (Q2<0.16) and less sensitivity and specificity (66%-86%) to predict the outcome at 3 and 12 months compared to metabolomics data (Table S9). SIMPLS analysis of the clinical data revealed that the age and severity of illness score (ISS) are useful predictors (Q2=0.37, AUC=0.86) for the prognosis of mortality. However, these clinical variables lack significant sensitivity and specificity (66%-83%) compared to metabolomics data.

The Combination of Metabolomics and Clinical Variable for the Predicting GOSE Outcome at 3 Months and 12 Months Post-Injury. Using SIMPLS analysis, it was demonstrated that the clinical features cannot significantly improve the performance of metabolomics-based prediction models for the prognosis of GOSE outcome at 3 months and mortality, however, clinical features were found to minimally improve the model for GOSE prognosis at 12 months. For the sTBI cohort, predictor screening analysis demonstrated that age could be considered as the most important clinical predictor of outcome, with a high level of contribution to almost all outcome prediction models, particularly for mortality, in association with other important metabolites followed by Marshall score (3 months outcome) and GCS (12 months outcome). Although SIMPLS and PLS-DA use different algorithms for determining prediction models, the two approaches showed overall similar predictabilities when metabolites were used for the prognosis sTBI outcomes, with only slight differences. Importantly, permutation tests (data not shown) verified the predictabilities of metabolite-based prediction models.

Table 1 shows the clinical data of patient characteristics and categorization for n=59 patients having severe traumatic brain injury (sTBI). The patients enrolled were 59 adult sTBI patients from across Canada (GOSE 3 months n=44; GOSE 12 months n=29). Serum was collected on day 1 (to reflect primary injury, n=59) and on day 4 post-sTBI (as a reflection of possible secondary injury, n=44). Age-matched and sex-matched orthopedic injury (OI) controls without head injury were also enrolled, with samples from University of British Columbia (Vancouver, Canada). Day 1 serum samples were collected from OI controls. Patients' characteristics, clinical information, GCS at admission, GOSE outcome distribution, CT findings, and Marshall score are shown. In Table 1, “*” indicates the number of patients with the clinical information and the percentage of total patients, others included without clinical information, and missing information; “**” indicates the number of patients (percentage of total) were included in the same GCS categorized level; “‡” indicates the number of patients with GOSE data at the same time; and “‡‡” indicates the number of patients that had the same CT findings; the rest may include patients without CT findings or findings missing in the study.

TABLE 1
Patient Characteristics and Categorization

| | | n = 59 severe |
|---|---|---|
| Patients Characteristics | Subcategory/unit | TBI |
| Sex | Male/Female | 48/11 |
| Age | Mean (±SD) | 50 ± 20.6 |
| Weight | Mean (±SD) | 82 ± 19.0 |
| Admission type | n (%) | |
| ER | | 19 (32.3) |
| ICU | | 40 (67.7) |
| Severity (ISS) | Mean (±SD) | 43.3 ± 19 |
| Intubated | Yes (%)* | 40 (67.7) |
| Hypoxia | Yes (%)* | 8 (13.5) |
| Hypotension | Yes (%)* | 9 (15.2) |
| Paralytic agent | Yes (%)* | 30 (50.8) |
| Loss of Consciousness | Yes (%)* | 40 (67.7) |
| GCS (total) | Mean (±SD) | 5.46 ± 2.27 |
| GCS-Motor | | 2.87 ± 2.07 |
| GCS-Eye | | 1.54 ± 1.02 |
| GCS-Verbal | | 0.98 ± 0.71 |
| GCS (categorized) | n (%)** | |
| GCS 3-4 | | 26 (44) |
| GCS 5-6 | | 6 (6.7) |
| GCS 7-8 | | 26 (44) |
| GOSE | | |
| 3-month | | 44 (74.5) |
| Poor | | 35 (59.3) |
| Good | | 9 (15.2) |
| 6-month | n (%)^{†} | 22 (37.2) |
| Poor | | 9 (15.2) |
| Good | | 13 (22) |
| 12-month | | 29 (49.7) |
| Poor | | 14 (23.7) |
| Good | | 15 (25.4) |
| GOSE 1 & 2 (3 month) | n (%) | 21 (35.5) |
| CT Findings | (Yes/No) ^{‡‡} | |
| Diffuse Axonal Injury | | 35/7 |
| Mild Shift | | 14/26 |
| Skull Fracture | | 28/14 |
| Cerebral Edema | | 10/32 |
| Contusion | | 18/24 |
| Intracranial Hemorrhage | | 26/16 |
| Epidural Hemorrhage | | 5/37 |
| Subdural Hemorrhage | | 30/12 |
| Arachnoid Hemorrhage | | 32/10 |
| Marshall Score | n (%) | |
| I | | 1 (2.3) |
| II | | 23 (54.7) |
| III | | 6 (14.2) |
| IV | | 5 (11.9) |
| V | | 7 (16.6) |

Table 2 shows data obtained regarding prognosis of GOSE outcome at 3 and 12 months. Patient demographics and clinical characteristics for unfavorable (GOSE 1-4) and favorable (GOSE 5-8) outcome groups at 3 and 12 months are shown. In Table 2, “*” indicates the variables are based on the number of patients; “‡” indicates that data includes several variables that have not been shown in detail for each cohort. There was no significant difference for any type and location of injury between cohorts with favorable and unfavorable outcome at 3 and 12 months post injury.

TABLE 2
Patient Demographics and Prognosis of GOS-E Outcome at 3 and 12 Months

| Prognosis of GOSE | | | | | | |
|---|---|---|---|---|---|---|
| outcome | 3 Month | | | 12 Month | | |
| | Poor | Good | | Poor | Good | |
| Patients Characteristics | Outcome | Outcome | p | Outcome | Outcome | p |
| and clinical information | (n = 35) | (n = 9) | value | (n = 14) | (n = 15) | value |
| Sex (Male/Female) | 30/5 | 6/3 | 0.42 | 11/3 | 13/2 | 0.82 |
| Age (mean ± SD) | 55.4 ± 20.4 | 40.5 ± 21.0 | 0.03 | 52.0 ± 18.7 | 38 ± 19.8 | 0.06 |
| Weight (mean ± SD) | 88.5 ± 19.5 | 76.4 ± 21.1 | 0.08 | 81.7 ± 22.6 | 79.3 ± 16.1 | 0.75 |
| Injury Severity Score (ISS) | 56.4 ± 22.6 | 35.1 ± 12.6 | <0.01 | 35.5 ± 12.5 | 36.4 ± 12.5 | 0.81 |
| (mean ± SD) | | | | | | |
| Admission-type | 13 | 2 (22.2%) | 0.36 | 4 (28.5%) | 4 (26.6%) | 0.58 |
| ER | (37.1%) | 7 (77.7%) | | 10 (71.4%) | 11 (73.3%) | |
| ICU | 21 (60%) | | | | | |
| Hypoxia (Yes/No)* | 8/22 | 0/9 | 0.07 | 3/8 | 1/14 | 0.38 |
| Intubated (Yes/No)* | 21/13 | 7/2 | 0.61 | 11/3 | 10/5 | 0.77 |
| Hypotension (Yes/No)* | 5/25 | 1/7 | 0.98 | 2/10 | 2/13 | 0.64 |
| Paralytic-AGT (Yes/No)* | 16/17 | 6/1 | 0.40 | 6/7 | 9/4 | 0.32 |
| Loss Consciousness* | 25/4 | 5/2 | 0.30 | 13/0 | 8/2 | 0.48 |
| Location of Injury ^{t} | | | 0.70 | | | 0.52 |
| Type of Injury ^{t} | | | 0.24 | | | 0.21 |
| GCS (total) (mean ± SD) | 5.3 ± 2.17 | 5.3 ± 2.5 | 0.95 | 4.5 ± 1.9 | 5.8 ± 2.3 | 0.11 |
| GCS-Motor | 2.9 ± 1.9 | 2.4 ± 2.2 | 0.54 | 2.28 ± 2.0 | 2.7 ± 2.1 | 0.57 |
| GCS-Eye (mean ± | 1.5 ± 1.1 | 1.0 ± 0.0 | 0.14 | 1.4 ± 0.99 | 1.6 ± 1.3 | 0.68 |
| SD) | 1.0 ± 0.75 | 1.1 ± 0.78 | 0.94 | 0.71 ± 0.48 | 1.13 ± 0.74 | 0.08 |
| GCS-Verbal | | | | | | |
| GCS 3-4 | 15 | 5 (55.5%) | 0.80 | 9 (64.2%) | 6 (40%) | 0.62 |
| GCS 5-6 (mean ± | (42.5%) | 0 | | 1 (7.1%) | 1 (6.6%) | |
| SD) | 6 (14.1%) | 4 (44.4%) | | 4 (28.5%) | 8 (53.3%) | |
| GCS 7-8 | 14 (40%) | | | | | |
| CT Findings** | 5/20 | 0/7 | 0.57 | 2/7 | 2/11 | 0.62 |
| Diffuse Axonal Injury | 6/18 | 4/3 | 0.41 | 4/4 | 6/7 | 0.16 |
| Mid Shift | 209/6 | 3/4 | 0.30 | 7/2 | 9/4 | 0.52 |
| Skull Fracture | 6/20 | 0/7 | 0.24 | 3/6 | 2/11 | 0.49 |
| Cerebral Edema | 14/12 | 2/5 | 0.33 | 3/6 | 7/6 | 0.47 |
| Contusion | 18/8 | 3/4 | 0.53 | 5/4 | 8/5 | 0.63 |
| Intracranial Hemorrhage | 3/23 | 0/7 | 0.62 | 7/2 | 9/4 | 0.11 |
| Epidural Hemorrhage | 20/6 | 5/2 | 0.34 | 8/2 | 8/4 | 0.86 |
| Subdural Hemorrhage | 22/4 | 5/2 | 0.37 | 6/3 | 8/5 | 0.16 |
| Arachnoid Hemorrhage | | | | | | |
| Marshall Score | 0 | 0 | | 0 | 0 | |
| I | 17 | 3 | | 4 | 6 | |
| II | 4 | 1 | 0.19 | 1 | 2 | 0.37 |
| III | 3 | 0 | | 3 | 1 | |
| IV | 2 | 3 | | 1 | 4 | |
| V | | | | | | |

Table 3 shows DI-MS/MS Data of Day 1 samples, GOS-E Poor Outcome and Good outcome at 3 months.

TABLE 3
DI-MS/MS data, Day 1 Samples,
GOSE Poor Outcome and Good Outcome at 3 Months

| | | | | | | | | Variables | |
|---|---|---|---|---|---|---|---|---|---|
| | Method | Models | Q2 | R2Y | Sensitivity | Specificity | AUROC | (#) | Patients |
| DI- | PLS-DA | Metabolomics | 0.39 | 0.74 | 100 | 100 | 1 | 48ª | Poor |
| MS/MS | SIMPLS | Metabolomics | 0.45 | 0.61 | 98 | 99 | 1 | 48ª | outcome, |
| Day 1 | | Clinical | 0.17 | 0.26 | 86 | 66 | .85 | 4^{b} | n = 35 |
| GOSE | | Variables | | | | | | | Good |
| outcome | | Combination | 0.56 | 0.68 | 100 | 92 | 1 | 48ª + 4^{b} | outcome |
| at 3 | | metabolomics | | | | | | | n = 9 |
| months | | and clinical | | | | | | | |
| | | variables | | | | | | | |

^{a}Metabolites;
^{b}Clinical variables = Age, Severity, Marshall Score, Hypoxemia

Table 4 shows DI-MS/MS Data of Day 1 samples, GOS-E Poor Outcome and Good outcome at 12 months.

TABLE 4
DI-MS/MS data, Day 1 Samples,
GOSE Poor Outcome and Good Outcome at 12 Months

| | | | | | | | | Variables | |
|---|---|---|---|---|---|---|---|---|---|
| | Method | Models | Q2 | R2Y | Sensitivity | Specificity | AUROC | (#) | Patients |
| DI- | PLS-DA | Metabolomics | 0.55 | 0.83 | 100 | 100 | 1 | 43ª | Poor |
| MS/MS | SIMPLS | Metabolomics | 0.61 | 0.84 | 98 | 99 | 1 | 43ª | outcome, |
| Day 1 | | Clinical | 0.13 | 0.30 | 78 | 73 | 79 | 4^{b} | n = 14 |
| GOSE | | Variables | | | | | | | Good |
| outcome | | Combination | 0.64 | 0.84 | 100 | 100 | .99 | 43ª + 4^{b} | outcome |
| at 12 | | metabolomics | | | | | | | n = 15 |
| months | | and clinical | | | | | | | |
| | | variables | | | | | | | |

^{a}Metabolites;
^{b}clinical variables = Age, GCS, Hypoxemia, Loss of Consciousness

Table 5 shows the relative importance of metabolites as predictive biomarkers of sTBI outcome at 3 months.

TABLE 5
The Prediction of sTBI outcome at 3 Months

| | number of | | |
|---|---|---|---|
| | metabolites | Most important | |
| | for each set | metabolites | Name of Metabolites |
| The | 15 | Acylcarnitines | C3:1, LysoPC 17:0, C14:1OH, |
| minimum | | (ACs) and | LysoPC 18:0, C18:2, |
| set of | | Lysophosphatidylcholines | LysoPC16:0, C14, Lactate, |
| Biomarkers | | (LysoPCs) | Glutamate, Dimethylarginine, |
| | | | Citrulline, Ornithine, Citric acid, Uric |
| | | | acid, Kynurenine |
| The middle | 30 | Acylcarnitines, | C3:1, LysoPC 17:0, C14:1OH, |
| set of | | Lysophosphatidylcholines | LysoPC 18:0, C18:2, |
| biomarkers | | and excitatory | LysoPC16:0, C14, Lactate, |
| | | neurotransmitters | Glutamate, Dimethylarginine, |
| | | such Glutamate, | Citrulline, Ornithine, Citric acid, Uric |
| | | Tyrosine, | acid, Kynurenine, Aspartate, tyrosine, |
| | | Phenylalanine | tryptophan, histidine, C5MDC, |
| | | | Choline, Succinate, Isoleucine, |
| | | | C18:1OH, LysoPC 17:0, LysoPC18:1, |
| | | | Pyruvate, Methionine, C4OH, |
| | | | Phenylalanine |
| The | 50 | Acylcarnitines | C3:1, LysoPC 17:0, C14:1OH, |
| maximum | | (ACs), | LysoPC 18:0, C18:2, |
| set of | | Lysophosphatidylcholines | LysoPC16:0, C14, Lactate, |
| biomarkers | | (LysoPCs), excitatory | Glutamate, Dimethylarginine, |
| | | neurotransmitters | Citrulline, Ornithine, Citric acid, Uric |
| | | such Glutamate, | acid, Kynurenine, Aspartate, tyrosine, |
| | | Tyrosine, | tryptophan, histidine, C5MDC, |
| | | Phenylalanine, | Choline, Succinate, Isoleucine, |
| | | Asparagine, | C18:1OH, LysoPC 17:0, LysoPC18:1, |
| | | Phosphatidylcholines | Pyruvate, Methionine, C4OH, |
| | | (PCs), Lactate, | Phenylalanine, C14:1, Homocysteine, |
| | | Pyruvate, | C2, C6:1, Threonine, C3, |
| | | Citrulline, | PC40:1aa, PC40:2aa, Betaine, |
| | | Ornithine, Uric | Fumarate, C16:2, Alanine, C5, C9, |
| | | acid, Kynurenine | LysoPC20:3, PC36:Oaa |

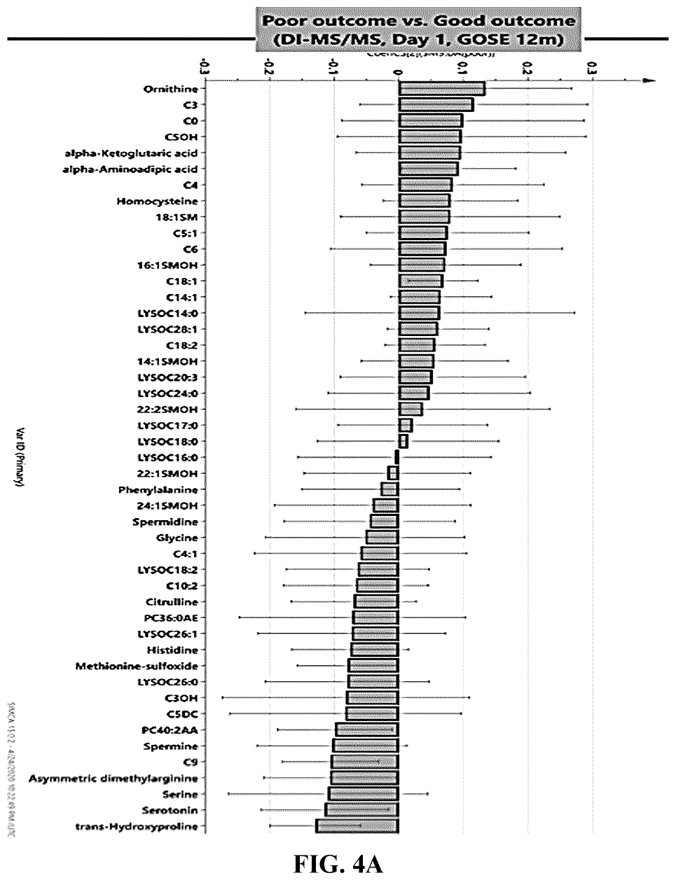

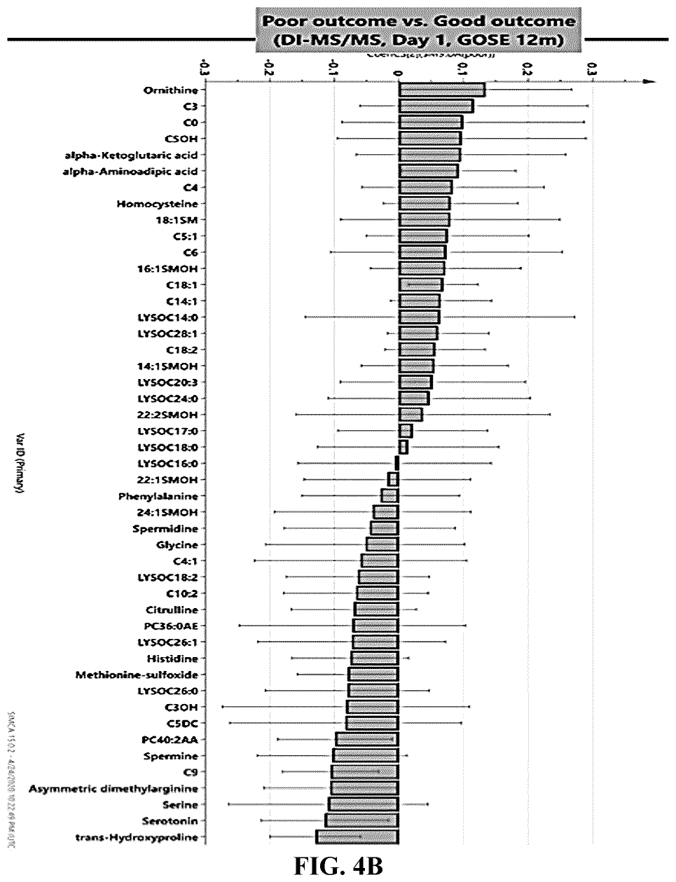

FIG. 4A and FIG. 4B show DI-MS/MS data of prognosis of GOS-E at 12 months for poor outcome versus good outcome based on TBI Day 1 and Day 4 metabolites, respectively.

As observed within the DI-MS/MS data presented in FIG. 4A and FIG. 4B, pertaining to prognosis of GOS-E at 12 months based on Day 1 serum, the primary increased metabolites include: ornithine, α-ketoglutaric acid, α-aminoadipic acid, homocysteine, and LysoPCs; and the primary decreased metabolites include: hydroxyproline, serotonin, dimethylarginine, α-aminoadipic acid, homocysteine, and LysoPCs.

For the DI-MS/MS data, pertaining to prognosis of GOS-E at 12 months based on Day 4 serum, the primary increased metabolites include: tryptophan, tyrosine, valine, kynurenine, alanine, and uric acid; and the primary decreased metabolites include: serotonin, spermine, and β-hydroxybutyric acid.

Table 6 shows DI-MS/MS Data of Day 4 samples, GOS-E Poor Outcome and Good outcome at 3 months.

TABLE 6
DI-MS/MS data, Day 4 Samples,
GOSE Poor Outcome and Good Outcome at 3 Months

| | | | | | | | | Variables | |
|---|---|---|---|---|---|---|---|---|---|
| | Method | Models | Q2 | R2Y | Sensitivity | Specificity | AUROC | (#) | Patients |
| DI- | PLS-DA | Metabolomics | 0.39 | 0.74 | 100 | 100 | 1 | 54ª | Poor |
| MS/MS | SIMPLS | Metabolomics | 0.45 | 0.75 | 98 | 97 | 1 | 54ª | outcome, |
| Day 4 | | Clinical | 0.26 | 0.40 | 75 | 82 | .82 | 3^{b} | n = 23 |
| GOSE | | Variables | | | | | | | Good |
| outcome | | Combination | 0.56 | 0.80 | 100 | 92 | .98 | 54ª + 3^{b} | outcome |
| at 3 | | metabolomics | | | | | | | n = 8 |
| months | | and clinical | | | | | | | |
| | | variables | | | | | | | |

^{a}Metabolites;
^{b}Clinical variables = Age, Marshall Score, Hypoxemia

Table 7 shows DI-MS/MS Data of Day 4 samples, GOS-E Poor Outcome and Good outcome at 12 months.

TABLE 7
DI-MS/MS data, Day 4 Samples,
GOSE Poor Outcome and Good Outcome at 12 Months

| | | | | | | | | Variables | |
|---|---|---|---|---|---|---|---|---|---|
| | Method | Models | Q2 | R2Y | Sensitivity | Specificity | AUROC | (#) | Patients |
| DI- | PLS-DA | Metabolomics | 0.63 | 0.83 | 100 | 100 | 1 | 31ª | Poor |
| MS/MS | SIMPLS | Metabolomics | 0.51 | 0.71 | 73 | 100 | 1 | 31ª | outcome, |
| Day 4 | | Clinical | 0.31 | 0.36 | 71 | 78 | .79 | 4^{b} | n = 13 |
| GOSE | | Variables | | | | | | | Good |
| outcome | | Combination | 0.54 | 0.75 | 100 | 100 | 1 | 31ª + 4^{b} | outcome |
| at 12 | | metabolomics | | | | | | | n =13 |
| months | | and clinical | | | | | | | |
| | | variables | | | | | | | |

^{a}Metabolites;
^{b}Clinical variables = Age, GCS, Gender, Loss of Consciousness

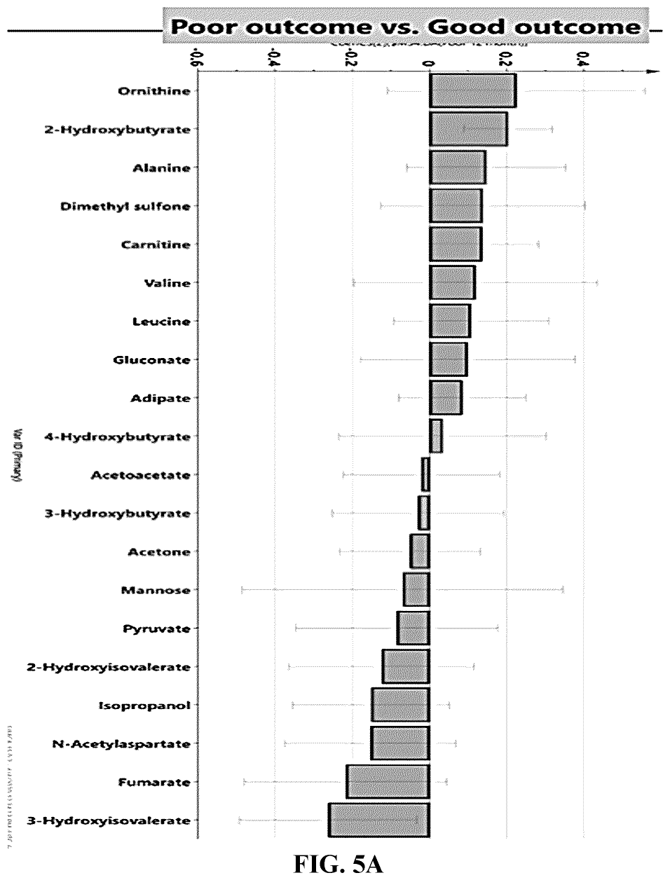

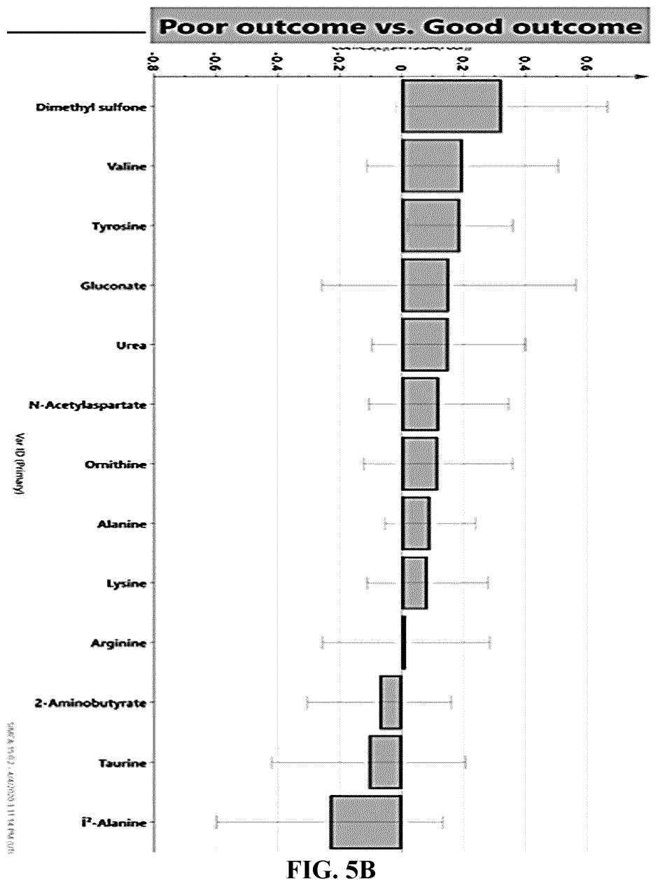

FIG. 5A and FIG. 5B show NMR data of prognosis of GOS-E at 12 months for poor outcome versus good outcome based on TBI Day 1 and Day 4 metabolites, respectively.

As observed within the NMR data presented in FIG. 5A and FIG. 4B, pertaining to prognosis of GOS-E at 12 months based on Day 1 serum, the primary increased metabolites include: ornithine, alanine, dimethyl sulfone, carnitine, valine, leucine, and adipate; and the primary decreased metabolites include: NAA, pyruvate, and mannose.

For the NMR data, pertaining to prognosis of GOS-E at 12 months based on Day 4 serum, the primary increased metabolites include: dimethyl sulfone, valine, tyrosine, gluconate, urea, NAA, ornithine, and alanine; and the primary decreased metabolites include: β-alanine, taurine, and arginine.

Table 8 shows the prediction of sTBI outcome at 12 months.

TABLE 8
The Prediction of sTBI outcome at 12 Months

| | Number of | | |
|---|---|---|---|
| | metabolites | Most important | |
| | for each set | metabolites | Name of Metabolites |
| The | 15 | Acylcarnitines (ACs) and | C3:1, Ornithine, CO, |
| minimum | | Glutamate, Spermine, | Homocysteine, |
| set of | | Ornithine, Lactate, | C4, C16, trans-hydroxyproline, |
| Biomarkers | | Gluconate, 3- | Spermine, Acetyl-ornithine serine, |
| | | hydroxisobutyrate | C6, C3OH, Tryptophan, C18, |
| | | | Betaine |
| The middle | 25 | Acylcarnitines, Glutamate, | C3:1, Ornithine, CO, |
| set of | | Spermine, Ornithine and | Homocysteine, |
| biomarkers | | Lysophosphatidylcholines, | C4, C16, trans-hydroxyproline, |
| | | Lactate, Gluconate, Valine | Spermine, Acetyl-ornithine serine, |
| | | | C6, C3OH, Tryptophan, C18, |
| | | | Betaine, LysoPC 28:1, C18:2, |
| | | | C18:1, C6, C5, Creatinine, |
| | | | Serotonin, C7DC, Spermine, |
| | | | Tyrosine, |
| The | 40 | Acylcarnitines (ACs), | C3:1, Ornithine, CO, |
| maximum | | Lysophosphatidylcholines | Homocysteine, |
| set of | | (LysoPCs), excitatory | C4, C16, trans-hydroxyproline, |
| biomarkers | | neurotransmitters such | Spermine, Acetyl-ornithine serine, |
| | | Glutamate, Tyrosine, | C6, C3OH, Tryptophan, C18, |
| | | Tryptophan, Serine, | Betaine, LysoPC 28:1, C18:2, |
| | | Lactate, Gluconate, | C18:1, C6, C5, Creatinine, |
| | | | Serotonin, C7DC, Spermine, |
| | | | Tyrosine, C12:1, LysoPC 14:0, |
| | | | Glutamate, C4:1 LysoPC 26:1, |
| | | | Spermidine, PC40:6ae, PC38:0aa, |
| | | | PC40:2aa, C16OH, C14, B, |
| | | | hydroxybutyric acid, alanine, |
| | | | LysoPC18:0, LysoPC28:1, |
| | | | LysoPC16:0 |

Table 9 shows NMR Data of Day 1 samples, GOS-E Poor Outcome and Good outcome at 3 months.

TABLE 9
NMR data, Day 1 Samples,
GOSE Poor Outcome and Good Outcome at 3 Months

| | | | | | | | | Variables | |
|---|---|---|---|---|---|---|---|---|---|
| | Method | Models | Q2 | R2Y | Sensitivity | Specificity | AUROC | (#) | Patients |
| NMR | PLS-DA | Metabolomics | 0.21 | 0.49 | 99 | 87 | 1 | 22ª | Poor |
| Day 1 | SIMPLS | Metabolomics | 0.35 | 0.55 | 91 | 89 | 1 | 22ª | outcome, |
| GOSE | | Clinical | 0.20 | 0.27 | 88 | 66 | 0.89 | 4^{b} | n = 35 |
| outcome | | Variables | | | | | | | Good |
| at 3 | | Combination | 0.48 | 0.54 | 100 | 70 | 0.95 | 22ª + 4^{b} | outcome |
| months | | metabolomics | | | | | | | n = 9 |
| | | and clinical | | | | | | | |
| | | variables | | | | | | | |

^{a}Metabolites;
^{b}Clinical variables = Age, Severity, Marshall Score, Hypoxemia

Table 10 shows NMR Data of Day 1 samples, GOS-E Poor Outcome and Good outcome at 12 months.

TABLE 10
NMR data, Day 1 Samples,
GOSE Poor Outcome and Good Outcome at 12 Months

| | | | | | | | | Variables | |
|---|---|---|---|---|---|---|---|---|---|
| | Method | Models | Q2 | R2Y | Sensitivity | Specificity | AUROC | (#) | Patients |
| NMR | PLS-DA | Metabolomics | 0.44 | 0.73 | 73 | 93 | 0.93 | 24ª | Poor |
| Day 1 | SIMPLS | Metabolomics | 0.56 | 0.76 | 81 | 89 | 0.93 | 24ª | outcome, |
| GOSE | | Clinical | 0.07 | 0.26 | 73 | 71 | 0.78 | 3^{b} | n = 14 |
| outcome | | Variables | | | | | | | Good |
| at 12 | | Combination | 0.51 | 0.74 | 91 | 100 | 0.98 | 24ª + 3^{b} | outcome |
| months | | metabolomics | | | | | | | n =15 |
| | | and clinical | | | | | | | |
| | | variables | | | | | | | |

^{a}Metabolites;
^{b}clinical variables = Age, Hypoxemia, Loss of Consciousness

Table 11 shows NMR Data of Day 4 samples, GOS-E Poor Outcome and Good outcome at 3 months.

TABLE 11
NMR data, Day 4 Samples,
GOSE Poor Outcome and Good Outcome at 3 Months

| | | | | | | | | Variables | |
|---|---|---|---|---|---|---|---|---|---|
| | Method | Models | Q2 | R2Y | Sensitivity | Specificity | AUROC | (#) | Patients |
| NMR | PLS-DA | Metabolomics | 0.52 | 0.75 | 100 | 90 | 0.99 | 26ª | Poor |
| Day 4 | SIMPLS | Metabolomics | 0.61 | 0.71 | 95 | 93 | 0.95 | 26ª | outcome, |
| GOSE | | Clinical | 0.25 | 0.36 | 85 | 50 | 0.83 | 3^{b} | n = 23 |
| outcome | | Variables | | | | | | | Good |
| at 3 | | Combination | 0.66 | 0.76 | 100 | 100 | 1 | 26ª + 3^{b} | outcome |
| months | | metabolomics | | | | | | | n = 8 |
| | | and clinical | | | | | | | |
| | | variables | | | | | | | |

aMetabolites;
bClinical variables = Age, Marshall Score, Hypoxemia

Table 12 shows NMR Data of Day 4 samples, GOS-E Poor Outcome and Good outcome at 12 months.

TABLE 12
NMR data, Day 4 Samples,
GOSE Poor Outcome and Good Outcome at 12 Months

| | | | | | | | | Variables | |
|---|---|---|---|---|---|---|---|---|---|
| | Method | Models | Q2 | R2Y | Sensitivity | Specificity | AUROC | (#) | Patients |
| NMR | PLS-DA | Metabolomics | 0.45 | 0.71 | 94 | 92 | 0.95 | 18ª | Poor |
| Day 4 | SIMPLS | Metabolomics | 0.51 | 0.71 | 92 | 91 | 0.92 | 18ª | outcome, |
| GOSE | | Clinical | 0.17 | 0.39 | 85 | 54 | 0.77 | 3^{b} | n = 13 |
| outcome | | Variables | | | | | | | Good |
| at 12 | | Combination | 0.51 | 0.71 | 82 | 65 | 0.81 | 18ª + 3^{b} | outcome |
| months | | metabolomics | | | | | | | n = 13 |
| | | and clinical | | | | | | | |
| | | variables | | | | | | | |

^{a}Metabolites;
^{b}Clinical variables = Age, GCS, Gender, Loss of Consciousness

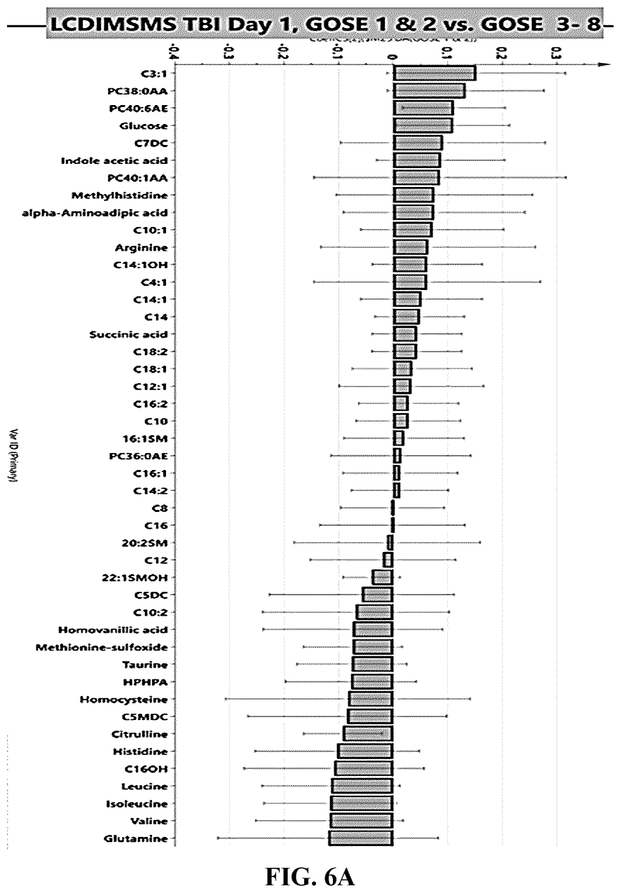

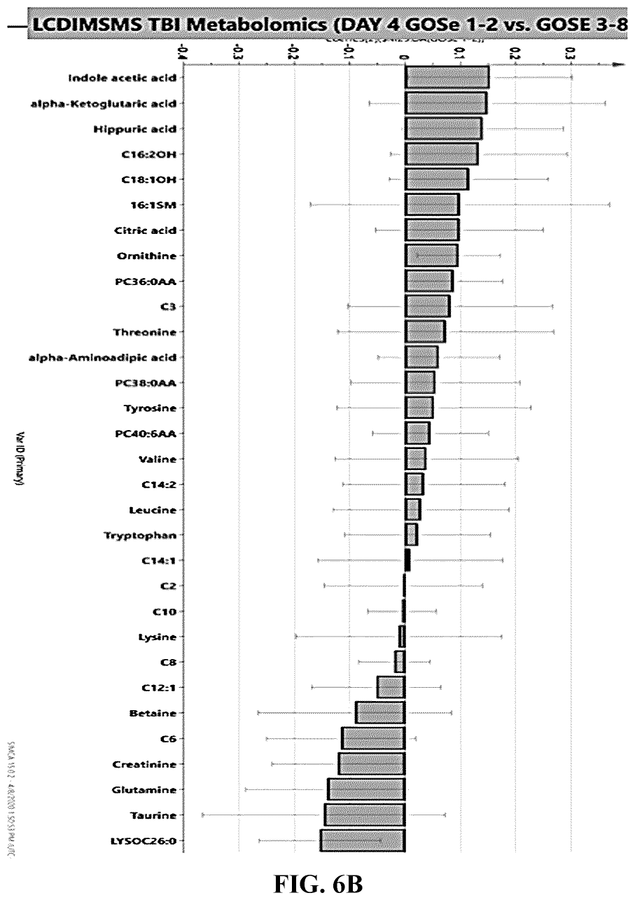

FIG. 6A and FIG. 6B show DI-MS/MS data of prognosis of mortality and vegetative state for GOS-E level 1-2 versus GOS-E level 3-8 based on TBI Day 1 and Day 4 metabolites, respectively.

As observed within the DI-MS/MS data pertaining to prognosis of mortality and vegetative state (died vs alive) based on Day 1 serum, the primary increased metabolites include: acylcarnitines, glucose, methyl histidine, α-aminoadipic acid and arginine; and the primary decreased metabolites include: glutamine, valine, isoleucine, histidine, citrulline, homocysteine, and homovanillic acid.

As observed within the DI-MS/MS data pertaining to prognosis of mortality and vegetative state (died vs alive) based on Day 4 serum, the primary increased metabolites include: indoleacetic acid, α-ketoglutaric acid, hippuric acid, acylcarnitines, citric acid, ornithine, threonine, valine, and tryptophan; and the primary decreased metabolites include: taurine, glutamine, creatinine, C6, and betaine.

Table 13 shows DI-MS/MS Data of Day 1 samples, relating to mortality outcome.

TABLE 13
DI-MS/MS data, Day 1 Samples, Mortality Outcome

| | | | | | | | | Variables | |
|---|---|---|---|---|---|---|---|---|---|
| | Method | Models | Q2 | R2Y | Sensitivity | Specificity | AUROC | (#) | Patients |
| DI- | PLS-DA | Metabolomics | 0.49 | 0.72 | 100 | 100 | 1 | 45ª | Died, |
| MS/MS | SIMPLS | Metabolomics | 0.71 | 0.82 | 94 | 96 | 1 | 45ª | n = 21 |
| Day 4 | | Clinical | 0.37 | 0.44 | 66 | 78 | 0.87 | 2^{b} | Alive, |
| mortality | | Variables | | | | | | | n = 23 |
| outcome | | Combination | 0.82 | 0.86 | 93 | 100 | 0.99 | 45ª + 2^{b} | |
| | | metabolomics | | | | | | | |
| | | and clinical | | | | | | | |
| | | variables | | | | | | | |

^{a}Metabolites;
^{b}Clinical variables = Age, Severity

Table 14 shows DI-MS/MS Data of Day 4 samples relating to mortality outcome.

TABLE 14
DI-MS/MS data, Day 4 Samples, Mortality Outcome

| | | | | | | | | Variables | |
|---|---|---|---|---|---|---|---|---|---|
| | Method | Models | Q2 | R2Y | Sensitivity | Specificity | AUROC | (#) | Patients |
| DI- | PLS-DA | Metabolomics | 0.57 | 0.67 | 100 | 100 | 1 | 31ª | Died, |
| MS/MS | SIMPLS | Metabolomics | 0.21 | 0.41 | 93 | 94 | 1 | 31ª | n = 13 |
| Day 4 | | Clinical | 0.17 | 0.34 | 90 | 83 | 0.91 | 2^{b} | Alive, |
| Mortality | | Variables | | | | | | | n = 13 |
| outcome | | Combination | 0.22 | 0.43 | 93 | 100 | 0.96 | 31ª + 2^{b} | |
| | | metabolomics | | | | | | | |
| | | and clinical | | | | | | | |
| | | variables | | | | | | | |

^{a}Metabolites;
^{b}Clinical variables = Age, Severity

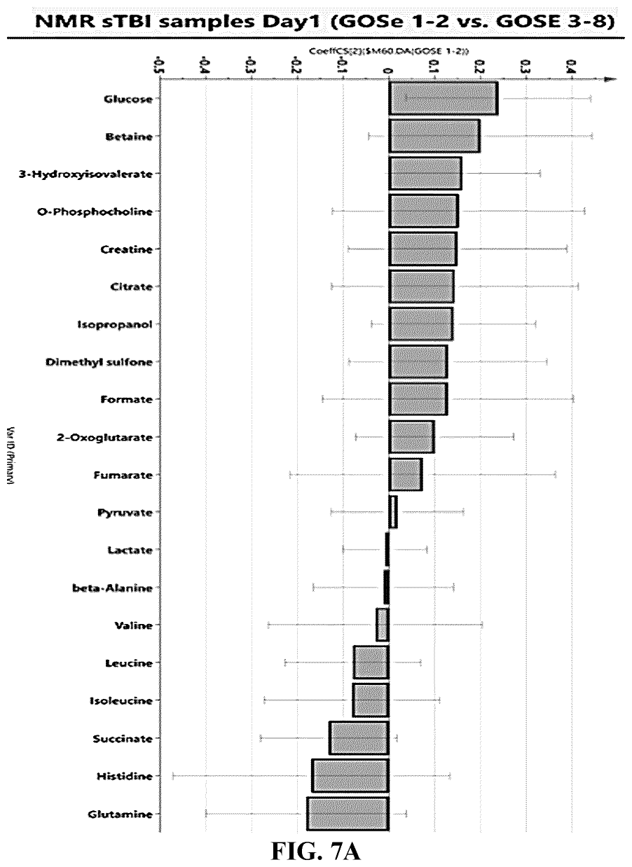

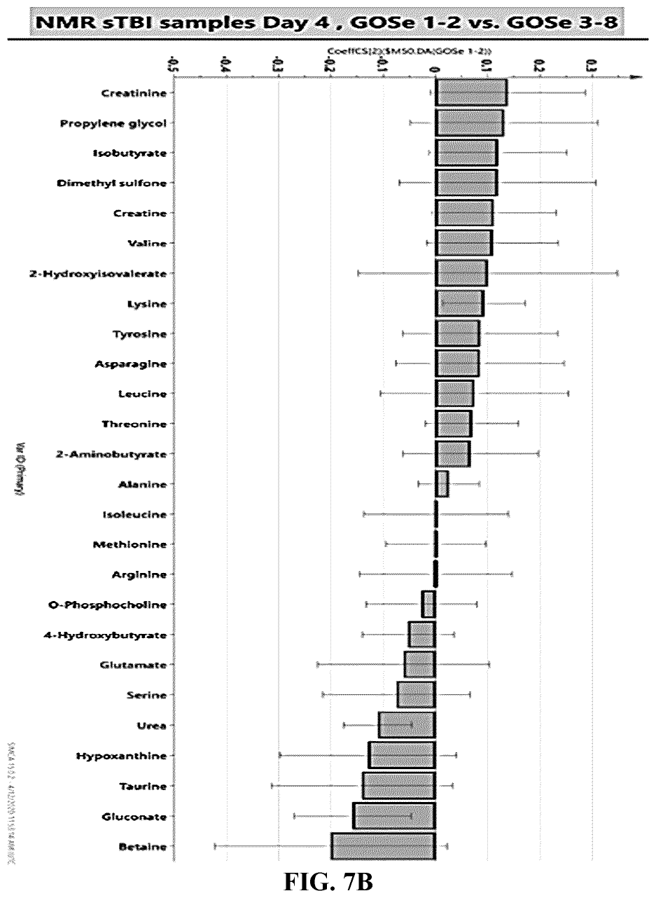

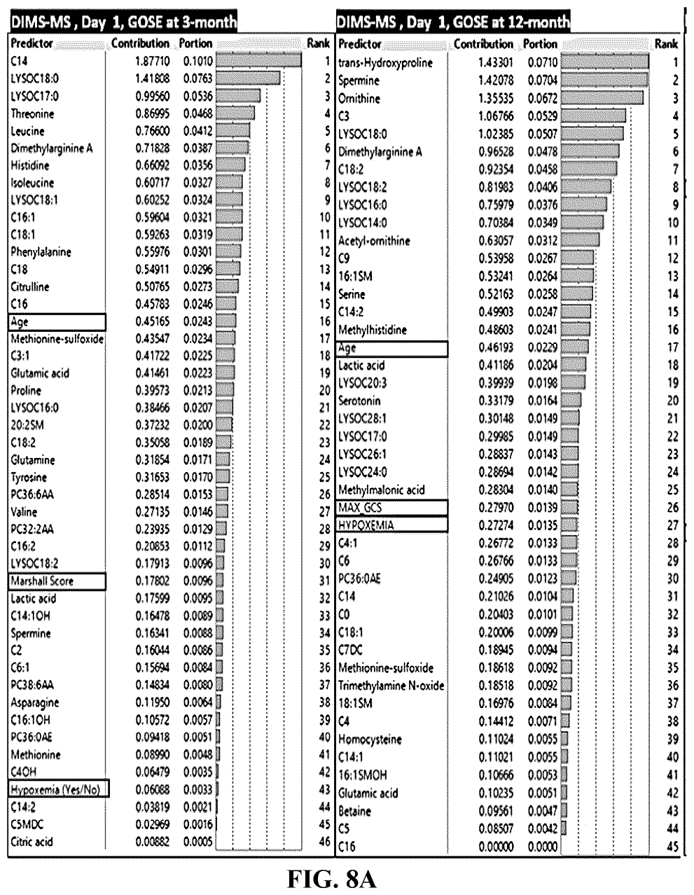

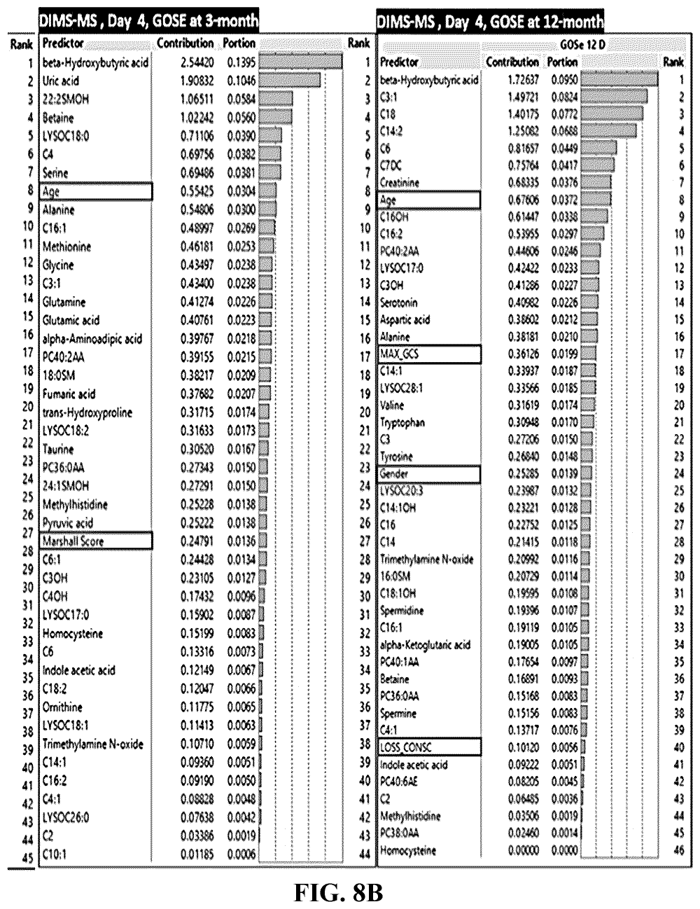

FIG. 7A and FIG. 7B show NMR data of prognosis of mortality and vegetative state for GOS-E level 1-2 versus GOS-E level 3-8 based on TBI Day 1 and Day 4 metabolites, respectively.

Table 15 shows NMR Data of Day 1 samples, and mortality outcome.

TABLE 15
NMR data, Day 1 Samples, Mortality Outcome

| | | | | | | | | Variables | |
|---|---|---|---|---|---|---|---|---|---|
| NMR | Method | Models | Q2 | R2Y | Sensitivity | Specificity | AUROC | (#) | Patients |
| Day 1 | PLS-DA | Metabolomics | 0.30 | 0.67 | 81 | 100 | 1 | 20ª | Died, |
| Mortality | SIMPLS | Metabolomics | 0.54 | 0.71 | 87 | 93 | 1 | 20ª | n = 21 |
| Outcome | | Clinical | 0.37 | 0.44 | 86 | 95 | 0.88 | 2^{b} | Alive |
| | | Variables | | | | | | | n = 23 |
| | | Combination | 0.67 | 0.73 | 81 | 95 | 0.91 | 20ª + 2^{b} | |
| | | metabolomics | | | | | | | |
| | | and clinical | | | | | | | |
| | | variables | | | | | | | |

^{a}Metabolites;
^{b}Clinical variables = Age, Severity

Table 16 shows NMR Data of Day 4 samples, and mortality outcome.

TABLE 16
NMR data, Day 4 Samples, Mortality Outcome

| | | | | | | | | Variables | |
|---|---|---|---|---|---|---|---|---|---|
| | Method | Models | Q2 | R2Y | Sensitivity | Specificity | AUROC | (#) | Patients |
| NMR | PLS-DA | Metabolomics | 0.47 | 0.62 | 88 | 94 | 0.94 | 17ª | Died, |
| Day 4 | SIMPLS | Metabolomics | 0.65 | 0.74 | 87 | 90 | 0.94 | 17ª | n = 12 |
| Mortality | | Clinical | 0.19 | 0.33 | 84 | 78 | 0.86 | 1^{b} | Alive |
| Outcome | | Variables | | | | | | | n = 19 |
| | | Combination | 0.68 | 0.75 | 84 | 98 | 0.93 | 17ª + 2^{b} | |
| | | metabolomics | | | | | | | |
| | | and clinical | | | | | | | |
| | | variables | | | | | | | |

^{a}Metabolites;
^{b}clinical variables = Age, Severity

As observed within the NMR data pertaining to prognosis of mortality and vegetative state (died vs alive) based on Day 1 serum, the primary increased metabolites include: glucose, betaine, O-phosphocholine, creatine, citrate, and dimethyl sulfone; and the primary decreased metabolites include: glutamine, histidine, succinate, isoleucine, leucine, and valine.

As observed within the NMR data pertaining to prognosis of mortality and vegetative state (died vs alive) based on Day 4 serum, the primary increased metabolites include: creatine, isobutyrate, dimethylsulfone, creatine, valine, tyrosine, asparagine, and tyrosine; and the primary decreased metabolites include: betaine, gluconate, taurine, hypoxanthine, urea, serine, and glutamate.

Table 17 shows the prediction of sTBI outcome at 12 months.

TABLE 17
The Prediction of sTBI Mortality at 3 Months

| | number of | | |
|---|---|---|---|
| | metabolites | Most important | |
| | for each set | metabolites | Name of Metabolites |
| The | 15 | Acylcarnitines (ACs) | C3:1, PC38:Oaa, PC40:6ae, Glucose, |
| minimum | | and | C7DC, Glutamine, Valine, Isoleucine, |
| set of | | Lysophosphatidylcholines | Leucine, C16OH, α-ketoglutarate, |
| Biomarkers | | (LysoPCs) | Hippurate, LysoPC26:0, Taurine |
| The middle | 30 | Acylcarnitines, | C3:1, PC38:Oaa, PC40:6ae, Glucose, |
| set of | | Lysophosphatidylcholines | C7DC, Glutamine, Valine, Isoleucine, |
| biomarkers | | and excitatory | Leucine, C16OH, α-ketoglutarate, |
| | | neurotransmitters such | Hippurate, LysoPC26:0, Taurine, |
| | | Glutamate, Tyrosine, | indole acetic acid, PC40:1aa, |
| | | Phenylalanine | Methylhistidine, α-aminoadipic acid, |
| | | | C10:1, arginine, Citrulline, C5MDS, |
| | | | Homocysteine, Homovanillic acid, |
| | | | C4:1, C14:1, C14, Succinate, C18:2 |
| The | 40 | Acylcarnitines (ACs), | C3:1, PC38:0aa, PC40:6ae, Glucose, |
| maximum | | Lysophosphatidylcholines | C7DC, Glutamine, Valine, Isoleucine, |
| set of | | (LysoPCs), excitatory | Leucine, C16OH, α-ketoglutarate, |
| biomarkers | | neurotransmitters such | Hippurate, LysoPC26:0, Taurine, |
| | | Glutamate, Tyrosine, | indole acetic acid, PC40: 1aa, |
| | | Phenylalanine, | Methylhistidine, α-aminoadipic acid, |
| | | Asparagine, | C10:1, arginine, Citrulline, C5MDS, |
| | | Phosphatidylcholines | Homocysteine, Homovanillic acid, |
| | | (PCs), Lactate, Pyruvate, | C4:1, C14:1, C14, Succinate, C18:2, |
| | | Citrulline, Ornithine, | C18:1, C12:1, C16:2, Tryosine, |
| | | Uric acid, Kynurenine | Threonine, C3, Valine. C8, Creatinine |

The metabolite pathway analysis involved in GOS-E prognosis at 12 months suggest that the energy metabolism pathways, excitotoxicity pathways, and acylcarnitine metabolism pathways (for example, with mitochondrial involvement) are impacted. The involvement of these pathways is affirmed if mortality as an outcome (instead of GOS-E good versus bad outcome) is assessed.

The metabolomics techniques conducted in this Example exhibited high efficacy in the prognosis of sTBI using GOS-E at short (3 months) and long term (12 months) intervals. Favorable (GOS-E 5-8) outcomes can be distinguished from unfavorable outcome (GOS-E 1-4) according to the platform methodologies described herein.

The metabolic biosignatures obtained by MS/MS analysis (including lipid compounds) showed excellent predictability for the prognosis of outcome. The 1H-NMR metabolite analysis also resulted in good predictability.

While both blood samples provided useful prediction, the metabolic biosignatures on day 4 post-injury were more predictive than day 1 post-injury samples.

Specifically, increased lysophosphatidylcholines, acylcarnitines, energy-related metabolites (glucose, lactate), aromatic amino acids and glutamate were positively correlated with poor outcome.

Metabolomic analysis using either MS/MS or NMR were effective predictors of the prognosis of mortality or vegetative state (GOS-E 1-2), as well as the assessment of severity of TBI (using GOS-E). QOLIBRI™ and PedsQL™ assessment (data not shown) provided useful parameters. A minimal number of metabolites may be assessed as biomarkers to build effective predictive models. Such models may be useful in making decisions regarding clinical care.

Table 18 shows the quantitative predictive values as determined using two different analytical platforms, including test sensitivity and specificity. In Table 18, the characteristics of the prediction models show a higher predictability of metabolic profiles on day 4 than day 1 post-sTBI for 3 and 12 month GOS-E and mortality at 3 months outcome. Additionally, the metabolic profiles obtained by MS/MS are more predictive than 1H-NMR results. The parameter “R2” indicates the goodness of fit of the model; “Q2” indicates the goodness of prediction of the model; and “AUC” represents the area under the receiver operating curve of the model.

TABLE 18
Quantitative Predictive Values

| | Analytical | Sampling | | | | | | |
|---|---|---|---|---|---|---|---|---|
| Prognosis | Platforms | Time | R² | Q² | p value | Sensitivity | Specificity | AUC |
| Poor vs. | DI-MS/MS | Day 1 | 0.74 | 0.39 | 1.5 × 10⁻³ | >99 | >99 | 0.99 |
| Good | | Day 4 | 0.81 | 0.61 | 5.4 × 10⁻⁵ | >99 | >99 | 0.99 |
| outcome | ¹H-NMR | Day 1 | 0.49 | 0.21 | 2.6 × 10⁻² | >99 | 87 | 0.99 |
| 3-month | | Day 4 | 0.75 | 0.52 | 6.0 × 10⁻³ | >99 | 90 | 0.99 |
| Poor vs. | DI-MS/MS | Day 1 | 0.83 | 0.55 | 8.0 × 10⁻⁴ | >99 | >99 | 0.99 |
| Good | | Day 4 | 0.80 | 0.63 | 1.5 × 10⁻⁴ | >99 | >99 | 0.99 |
| outcome | ¹H-NMR | Day 1 | 0.73 | 0.44 | 5.0 × 10⁻³ | 73 | 93 | 0.92 |
| 12-month | | Day 4 | 0.71 | 0.45 | 1.4 × 10⁻² | 94 | 92 | 0.95 |
| Mortality | DI-MS/MS | Day 1 | 0.72 | 0.49 | 4.2 × 10⁻⁵ | >99 | >99 | 0.99 |
| outcome | | Day 4 | 0.7 | 0.57 | 1.0 × 10⁻⁴ | >99 | >99 | 0.99 |
| | ¹H-NMR | Day 1 | 0.67 | 0.30 | 3.0 × 10⁻³ | 81 | >99 | 0.94 |
| | | Day 4 | 0.62 | 0.47 | 5.0 × 10⁻³ | 88 | 94 | 0.96 |

### Discussion

In the current example, serum-based metabolomics analysis was successfully applied on days 1 and 4 post sTBI of patients to predict GOSE outcomes at 3 and 12 months post-injury. Prediction models showed highly predictive and significant separation between sTBI patients with unfavorable and favorable outcomes. A remarkable similarity was found for the trends in changes in metabolites measured by both MS/MS and ¹H-NMR methodologies, showing a high level of reliability of quantification and validation of the results using two different analytical platforms. This study showed that the patients' demographics and clinical variables were not strong independent predictors of GOSE outcome. However, age, GCS, hypoxemia, injury severity score, and Marshall score revealed they can be used to slightly enhance the performance of metabolomics-based multivariate models for predicting outcomes. Conventional demographics and clinical features predominantly depend on the characteristics of the study cohort.

Although age¹⁶⁻¹⁸, GCS¹⁹, Marshall score¹⁹⁻²² and CT findings²¹ have had some value for predicting TBI outcome, it has been shown that conventional demographics, clinical variables and CT findings are overall insufficient predictors for the prognosis of outcome¹⁰.

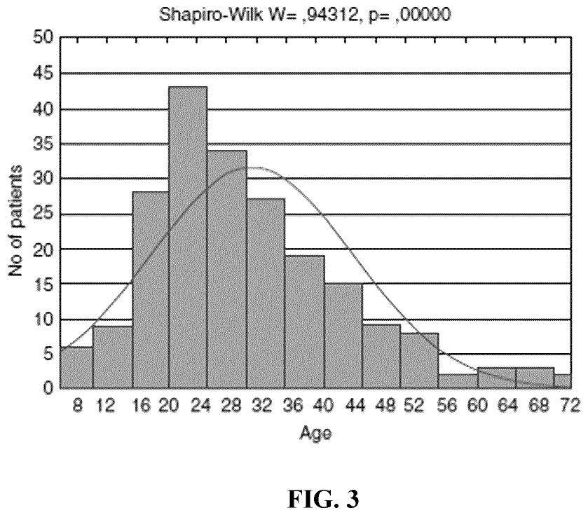

FIG. 3 illustrates typical patient age distribution of TBI, with the Shapiro-Wilk test for normality indicating W=0.94312 (p=0.00000).

CT scanning has been associated with improvement of prognostic value in patients with sTBI when combined with physiological findings²². Our results were similar to the IMPACT and CRASH studies²³ in their use of demographics and clinical features for predicting unfavorable outcomes and mortality of moderate to severe TBI at 6 months. The IMPACT and CRASH models were established based on age, GCS motor, pupillary reactivity, CT classification, EDH (epidural hematoma), tSAH (subarachnoid hemorrhage), hypoxia, and hypotension²³. In addition, using a population of the European Brain Injury Consortium Core Data (EBIC) and Traumatic Coma Data Bank (TCDB) studies, ²⁴multivariate analysis highlighted that age, GCS motor, pupillary reactivity, hypoxia, hypotension and CT classification were the most important predictors of outcomes (AUC 0.83-0.89). Age and injury severity score were shown as the most differentiating prognostic variables for mortality, while the IMPACT prediction model revealed age, GCS motor score, pupillary reactivity, hypoxia, hypotension, basal cisterns narrowing, midline shift and tSAH as the most predictive variables for 14-day mortality²⁵. Using a multimodal approach, physiological (ICP, MAP, CPP and pbtO2) and biochemical (pyruvate, lactate, glycine, glutamate, and glucose) parameters could predict the outcome in sTBI with a high degree of prediction accuracy around 90% 26. This study also demonstrated the importance of multivariate predictive and machine learning based-models versus simplified methods to determine the most differentiating metabolites and clinical variables as key predictors. It has previously been shown that using a Bayesian networks approach can improve the prediction models using variables that were not predictive in simplified models²⁷. PLS-DA and SIMPLS demonstrate the power of multivariate methods to explore big and complex datasets with many variables and relatively small sample sizes 28.

The present study also provides evidence for clinically and biologically relevant correlation of metabolite alterations to prognosticate sTBI outcome that provides mechanistic insight into the pathogenesis of sTBI. More specifically, increased lysoPC compounds in patients with the unfavorable outcome may be correlated with microvascular barrier disruption, promotion of oligodendrocyte demyelination and pericyte loss and induced inflammation²⁹. Increased C18 and its derivatives (stearic acid, oleic acid, linoleic acid) and lysoPCs in unfavorable outcome may correlate with docosahexaenoic acid (DHA) metabolism, a highly enriched lipid in the brain³⁰. It has previously been shown there is an increase of lysoPCs in CSF samples on day 1 post-injury in non-survivors and an increase of PCs in survivors from TBI 31 and in mild TBI patients compared to non-concussed controls³². Within one day post-sTBI, an increase of energy-related metabolites (lactate, glucose, and TCA cycle compounds) was observed in patients with unfavorable outcomes. The lactate/pyruvate ratio has been well-recognized as a predictor for the prognosis of brain injuries such as apoptosis, cerebral anoxia, and anaerobic metabolism^{33,34}. There is a correlation between elevated lactate with unfavorable outcomes in TBI, in association with reduced cerebral blood flow (CBF), elevated ICP, and ischemia^{33,34}. The increased tryptophan, kynurenine, tyrosine, phenylalanine, and glutamate on day 4 post-injury may intriguingly imply the correlation of excessive excitotoxicity mechanisms³⁵ and aromatic amino acids metabolism³⁶ with unfavorable outcome. Increased quinolinic acid, the final product of the tryptophan-kynurenine pathway, has been associated with the inflammatory response due to infiltration of macrophages and activated microglia in the CNS 37 and with unfavorable outcome and mortality in sTBI, indicating the possibility of the elevation of macrophage-derived (or microglia-derived) excitotoxin in the contribution of secondary injury to poor outcome^{37,38}. In our study, elevated kynurenine, and tryptophan in patients with an unfavorable outcome on day 4 after injury may characterize excessive neuroinflammation, a well-known secondary injury mechanism in brain injury. Also increased NAA and phenylalanine mainly on day 4 post sTBI, two well-known neurotransmitters in patients with unfavorable outcomes, may be associated with alteration of osmolality and catecholaminergic mechanism of injury³⁹. The current data also showed the association of day 1 hyperglycemia and increased lactate with poor outcome. Hyperglycemia and hyperlactatemia have been previously shown to be potential predictors for the prognosis of unfavorable TBI outcome⁴⁰⁻⁴².

There are important limitations to this study, in particular, there is a relatively small sample size in general and there are a small number of patients with outcome measures missing at different time points. In addition, the study is noted to be male-dominated, a common problem when studying sTBI in adults. These limitations, therefore, require that this study be validated using a larger cohort of adults with sTBI. Nonetheless, this study does show great promise in using metabolomics to evaluate sTBI, particularly in prognosis assessment.

Metabolic profiling of sTBI patient samples for a longer period than the first 4 days may enhance the predictability of metabolomics for the prognosis of outcome and may provide more definitive information about molecular changes post sTBI, especially in those who have a favorable outcome of sTBI. Also, applying an untargeted mass spectrometry approach may help identify more known and unknown metabolites that may be correlated with the prognosis of sTBI and may more clearly define the mechanisms of injury (both primary and secondary) in sTBI.

Despite the study limitations, it can be concluded that multiple metabolite alterations are detectable in serum due to sTBI early in the injury process. In the current study, metabolite alterations on days 1 and 4 post-sTBI were well-correlated with the GOSE unfavorable and favorable outcomes at 3 and 12 months and importantly, may be used as a promising prognostic tool for predicting the worst GOSE outcome, i.e., death post-injury. Metabolomics appears to be superior to patients' demographics and clinical features in predicting GOSE outcome at 3 and 12 months post injury. Notably, the combination of metabolomics with clinical and CT variables can enhance the prognostication of sTBI in the early days post-injury. Importantly, the information derived from metabolomics and prediction models may be used for the stratification of patients with sTBI that can be applied in future clinical trials, especially therapeutic trials as a means of prognostic enrichment. Targeted DI-MS/MS (including multiple lipid metabolites) appears to be superior to 1H-NMR for predicting sTBI outcome and this information may be useful for future studies.

### Example 1A

Metabolomic Profiles in Serum Predict Global Functional Neurological Outcome at 3 and 12 Months and Death at 3 Months Following sTBI

### SUMMARY

Prognostication is very important to clinicians and families during the early management of severe traumatic brain injury (sTBI). However, there are no clinically reliable biomarkers to determine prognosis in sTBI. As has been demonstrated in several diseases, early measurement of serum metabolomic profiles can be used as sensitive and specific biomarkers to predict outcome.

Methods. Data from Example 1 is further analysed and elaborated upon in this Example 1A. Adults with sTBI (Glasgow coma scale≤8) were prospectively enrolled in a multicenter CanTBI study. Serum samples were drawn on the 1st and 4th day following injury for metabolomic profiling. The Glasgow outcome scale extended (GOSE) was collected at 3 and 12 months post-injury. Targeted direct infusion liquid chromatography tandem mass spectrometry (DI/LC-MS/MS or simply “MS/MS” herein) and untargeted proton nuclear magnetic resonance spectroscopy (1H-NMR) were used to profile serum metabolites. Multivariate analysis was used to determine the association between serum metabolomics and GOSE, dichotomized into favorable and unfavorable, outcomes.

Findings. Serum metabolic profiles on days 1 and 4 post-injury were highly predictive (Q²>0.4-0.5) and highly accurate (AUC>0.99) to predict GOSE outcome at 3 and 12 months post-injury and mortality at 3 months. The metabolic profiles on day 4 were more predictive (Q²>0.55) than those measured on day 1 post-injury. Increased lysophosphatidylcholines, acylcarnitines, energy-related metabolites (glucose, lactate), aromatic amino acids and glutamate were associated with poor outcome and mortality.

Interpretation. Metabolomic profiles were strongly associated with prognosis of GOSE outcome at 3 and 12 months and mortality following sTBI in adults. The current findings strongly support the use of serum metabolomics which are shown to be better than clinical data in determining prognosis in adults with sTBI in the early days post-injury. Our findings, however, require validation in a larger cohort of adults with sTBI before using in clinical practice.

### Introduction

Traumatic brain injury (TBI) is a neurologic injury resulting from an external mechanical force and one of the most common causes of long-term neurological disability and death.¹ Worldwide, approximately 69 million people suffer TBI annually.² There are 5.3 and 7.7 million individuals living with TBI-related disability in the United States and European countries¹, respectively. Severe TBI has a mortality of 30-50% and 30% of survivors have severe neurologic sequelae.³⁻⁷ Large variability in the mechanisms of TBI, patterns of brain injury and a large range of outcomes make it difficult to determine prognosis in the first few days following TBI.⁸ Clinical factors and neuroimaging findings are not reliable predictors of outcomes following TBI.^{9,10} Blood biomarkers have the potential to improve prognostic models. These models could help clinicians during discussions with surrogate decision-makers about the intensity of acute care and help plan rehabilitation and support services for survivors and their caregivers. Metabolomics is widely used to provide potential insights into mechanisms of injury and may allow the development of sensitive and specific biomarkers for these prognostic models.¹¹

In this study, it was hypothesized that serum metabolites would be associated with favorable and unfavorable outcomes at 3 and 12 months following severe TBI. The objectives were to measure metabolites in serum sampled at 1 and 4 days following severe TBI, to determine concentration thresholds for prognosis and to compare prognostic models using metabolomics biomarkers to models using clinical predictors.

### Materials and Methods

Patients' characteristics and primary clinical information. Patients≥18 years old with severe TBI (Glasgow coma scale≤8) were enrolled prospectively at 3 hospitals in Vancouver, Calgary and Halifax, Canada. Serum samples were collected on days 1 and 4 post-injury. Demographics, injury characteristic, neuroimaging (CT scan) and physiologic clinical variables were collected electronically as well as global neurological function and mortality at 3 and 12 months following injury using the Glasgow Outcome Scale-Extended (GOSE). All data was collected and cleaned by trained research coordinators and database engineers. The GOSE was dichotomized into favorable (GOSE 5-8) and unfavorable (GOSE 1-4) outcomes. The collected clinical variables in this study included gender, age, GCS, ISS (injury severity score), intubation, hypoxemia, hypotension, loss of consciousness and Marshall score that were used for the prediction of GOSE outcome at 3 and 12 months, and for mortality at 3 months.

A total of 445 adult and pediatric patients with mild, moderate and severe forms of TBI were entered into the CanTBI study and database. All patients were admitted to critical care units, and/or emergency departments and/or assessed in concussion clinics at participating centers. There were both pediatric and adult arms to the CanTBI study. In the adult arm, the inclusion criteria for adults severe sTBI included:
1. Patient≥18 yrs with acute mild, moderate or severe TBI.
2. Patient had at least 1 research blood sample drawn within 24 hours+6 hours from TBI.
3. Patient/substitute decision maker can speak and read English and/or French.
4. Patient/substitute decision maker has a fixed address.
5. Obtaining informed consent from patient or designated legal surrogate either directly or in a delayed fashion.

Exclusion criteria consisted of:
1. Patient had a severe neurodevelopmental disorder pre-injury.
2. Patient has a confirmed or suspected brain death at the time of enrollment determined by the attending physician.
3. Patient has a terminal illness, expected to live less than 12 months from TBI.
4. Patient has ongoing neurologic deficit from a previous TBI or other acquired brain injury (e.g. stroke).
5. Patient had a cardiac event that potentially caused a TBI.
6. Patient/substitute decision maker is unwilling to participate in study follow-up.

Biological samples including whole blood, serum, plasma, buffy coat, CSF and brain material from any biopsy or from operative procedure that were collected in the CanTBI study at various dates and times over 28 days (as per the CanTBI Protocol). All biological samples were collected and handled as per predefined CanTBI SOPs with the goal of sample collection to freezer within 2 hours. Demographic and clinical data were collected from individuals including age at the time of TBI, sex, cause of TBI, pre-hospital events, GCS score, Abbreviated Injury Score (AIS), Injury severity score (ISS), clinical monitoring, medication and medical interventions.

Extra information such as socioeconomic status, education and past medical history (prior concussion, migraine, psychiatric history, neurological history) were collected and assessed by an expert team for relevance. Details of lab and neuroimaging and neurophysiology were documented. Entered patients in the study participated in a battery of questionnaires and performance-based cognitive and behavioral assessments that focused on global outcome, TBI-related symptoms and quality of life at time points predetermined based on the severity of the injury. Glasgow Outcome Scale Extended (GOSE)/Extended Pediatric (GOS-EP) and Extended Pee Wee GOS (GOS-E P-WEE) for pediatric patients, Rivermead Post Concussion Symptom Questionnaire (RPSQ), Brief Test of Adult Cognition (BTACT), Pediatric Quality of Life Questionnaire (PedsQL™), Health Behavior Inventory (HBI), and Patient-Reported Outcomes Measurement Information System (PROMIS) were the primary and supplementary TBI outcomes collected from patients. This study focused on GOSE outcome at 3 and 12 months post injury and mortality at 3 months.

Metabolomics Methods and Quantification. Untargeted proton nuclear magnetic resonance (¹H-NMR) spectroscopy and targeted direct injection, liquid chromatography tandem mass spectrometry (DI/LC-MS/MS) were applied to identify and quantify serum metabolites on days 1 and 4 post sTBI. These two techniques were used to quantify a broad list of metabolites with few overlapping metabolites. A comprehensive targeted analysis of 130 and 58 metabolites was carried out using DI/LC-MS/MS and ¹H-NMR, respectively, to determine serum metabolites on days 1 and 4 post-injury.

Direct infusion/liquid chromatography tandem mass spectrometry (DI/LC-MS/MS). Targeted, quantitative DI/LC-MS/MS was performed on days 1 and 4 post-sTBI serum samples using an ABI 4000 Q-Trap (Applied Biosystems/MDS Sciex) mass spectrometer. A targeted list of metabolites used in this study consisted of 130 metabolites including lipids, amino acids, biogenic amines and organic acids plus other metabolites.

Table 19 provides a list of quantified metabolites. Reverse-phase liquid chromatography-tandem Mass Spectrometry (LC-MS/MS) was used to quantify amino acids, biogenic amines, and organic acids. Direct infusion tandem mass spectrometry (DI-MS/MS) was applied to quantify glycerophospholipids (lysophosphatidylcholines (lysoPCs) and phosphatidylcholines (PCs), acylcarnitines (Cs), and sphingomyelins (SMs).

TABLE 19
Metabolites quantified using DI/LC-MS/MS

| | Metabolite |
|---|---|
| 1 | Asymmetric dimethylargir |
| 2 | total dimethylarginine |
| 3 | alpha-Aminoadipicacid |
| 4 | Creatinine |
| 5 | Dopamine |
| 6 | Kynurenine |
| 7 | Methioninesulfoxide |
| 8 | Hydroxyproline (t4-OH-Pr |
| 9 | Phenylethylamine |
| 10 | Putrescine |
| 11 | Sarcosine |
| 12 | Serotonin |
| 13 | Spermidine |
| 14 | Spermine |
| 15 | Taurine |
| 16 | Tyramine |
| 17 | Alanine |
| 18 | Arginine |
| 19 | Asparagine |
| 20 | Aspartate |
| 21 | Citrulline |
| 22 | Glutamate |
| 23 | Glutamine |
| 24 | Glycine |
| 25 | Histidine |
| 26 | Isoleucine |
| 27 | Leucine |
| 28 | Lysine |
| 29 | Methionine |
| 30 | Ornithine |
| 31 | Phenylalanine |
| 32 | Proline |
| 33 | Serine |
| 34 | Threonine |
| 35 | Tryptophan |
| 36 | Tyrosine |
| 37 | Valine |
| 38 | Betaine |
| 39 | Choline |
| 40 | Creatine |
| 41 | Methylhistidine |
| 42 | Homocysteine |
| 43 | CO (Carnitine) |
| 44 | C2 (Acetylcarnitine) |
| 45 | C3:1 (Propenoylcarnitine) |
| 46 | C3 (Propionylcarnitine) |
| 47 | C4:1 (Butenylcarnitine) |
| 48 | C4 (butyrylcarnitine) |
| 49 | C3-OH (hydroxyPropionylcarnitine) |
| 50 | C5:1 (Tiglylcarnitine) |
| 51 | C5 (Valerylcarnitine) |
| 52 | C4-OH (C3-DC) (Hydroxybutyrylcarnitine) |
| 53 | C6:1 (Hexenoylcarnitine) |
| 54 | C6 (C4:1-DC) (Hexanoylcarnitine) |
| 55 | C5-OH (C3-DC-M) (hydroxyvalerylcarnitine) |
| 56 | C5:1-DC (Glutaconylcarnitine) |
| 57 | C5-DC (C6-OH)(Glutarylcarnitine) |
| 58 | C5 (Octanoylcarnitine) |
| 59 | C5-M-DC (methylglutarylcarnitine) |
| 60 | C9 (Nonaylcarnitine) |
| 61 | C7-OC (pimelylcarnitine) |
| 62 | C10:2 (decadienylcarnitine) |
| 63 | C10:1 (Decenoylcarnitine) |
| 64 | C10 (Decanoylcarnitine) |
| 65 | C12:1 (Dodecenoylcarnitine) |
| 66 | C12 (dodecanoylcarnitine) |
| 67 | C14:2 (Tetradecadienylcarnitine) |
| 68 | C14:1 (tetradecenoyl carnitine) |
| 69 | C14 (tetradecanoylcarnitine) |
| 70 | C12-DC (dodecanedioylcarnitine) |
| 71 | C14:2-OH (hydroxytetradecadienylcarnitine) |
| 72 | C14:1-OH (Hydroxytetradecenoyl carnitine) |
| 73 | C16:2 (Hexadecadienylcarnitine) |
| 74 | C16:1 (Hexadecenoylcarnitine) |
| 75 | C16 (Hexadecanoylcarnitine) |
| 75 | C16:1-OH (Hydroxyhexadecenoylcarnitine) |
| 77 | C16-OH (hydroxyhexadecanoylcarnitine) |
| 78 | C18:2 (Octadecadienylcarnitine) |
| 79 | C18:1 (Octadecenoylcarnitine) |
| 80 | C18 (Octadecanoylcarnitine) |
| 81 | C18:1-OH (Hydroxyoctadecenoylcarnitine) |
| 82 | PC diacyl (aa) C36:6 |
| 83 | PC aa C32:2 |
| 84 | PC aa C38:0 |
| 85 | PC aa C38:6 |
| 86 | PC aa C40:1 |
| 87 | PC aa C40:2 |
| 88 | PC aa C40:6 |
| 89 | PC acyl-alkyl (ae) C40:5 |
| 90 | PC ae C36:0 |
| 91 | SM (OH) C14:1 |
| 92 | SM (OH) C16:1 |
| 93 | SM (OH) C22:1 |
| 94 | SM (OH) C22:2 |
| 95 | SM (OH) C24:1 |
| 96 | SM C16:0 |
| 97 | SM C16:1 |
| 98 | SM C18:0 |
| 99 | SM C18:1 |
| 100 | SM C20:2 |
| 101 | lysoPC a C14:0 |
| 102 | lysoPC a C16:0 |
| 103 | lysoPC a C16:1 |
| 104 | lysoPC a C17:0 |
| 105 | lysoPC a C18:0 |
| 106 | lysoPC a C18:1 |
| 107 | tysoPC a C18:2 |
| 108 | lysoPC a C20:3 |
| 109 | lysoPC a C20:4 |
| 110 | lysoPC a C24:0 |
| 111 | lysoPC a C26:0 |
| 112 | lysoPC a C26:1 |
| 113 | lysoPC a C28:0 |
| 114 | tysoPC a C28:1 |
| 115 | Lactic acid |
| 116 | beta-Hydroxybutyric acid |
| 117 | alpha-Ketoglutaric acid |
| 118 | Citric acid |
| 119 | Butyric acid |
| 120 | HPHPA |
| 121 | para-hydroxyhippuric acid |
| 122 | Succinic acid |
| 123 | Fumaric acid |
| 124 | Pyruvic acid |
| 125 | Isobutyric acid |
| 126 | Hippuric acid |
| 127 | Methylmalonic acid |
| 128 | Homovanillic acid |
| 129 | Indole acetic acid |
| 130 | Uric acid |

To quantify organic acids, 150 μl of ice-cold methanol was added to thawed 50 μl serum samples followed by adding 10 μl of isotope-labelled standards. To precipitate proteins, the mixtures were kept in −20° C. overnight, and were centrifuged at 13,000×g for 20 min. 50 μl of supernatant extracts were added to a 96-well plate followed by adding 3-nitrophenylhydrazine reagent and were incubated for 2 hours. Before LC-MS/MS, 2 mg/ml of Butylated hydroxytoluene was added to the extract.

To quantify amino acids and lipids, 10 μl of samples were added to a 96-well plate and samples were dried using a nitrogen stream. Phenyl-isothiocyanate reagent was used to derivatize the compounds. Samples in the plate were incubated and dried using an evaporator. Extraction solvent (300 μl) was added to the samples followed by centrifugation to drive the analytes to the lower part of the 96-well plate. Formic acid (0.2%) in water and formic acid (0.2%) in acetonitrile was used in dilution. The isotope-labeled internal standards and other standards were used to quantify each metabolite in the list using multiple reaction monitoring (MRM) pairs.

For LC-MS/MS analyses, chromatography was performed using an Agilent reversed-phase Zorbax Eclipse XDB C18 column (3.0 mm×100 mm, 3.5 μm particle size, 80 A pore size) with a Phenomenex (Torrance, CA, USA) Security Guard C18 pre-column (4.0 mm×3.0 mm) was used to quantify the amino acids and biogenic amines. The parameter for chromatography was set up as follows: mobile phase A was 0.2% (v/v) formic acid in the water, and mobile phase B was 0.2% (v/v) formic acid in acetonitrile. The gradient parameters were t=0 min, 0% B; t=0.5 min, 0% B; t=5.5 min, 95% B; t=6.5 min, 95% B; t=7.0 min, 0% B; and t=9.5 min, 0% B. The chromatography column was set as 50° C. 10 μl of samples were injected into the column with the flow rate at 300 μl/min. For organic acid chromatography was set up as follows: mobile phase A was 0.01% (v/v) formic acid in the water, and mobile phase B was 0.01% (v/v) formic acid in methanol. The gradient parameters were t=0 min, 30% B; t=2.0 min, 50% B; t=12.5 min, 95% B; t=12.5 min, 100% B; t=13.5 min, 100% B; and t=13.6 min, and finally 30% B for 4.4 min. The column was set at 40° C. 10 μl of samples were injected into the column with flow rate at 300 μl/min.

For DI-MS/MS analyses, samples were directly injected into the mass analyzer from the autosampler. The mobile phase was set by mixing 60 μl of formic acid, 10 ml of water and 290 ml of methanol. The flow rate was t=0 min, 30 μl/min; t=1.6 min, 30 μl/min; t=2.4 min, 200 μl/min; t=2.8 min, 200 μl/min and t=3.0 min, 30 μl/min. 20 μl of samples were injected into MS. The lipid concentration was measured semi-quantitatively using a single point calibration of representative metabolites obtaining by a linear regression.

For quantification of metabolites, standard calibration (seven points) was obtained for each of the organic acids, amino acids, and biogenic amines. The signal ion intensity of metabolites was corrected to the corresponding internal standards followed by calculating the concentration using the quadric regression with a 1/x² weighting

The first row of the plate is devoted to 1 blank, 3 zero samples, 1 standard, and 3 quality controls. The samples were delivered to the mass spectrometer using direct infusion.⁴⁷ MetIQ software was used to control the entire assay workflow, from sample registration to automated calculation of metabolite concentrations to the export of data into other data analysis programs. A targeted profiling scheme was used to quantitatively screen for known small molecule metabolites using multiple reaction monitoring, neutral loss, and precursor ion scans. Analyst 1.6.2 and MulitQuant 3.0.3 software was used for the quantification of metabolites concentration. Further details are previously described.⁴⁷

Proton Nuclear magnetic resonance spectroscopy. Untargeted one dimensional (1D) 1H-NMR spectroscopy was used to identify and quantify the serum metabolites on days 1 and 4 post-sTBI samples using a 600 MHz Bruker Ultrashield Plus NMR spectrometer (Bruker BioSpin Ltd., Canada) at the University of Calgary. To extract the metabolites, 200 μl of serum from each patient sample was ultrafiltered using 3 KDa NanoSep microcentrifuge filters that filters small molecules<3 KDa for analysis. DSS (4,4-dimethyl-4-silapentane-1-sulfonic acid) was used as an internal reference compound to quantify individual compounds⁴³. 10 μl sodium azide (NaN3) was added to all filtrates to inhibit bacteria growth in the sample. The final volume of samples was adjusted to 400 μl by adding D20 followed by adjusting pH to 7.0±0.04 at room temperature⁴⁴. The NMR spectroscopy was obtained in 1D spectra using the pre-saturation pulse sequence (noesypr1d) with an optimal water suppression program and a mixing time of 100 milliseconds (ms).^{44,45} NMR acquisition was shimmed and calibrated based on the DSS peak at 0.0 ppm when the half-height line width of the DSS peak was less than 1.5 Hz. NMR spectra were obtained using 1028 scans then zero-filled and Fourier transformed to 128K points. All NMR spectra were corrected for line broadening, phasing, baseline correction based on the DSS peak at 0.0 ppm using a Topspin software program (Bruker BioSpin Ltd., Canada)⁴⁶. ChenomX NMR Suite 7.1 software (ChenomX Inc., Edmonton, Alberta, Canada) was used to process and profile the NMR spectra for the identification and quantification of metabolites. In the processing module, NMR spectra were manually phased followed by baseline correction, and the water peak region was removed from the spectra. Processed NMR spectra were then transferred to the profiler module. Untargeted profiling was performed in a semi-manual approach to quantify metabolites based on the DSS concentration at mM or mg/dl. All spectra were randomly ordered for untargeted profiling to avoid progressive or systematic bias. Spectroscopy analysis has been described previously.⁴⁷

Table 20 provides a list of metabolites quantified by ¹H-NMR.

TABLE 20
Metabolites Quantified Using ¹H-NMR

| | Metabolite |
|---|---|
| 1 | 2-Aminobutyrate |
| 2 | 2-Hydroxybutyrate |
| 3 | 2-Hydroxyisovalerate |
| 4 | 2-Oxoglutarate |
| 5 | 2-Oxoisocaproate |
| 6 | 3-Hydroxybutyrate |
| 7 | 3-Hydroxyisovalerate |
| 8 | 3-Methyl-2-oxovalerate |
| 9 | 4-Hydroxybutyrate |
| 10 | Acetate |
| 11 | Acetoacetate |
| 12 | Acetone |
| 13 | Adipate |
| 14 | Alanine |
| 15 | Arginine |
| 16 | Asparagine |
| 17 | Aspartate |
| 18 | Betaine |
| 19 | Carnitine |
| 20 | Choline |
| 21 | Citrate |
| 22 | Creatine |
| 23 | Creatinine |
| 24 | Dimethyl sulfone |
| 25 | Dimethylamine |
| 26 | Formate |
| 27 | Fumarate |
| 28 | Gluconate |
| 29 | Glucose |
| 30 | Glutamate |
| 31 | Glutamine |
| 32 | Glycerol |
| 33 | Glycine |
| 34 | Histidine |
| 35 | Hypoxanthine |
| 36 | Isobutyrate |
| 37 | Isoleucine |
| 38 | Isopropanol |
| 39 | Lactate |
| 40 | Leucine |
| 41 | Lysine |
| 42 | Mannose |
| 43 | Methionine |
| 44 | N-Acetylaspartate |
| 45 | N-Acetyltyrosine |
| 46 | O-Phosphocholine |
| 47 | Ornithine |
| 48 | Phenylalanine |
| 49 | Proline |
| 50 | Pyruvate |
| 51 | Serine |
| 52 | Succinate |
| 53 | Taurine |
| 54 | Threonine |
| 55 | Tyrosine |
| 56 | Urea |
| 57 | Valine |
| 58 | Beta-Alanine |

Data Analysis. PCA was performed initially to find the trends, similarity, clustering and outliers (technical and biological outliers). PCA was performed as an unsupervised analysis to examine the metabolomics data before applying supervised analyses including partial least square discriminant analysis (PLS-DA), statistically inspired modification of partial least squares analysis (SIMPLS) and artificial neural network (ANN) analysis.

For MVA, the sensitivity, specificity, and AUC were calculated for PLS-DA models using prediction analysis and multivariate misclassification. Following a standard protocol, the prediction models were selected and verified based on performance parameters R²Y (or R2, goodness of fit), Q²Y (or Q2, goodness of prediction) and p value (level of significance) through a cross-validation (CV) method. CV was performed based on the leave-one-out cross validation (LOOCV) to assess generalizability of the results using an independent data set. These parameters are assigned for assessing the reliability, predictability and significance level of a model.⁴⁸ The prediction models were built using the most differentiating metabolites based on a variable importance in projection (VIP) level>1.0. Additionally, in order to minimize the metabolites in a model but still be predictable, the best prediction models were selected according to the criteria including the highest Q², with a significant p-value, with sensitivity and specificity>85% and with an AUC>0.90. This approach did not change the topmost important metabolites but dropped the less important metabolites in the prediction models. Permutation tests were performed using 200-times testing and this was applied to each prediction model to verify the Q2 value and help ensure the data was not overfit. Coefficient plots were applied to illustrate the most differentiating metabolites obtained by the prediction models (PLS-DA). The Coefficient plot, by default, displays the coefficients referring to scaled and centered data for a given response, with 95% confidence intervals derived from jack-knifing. Statistically inspired modification of PLS (SIMPLS), an alternative approach to PLS regression⁴⁹, was performed to build prediction models using clinical data and for the combination of clinical and metabolite variables. Also, artificial neural network (ANN) and predictor screening analysis were applied to extract more information from metabolomics datasets and clinical variables as well as internal validation of prediction models obtained by PLS-DA and SIMPLS. ANN, as a supervised nonlinear approach, was used to classify metabolomics data particularly for model data where the relations or functions are not known. In this study, ANN was a suitable complementary method to PLS analysis due to identification of a subset of the variables with maximal explanatory power. ANN provided an interpretable description of biological data using prediction models obtained by training and validation subsets.⁵⁰ ANN was performed through launching two types of prediction models: training, and validation models using the most differentiating metabolites (VIP>1.0) obtained by PLS. Partition analysis (PA) was performed to find the relationship between the clinical variables and GOSE outcomes at 3 and 12 months. The algorithm of PA finds all possible splits of the clinical variables to best predict GOSE outcomes. PA can classify the patients using cutoff points of each clinical variable with either continuous or ordinal values. Cross-validation ANOVA (CV-ANOVA) and permutation test (200 times) analyses were performed as internal validation and to verify the predictability of the models.

Statistical Analysis. Multivariate analysis and machine learning were used to determine which serum metabolites were associated with favorable versus unfavorable Glasgow outcome scale extended (GOSE) outcomes. Principal component analysis (PCA) was used as a multi-variable analysis method to examine the variability and trends of metabolic profiles and to detect outliers. Partial least squares discriminant analysis (PLS-DA), a type of machine learning, was used to build prognostic models. Prognostic models were created using the most differentiating metabolites with a variable important of the projection (VIP) value>1.0. SIMCA-P v15.0.2 (Sartorius Stedim Biotech, Umea, Sweden) was used for the PLS-DA analysis. It was further analyzed whether clinical predictors or combining clinical predictors with metabolomics data yielded a superior model compared to metabolomics alone in predicting GOSE outcome. Statistically inspired modification of partial least squares (SIMPLS), an algorithm of the PLS method suitable for both nominal or continuous variables, was used to develop prediction models using only clinical predictors or combined clinical with metabolites variables for outcomes at 3 months, 12 months and for mortality at 3 months. Developed prognostication models were characterized by the metrics R2 (goodness of model fit), Q² (goodness of prediction), cross-validation p-value and permutation testing (200 times). Artificial Neural Network analysis (ANN) was performed to predict one response variable (unfavorable and/or favorable separately) using a flexible function of input variables. JMP Pro 14.3.0 (SAS Institute Inc. USA) was used for SIMPLS and ANN analysis. MetaboAnalyst 4.0 (available at www.metaboanalyst.ca) was used for multivariate and univariate analysis. Area under the receiver operating curve (AUC), sensitivity, and specificity were obtained using a multivariate approach.

To build prognostic models of outcome using clinical factors univariate analysis was first used, followed by multivariable analysis and generated AUC. Clinical factors with a P>0.05 from the univariate analysis were included in the multi-variable models.

### Results

Patient Characteristics. A total of 8239 patients were screened in the CanTBI study; 3465 patients screened positive for TBI (42%). After informed consent, 466 adult and pediatric patients with mild, moderate, and severe TBI were enrolled into the prospective CanTBI biobank and database for TBI study. There were 300 adult patients with TBI and 59 of these patients (19.6%) were diagnosed with severe TBI (sTBI) and included in this study.

Table 1 of Example 1 provides detailed patient and injury characteristics. Patients' characteristics, clinical information, GCS at admission, GOSE outcome distribution, CT findings, and Marshall score. * Shows the number of patients with the clinical information and the percentage of total patients, others included without clinical information, and missing information. ** the number of patients (percentage of total) were included in the same GCS categorized level. ‡ the number of patients with GOSE data at the same time. ‡ ‡ the number of patients that had the same CT findings; the rest may include patients without CT findings or findings missing in the study.

These 59 patients had a mean age of 50 years±20.6 (SD). FIG. 1 of Example 1 shows the patient flow chart with the numbers of patients with follow-up data at 3 and 12 months post-injury.

Table 21 shows the clinical prognostic model results for each clinical variable. Patient's demographics and clinical characteristics for unfavorable (GOSE 1-4) and favorable (GOSE 5-8) outcome groups at 3 and 12 months. * The variables are based on the number of patients. * These data included several variables that have not been shown in detail for each cohort. There was no significant difference for any type and location of injury between cohorts with favorable and unfavorable outcome at 3 and 12 months post injury.

TABLE 21
Clinical Variables

| Prediction of GOSE | 3 Month | | | 12 Month | | |
|---|---|---|---|---|---|---|
| | Poor | Good | | | Good | |
| Patients Characteristics | Outcome | Outcome | | Poor Outcome | Outcome | |
| and clinical information | (n = 35) | (n = 9) | p value | (n = 14) | (n = 15) | p value |
| Sex (Male/Female) | 30/5 | 6/3 | 0.42 | 11/3 | 13/2 | 0.82 |
| Age (mean ± SD) | 55.4 ± 20.4 | 40.5 ± 21.0 | 0.03 | 52.0 ± 18.7 | 38 ± 19.8 | 0.06 |
| Weight (mean ± SD) | 88.5 ± 19.5 | 76.4 ± 21.1 | 0.08 | 81.7 ± 22.6 | 79.3 ± 16.1 | 0.75 |
| Injury Severity Score (ISS) | 56.4 ± 22.6 | 35.1 ± 12.6 | <0.01 | 35.5 ± 12.5 | 36.4 ± 12.5 | 0.81 |
| (mean ± SD) | | | | | | |
| Admission-type | 13 (37.1%) | 2 (22.2%) | 0.36 | 4 (28.5%) | 4 (26.6%) | 0.58 |
| ER | 21 (60%) | 7 (77.7%) | | 10 (71.4%) | 11 (73.3%) | |
| ICU | | | | | | |
| Hypoxia (Yes/No)* | 8/22 | 0/9 | 0.07 | 3/8 | 1/14 | 0.38 |
| Intubated (Yes/No)* | 21/13 | 7/2 | 0.61 | 11/3 | 10/5 | 0.77 |
| Hypotension (Yes/No)* | 5/25 | 1/7 | 0.98 | 2/10 | 2/13 | 0.64 |
| Paralytic-AGT (Yes/No)* | 16/17 | 6/1 | 0.40 | 6/7 | 9/4 | 0.32 |
| Loss Consciousness* | 25/4 | 5/2 | 0.30 | 13/0 | 8/2 | 0.48 |
| Location of Injury [symbol] | | | 0.70 | | | 0.52 |
| Type of Injury [symbol] | | | 0.24 | | | 0.21 |
| GCS (total) (mean ± SD) | 5.3 ± 2.17 | 5.3 ± 2.5 | 0.95 | 4.5 ± 1.9 | 5.8 ± 2.3 | 0.11 |
| GCS-Motor | 2.9 ± 1.9 | 2.4 ± 2.2 | 0.54 | 2.28 ± 2.0 | 2.7 ± 2.1 | 0.57 |
| GCS-Eye (mean ± SD) | 1.5 ± 1.1 | 1.0 ± 0.0 | 0.14 | 1.4 ± 0.99 | 1.6 ± 1.3 | 0.68 |
| GCS-Verbal | 1.0 ± 0.75 | 1.1 ± 0.78 | 0.94 | 0.71 ± 0.48 | 1.13 ± 0.74 | 0.08 |
| GCS 3-4 | 15 (42.5%) | 5 (55.5%) | 0.80 | 9 (64.2%) | 6 (40%) | 0.62 |
| GCS 5-6 (mean ± SD) | 6 (14.1%) | 0 | | 1 (7.1%) | 1 (6.6%) | |
| GCS 7-8 | 14 (40%) | 4 (44.4%) | | 4 (28.5%) | 8 (53.3%) | |
| CT Findings** | | | | | | |
| Diffuse Axonal Injury | 5/20 | 0/7 | 0.57 | 2/7 | 2/11 | 0.62 |
| Mid Shift | 6/18 | 4/3 | 0.41 | 4/4 | 6/7 | 0.16 |
| Skull Fracture | 209/6 | 3/4 | 0.30 | 7/2 | 9/4 | 0.52 |
| Cerebral Edema | 6/20 | 0/7 | 0.24 | 3/6 | 2/11 | 0.49 |
| Contusion | 14/12 | 2/5 | 0.33 | 3/6 | 7/6 | 0.47 |
| Intracranial Hemorrage | 18/8 | 3/4 | 0.53 | 5/4 | 8/5 | 0.63 |
| Epidural Hemorrhage | 3/23 | 0/7 | 0.62 | 7/2 | 9/4 | 0.11 |
| Subdural Hemorrhage | 20/6 | 5/2 | 0.34 | 8/2 | 8/4 | 0.86 |
| Arachnoid Hemorrage | 22/4 | 5/2 | 0.37 | 6/3 | 8/5 | 0.16 |
| Marshall Score | | | | | | |
| I | 0 | 0 | | 0 | 0 | |
| II | 17 | 3 | | 4 | 6 | |
| III | 4 | 1 | 0.19 | 1 | 2 | 0.37 |
| IV | 3 | 0 | | 3 | 1 | |
| V | 2 | 3 | | 1 | 4 | |

Only, age and injury severity score (ISS) were significantly (p<0.05) associated with unfavorable outcome at 3 months, but not significant at 12 months. There was a significant difference in age and ISS between patients who died (n=21) and those who survived (n=23) at 3 months, with older age and higher ISS associated with unfavorable outcome. The cut points of ISS and age were determined at <75 and ≥49, respectively, the significant predictors to separate non-survivors from survivors at 3-month. Also, the cut points were calculated for Marshall score=4 and GCS=6 between non-survivors and survivors. These two variables were not statistically significantly different between the two cohorts.

Identified, Quantified Metabolites. 130 and 58 metabolites from different metabolite classes were identified and quantified using targeted DI/LC-MS/MS and untargeted ¹H-NMR, respectively, as outlined in Table 19 and Table 20.

Twenty-four of the 30 common metabolites measured by each technique had a similar trend of change, showing the accuracy of both techniques.

Metabolomics for the prognosis of 3 and 12 month outcomes of sTBI. The prediction models described illustrated that a serum metabolic biosignature can be used to prognosticate GOSE outcome at 3 and 12 months and the mortality outcome at 3 months.

Unsupervised PCA showed a relatively good grouping between cohorts with unfavorable and favorable outcome using all metabolites detected in serum samples collected on days 1 and 4. PCA revealed a high level of variability (R²X>0.5) of metabolic biosignature between two cohorts. Metabolic biosignatures obtained by DI/LC-MS/MS using samples on day 4 presented clearer groupings between unfavorable and favorable cohorts compared with ¹H-NMR and samples on day 1. PLS-DA-based analysis demonstrated a good predictive (Q²>0.5), highly significant (p<0.001) and highly sensitive and specific (>99%) prediction model to discriminate between patients with unfavorable vs favorable outcomes using a serum metabolic biosignature on day 4 obtained by DI/LC-MS/MS.

Table 22 shows the prediction models' characteristics show a higher predictability of metabolic profiles on day 4 than day 1 post-sTBI for 3 and 12 month GOSE and mortality at 3 month outcome. Additionally, the metabolic profiles obtained by DI/LC-MS/MS are more predictive than ¹H-NMR results. R², the goodness of fit of the model; Q², the goodness of prediction of the model; and AUC, area under the receiver operating curve of the model.

TABLE 22
Prediction Model Characteristics

| | Analytical | Sampling | | | | | | | # |
|---|---|---|---|---|---|---|---|---|---|
| Prognosis | Platforms | Time | R² | Q² | p value | Sensitivity | Specificity | AUC | Metabolites |
| Poor vs. | MS/MS | Day 1 | 0.60 | 0.40 | 0.0004 | 93 | 100 | 0.99 | 26 |
| Good | | Day 4 | 0.75 | 0.54 | 0.0003 | 100 | 100 | 1.00 | 24 |
| outcome | ¹H-NMR | Day 1 | 0.47 | 0.25 | 0.017 | 72 | 100 | 0.92 | 10 |
| 3-month | | Day 4 | 0.75 | 0.59 | 0.0001 | 100 | 96 | 1.00 | 9 |
| Poor vs. | MS/MS | Day 1 | 0.88 | 0.58 | 0.0002 | 100 | 100 | 0.99 | 21 |
| Good outcome | | Day 4 | 0.79 | 0.62 | 0.0004 | 100 | 100 | 0.98 | 29 |
| 12-month | ¹H-NMR | Day 1 | 0.64 | 0.46 | 0.003 | 76 | 91 | 0.91 | 12 |
| | | Day 4 | 0.7 | 0.41 | 0.044 | 100 | 100 | 1.00 | 9 |
| Mortality | MS/MS | Day 1 | 0.54 | 0.35 | 0.002 | 79 | 100 | 0.98 | 19 |
| outcome | | Day 4 | 0.76 | 0.50 | 0.0006 | 100 | 100 | 1.00 | 16 |
| | ¹H-NMR | Day 1 | 0.50 | 0.24 | 0.01 | 84 | 87 | 0.88 | 17 |
| | | Day 4 | 0.61 | 0.39 | 0.011 | 91 | 90 | 0.96 | 16 |

Nonetheless, day 1 metabolic biosignatures were also significant predictors for GOSE outcomes. The permutation analysis (200 times permuted, not shown) verified that the models are valid and unlikely to be overfit. Artificial neural network analysis (ANN), a machine learning-based analysis indicated the higher predictability (AUC>0.90) for the prognosis of GOSE outcome among patients with unfavorable outcome compared with patients with favorable outcome at 3 months and higher predictability (AUC>0.90) for the prognosis of GOSE outcome among patients with favorable outcome compared with patients with unfavorable outcome at 12-month. This is based primarily on DI/LC-MS/MS data on day 4.

Further analyses were performed to illustrate the relative correlation of the most differentiating metabolites between unfavorable and favorable outcomes for each prediction model-based list of metabolites including 9 to 26 metabolites for different models.

Table 23 shows relative concentration correlation of the metabolite alterations between the two cohorts with unfavorable and favorable GOSE outcome at 3 months on day 1 post-injury samples based on the DI/LC-MS/MS dataset. Fold change is also displayed for each metabolite.

TABLE 23
GOSE 3-month (Day 1) MS/MS
Fold Change is Increase or Decrease in Patients with Unfavourable Outcome

| | Mean (SD) of | Mean (SD) of | | Fold | Unfavorable/ |
|---|---|---|---|---|---|
| Name | Favorable outcome | Unfavorable Outcome | p-value | Change | Favorable |
| LYSOC17:0 | 0.530 (0.091) | 0.732 (0.241) | 0.0003 | 1.38 | UP |
| LYSOC16:0 | 38.088 (9.033) | 48.442 (14.023) | 0.0422 | 1.27 | Up |
| LYSOC18:0 | 8.556 (2.083) | 12.521 (4.367) | 0.0662 (W) | 1.32 | Up |
| C18 | 0.024 (0.005) | 0.032 (0.012) | 0.0662 (W) | 1.32 | Up |
| C18:2 | 0.032 (0.010) | 0.046 (0.023) | 0.0295 (W) | 1.45 | Up |
| Histidine | 96.527 (22.009) | 77.035 (18.290) | 0.009 | −1.25 | Down |
| Glutamine | 467.018 (72.297) | 364.854 (105.588) | 0.0092 | −1.28 | Down |
| Methionine | 18.537 (2.669) | 15.130 (5.773) | 0.0152 | −1.23 | Down |
| Phenylalanine | 77.266 (20.639) | 62.451 (16.749) | 0.0292 | −1.24 | Down |
| Glutamic acid | 65.982 (21.544) | 50.805 (17.553) | 0.0327 | −1.3 | Down |
| Tyrosine | 35.836 (7.295) | 29.787 (7.877) | 0.0433 | −1.2 | Down |
| Methionine-sulfoxide | 0.901 (0.302) | 0.605 (0.256) | 0.0065 (W) | −1.49 | Down |
| Isoleucine | 63.284 (16.785) | 47.666 (22.718) | 0.0180 (W) | −1.33 | Down |
| Asparagine | 30.707 (6.731) | 24.935 (9.329) | 0.0180 (W) | −1.23 | Down |
| Threonine | 72.222 (15.831) | 63.244 (37.829) | 0.0273 (W) | −1.14 | Down |
| Leucine | 130.653 (38.222) | 103.716 (55.160) | 0.0466 (W) | −1.26 | Down |
| PC322AA | 3.378 (1.151) | 2.607 (0.821) | 0.0466 (W) | −1.3 | Down |
| SM20:2 | 0.900 (0.454) | 0.611 (0.239) | 0.0479 (W) | −1.47 | Down |

Table 24 shows relative concentration correlation of the metabolite alterations between the two cohorts with unfavorable and favorable GOSE outcome at 3 months on day 4 post-injury samples based on the MS/MS dataset. Fold change is displayed for select metabolites.

TABLE 24
GOSE 3-month (Day 4) MS/MS

| | Mean (SD) of | Mean (SD) of | | Fold | Unfavorable/ |
|---|---|---|---|---|---|
| Name | Favorable | Unfavorable | p-value | Change | Favorable |
| Uric acid | 174.750 (59.974) | 102.108 (43.449) | 0.0009 | 1.71 | Down |
| Glutamine | 440.799 (90.105) | 354.923 (79.954) | 0.0169 | 1.24 | Down |
| Serine | 93.096 (19.529) | 74.917 (17.059) | 0.0181 | 1.24 | Down |
| SM 22:2 OH | 5.905 (0.508) | 5.177 (1.256) | 0.0296 | 1.14 | Down |
| Betaine | 33.416 (14.184) | 24.632 (8.770) | 0.0475 | 1.36 | Down |
| Glycine | 183.912 (42.079) | 155.385 (55.625) | 0.0481 (W) | 1.18 | Down |
| C14:1 | 0.096 (0.030) | 0.075 (0.043) | 0.0481 (W) | 1.27 | Down |
| C16:1 | 0.047 (0.027) | 0.031 (0.012) | 0.0481 (W) | 1.52 | Down |
| PC36:0 aa | 2.919 (0.693) | 3.979 (1.686) | 0.0197 | −1.36 | UP |

Table 25 shows relative concentration correlation of the metabolite alterations between the two cohorts with unfavorable and favorable GOSE outcome at 3 months on day 1 post-injury samples based on the NMR dataset. Fold change is shown for select metabolites.

TABLE 25
GOSE 3-month (Day 1) NMR

| | Mean (SD) of | Mean (SD) of | | Fold | Unfavorable/ |
|---|---|---|---|---|---|
| Name | Favorable | Unfavorable | p-value | Change | Favorable |
| Glycerol | 2.583 (1.228) | 5.102 (3.593) | 0.0115 (W) | −1.98 | Up |
| Lactate | 30.611 (8.837) | 47.671 (19.590) | 0.0138 (W) | −1.56 | Up |
| Serine | 1.624 (0.463) | 2.234 (1.027) | 0.0232 (W) | −1.38 | Up |
| Glycine | 1.903 (0.440) | 2.716 (1.376) | 0.0345 (W) | −1.43 | Up |
| Betaine | 0.815 (0.323) | 1.169 (0.619) | 0.0402 (W) | −1.43 | Up |
| Choline | 0.144 (0.034) | 0.190 (0.065) | 0.0402 (W) | −1.32 | Up |

Table 26 shows relative concentration correlation of the metabolite alterations between the two cohorts with unfavorable and favorable GOSE outcome at 3 months on day 4 post-injury samples based on the NMR dataset. Fold change shown for select metabolites.

TABLE 26
GOSE 3-month (Day 4) NMR

| | Mean (SD) of | Mean (SD) of | | Fold | Unfavorable/ |
|---|---|---|---|---|---|
| Name | Favorable | Unfavorable | p-value | Change | Favorable |
| Lactate | 19.621 (5.153) | 26.251 (3.743) | 0.0006 | −1.34 | Up |
| Valine | 4.350 (1.500) | 6.056 (1.080) | 0.0018 | −1.39 | Up |
| N-Acetylaspartate | 0.517 (0.211) | 0.754 (0.204) | 0.0093 | −1.46 | Up |
| Arginine | 1.967 (0.602) | 3.101 (1.176) | 0.0152 | −1.58 | Up |
| Lysine | 3.325 (1.027) | 4.583 (1.419) | 0.03 | −1.38 | Up |
| 2-Aminobutyrate | 1.283 (0.477) | 1.774 (0.554) | 0.035 | −1.38 | Up |
| Choline | 0.143 (0.048) | 0.195 (0.062) | 0.0406 | −1.37 | Up |
| Adipate | 0.133 (0.029) | 0.168 (0.065) | 0.0497 | −1.27 | Up |
| Tyrosine | 2.147 (0.789) | 3.113 (0.688) | 0.0030 (W) | −1.45 | Up |
| Gluconate | 2.038 (1.507) | 0.906 (0.585) | 0.0099 (W) | 2.25 | Down |
| Histidine | 1.161 (0.389) | 1.388 (0.235) | 0.0134 (W) | −1.2 | Up |
| Glutamate | 1.409 (0.501) | 2.475 (1.468) | 0.0237 (W) | −1.76 | Up |
| Urea | 0.434 (0.213) | 1.009 (0.864) | 0.0237 (W) | −2.33 | Up |
| Isoleucine | 1.637 (0.562) | 2.085 (0.602) | 0.0270 (W) | −1.27 | Up |
| Alanine | 3.529 (1.538) | 4.811 (1.147) | 0.0349 (W) | −1.36 | Up |
| Leucine | 2.788 (0.816) | 3.610 (1.135) | 0.0446 (W) | −1.29 | Up |

Table 27 shows relative concentration correlation of the metabolite alterations between the two cohorts with unfavorable and favorable GOSE outcome at 12 months on day 1 post-injury samples based on the MS/MS dataset. Fold change is shown for select metabolites.

TABLE 27
GOSE 12-month (Day 1) MS/MS

| | Mean (SD) of | Mean (SD) of | | Fold | Unfavorable/ |
|---|---|---|---|---|---|
| Name | Favorable | Unfavorable | p-value | Change | Favorable |
| Trans-Hydroxyproline | 5.250 (1.661) | 3.521 (0.957) | 0.0018 | −1.49 | Down |
| Methionine-sulfoxide | 0.814 (0.328) | 0.573 (0.193) | 0.0221 | −1.42 | Down |
| Acetyl-ornithine | 0.568 (0.269) | 0.383 (0.160) | 0.0315 | −1.48 | Down |
| Dimethylarginine A | 0.394 (0.140) | 0.292 (0.102) | 0.0318 | −1.35 | Down |
| Serine | 74.190 (17.235) | 60.344 (16.826) | 0.0374 | −1.23 | Down |
| Spermine | 0.203 (0.082) | 0.147 (0.030) | 0.0027 (W) | −1.38 | Down |
| PC360AE | 0.897 (0.263) | 0.708 (0.193) | 0.0470 (W) | −1.27 | Down |

Table 28 shows relative concentration correlation of the metabolite alterations between the two cohorts with unfavorable and favorable GOSE outcome at 12 months on day 4 post-injury samples based on the MS/MS dataset. Fold change is shown for select metabolites.

TABLE 28
GOSE 12-month (Day 4) MS/MS

| | Mean (SD) of | Mean (SD) of | | Fold | Unfavorable/ |
|---|---|---|---|---|---|
| Name | Favorable | Unfavorable | p-value | Change | Favorable |
| Tyrosine | 39.250 (7.928) | 46.480 (8.290) | 0.0362 | 1.18 | Up |
| Creatinine | 126.029 (56.501) | 171.078 (47.437) | 0.041 | 1.36 | Up |
| Betaine | 36.109 (14.187) | 25.021 (8.153) | 0.0238 | −1.44 | Down |
| C18 | 0.039 (0.017) | 0.024 (0.009) | 0.0055 (W) | −1.58 | Down |
| C142 | 0.039 (0.007) | 0.029 (0.017) | 0.0066 (W) | −1.34 | Down |
| Aspartic acid | 12.717 (7.980) | 7.108 (3.371) | 0.0457 (W) | −1.79 | Down |

Table 29 shows relative concentration correlation of the metabolite alterations between the two cohorts with unfavorable and favorable GOSE outcome at 12 months on day 1 post-injury samples based on the NMR dataset. Fold change is shown for select metabolites.

TABLE 29
GOSE 12-month (Day 1) NMR

| | Mean (SD) of | Mean (SD) of | | Fold | Unfavorable/ |
|---|---|---|---|---|---|
| Name | Favorable | Unfavorable | p-value | Change | Favorable |
| Alanine | 4.450 (1.081) | 5.999 (2.521) | 0.0483 | −1.35 | Up |
| 3-Hydroxyisovalerate | 0.048 (0.026) | 0.029 (0.012) | 0.0328 (W) | −1.68 | Down |
| Ornithine | 0.719 (0.276) | 1.183 (0.799) | 0.0367 (W) | 1.65 | UP |

Table 30 shows relative concentration correlation of the metabolite alterations between the two cohorts with unfavorable and favorable GOSE outcome at 12 months on day 4 post-injury samples based on the NMR dataset. Fold change shown for select metabolites.

TABLE 30
GOSE 12-month (Day 4) NMR

| | Mean (SD) of | Mean (SD) of | | Fold | Unfavorable/ |
|---|---|---|---|---|---|
| Name | Favorable | Unfavorable | p-value | Change | Favorable |
| Tyrosine | 2.299 (0.680) | 3.137 (0.850) | 0.0156 | 1.36 | Up |
| Valine | 4.464 (1.181) | 5.851 (1.445) | 0.0193 | 1.31 | Up |
| Alanine | 3.903 (1.398) | 5.320 (1.661) | 0.0374 | 1.36 | Up |
| Ornithine | 1.149 (0.495) | 1.588 (0.478) | 0.0443 | 1.38 | Up |
| Dimethyl sulfone | 0.059 (0.026) | 0.571 (1.041) | 0.0178 (W) | 9.7 | Up |

Table 31 shows relative concentration correlation of the metabolite alterations between non-survivor and survivor cohorts at 3 months on day 1 post-injury based on the DI/LC-MS/MS data.

TABLE 31
Mortality outcome (Day 1) MS/MS

| | | | | Fold | |
|---|---|---|---|---|---|
| Name | Mean (SD) of Alive | Mean (SD) of Died | p-value | Change | Alive/Died |
| Isoleucine | 59.398 (23.850) | 41.511 (16.616) | 0.0066 | 1.43 | Up |
| Glutamine | 426.519 (101.926) | 341.101 (96.682) | 0.0068 | 1.25 | Up |
| Histidine | 87.192 (22.533) | 74.265 (15.755) | 0.0345 | 1.17 | Up |
| C3:1 | 0.042 (0.013) | 0.050 (0.011) | 0.0532 | −1.17 | Down |
| Valine | 156.222 (50.515) | 121.700 (37.588) | 0.0177 (W) | 1.28 | Up |
| Leucine | 127.249 (60.615) | 89.486 (34.529) | 0.0261 (W) | 1.42 | Up |
| Citrulline | 20.039 (8.801) | 15.305 (7.297) | 0.0278 (W) | 1.31 | Up |
| PC38:0AA | 1.164 (0.351) | 1.290 (0.285) | 0.0364 (W) | −1.11 | Down |

Table 32 shows relative concentration correlation of the metabolite alterations between non-survivor and survivor cohorts based on day 4 post-injury based on the DI/LC-MS/MS data.

TABLE 32
Mortality outcome (Day 4) MS/MS

| | | | | Fold | |
|---|---|---|---|---|---|
| Name | Mean (SD) of Alive | Mean (SD) of Died | p-value | Change | Died/Alive |
| Taurine | 50.190 (16.576) | 32.495 (15.675) | 0.0062 | 1.54 | Down |
| Glutamine | 410.051 (84.099) | 324.887 (74.218) | 0.0076 | 1.26 | Down |
| LYSOC26:0 | 0.511 (0.181) | 0.334 (0.152) | 0.0088 | 1.53 | Down |
| C6 | 0.086 (0.046) | 0.052 (0.016) | 0.0009 (W) | 1.66 | Down |
| C12:1 | 0.223 (0.070) | 0.171 (0.058) | 0.0227 (W) | 1.31 | Down |
| Creatinine | 141.578 (62.375) | 97.911 (65.780) | 0.0317 (W) | 1.45 | Down |
| C14:1 | 0.086 (0.032) | 0.071 (0.052) | 0.0353 (W) | 1.21 | Down |
| C8 | 0.239 (0.224) | 0.141 (0.039) | 0.0392 (W) | 1.7 | Down |
| Glycine | 176.007 (54.428) | 141.751 (46.064) | 0.0435 (W) | 1.24 | Down |
| C10 | 0.169 (0.126) | 0.114 (0.036) | 0.0435 (W) | 1.48 | Down |

Table 33 shows relative concentration correlation of metabolite alterations between non-survivor and survivor cohorts at 3-mos on day 1 post-injury based on NMR data.

TABLE 33
Mortality outcome (Day 1) NMR

| | | | | Fold | |
|---|---|---|---|---|---|
| Name | Mean (SD) of Alive | Mean (SD) of Died | p-value | Change | Died/Alive |
| Glucose | 261.897 (75.982) | 322.188 (92.781) | 0.0226 | −1.23 | Up |
| Fumarate | 0.032 (0.014) | 0.044 (0.018) | 0.0255 | −1.35 | Up |
| Isoleucine | 1.303 (0.538) | 0.991 (0.358) | 0.0177 (W) | 1.32 | Down |
| Betaine | 0.896 (0.309) | 1.315 (0.733) | 0.0230 (W) | −1.47 | Up |
| Leucine | 2.468 (0.983) | 1.927 (0.753) | 0.0401 (W) | 1.28 | Down |
| Citrate | 4.176 (1.657) | 5.500 (2.384) | 0.0477 (W) | −1.32 | Up |

Table 34 shows relative concentration correlation of the metabolite alterations between non-survivor and survivor cohorts at 3-mos on day 4 post-injury based on NMR data.

TABLE 34
Mortality outcome (Day 4) NMR

| | | | | Fold | |
|---|---|---|---|---|---|
| Name | Mean (SD) of Alive | Mean (SD) of Died | p-value | Change | Alive/Died |
| Valine | 5.110 (1.184) | 6.349 (1.320) | 0.0121 | −1.24 | Up |
| Lysine | 3.668 (1.206) | 4.961 (1.535) | 0.0154 | −1.35 | Up |
| Isobutyrate | 0.183 (0.056) | 0.238 (0.068) | 0.0223 | −1.3 | Up |
| 2-Aminobutyrate | 1.478 (0.426) | 1.918 (0.632) | 0.0301 | −1.3 | Up |
| Gluconate | 1.458 (1.041) | 0.667 (0.376) | 0.0016 (W) | 2.19 | Down |
| Betaine | 1.125 (0.387) | 0.830 (0.359) | 0.0346 (W) | 1.35 | Down |
| 3-Hydroxyisovalerate | 0.096 (0.216) | 0.067 (0.029) | 0.0387 (W) | 1.44 | Down |

A predictive metabolic biosignature to predict GOSE outcome at 3-months was characterized by an increased in lysoPCs, propionic acid, stearic acid, oleic acid, linoleic acid, myristic acid, choline, acylcarnitine, glycerol, glucose, lactate, pyruvate, tryptophan, homocysteine, and ketone bodies (2-hydroxybutyric acid, acetoacetate, and acetone) on the 1st day post-injury yielding an unfavorable outcome. Interestingly, a predictive metabolic biosignature on day 4 showed increased glutamate (excitotoxicity), phenylalanine, tyrosine, kynurenine, NAA, aspartate, and branched chained amino acids (valine, leucine, and isoleucine) in those with an unfavorable outcome, while these metabolites were decreased on day 1 post-injury. For prognosis of GOSE at 12-months, patients with unfavorable outcome were characterized by increased lysoPCs, short chain ACs, palmitic acid, oleic acid, linoleic acid, lactate, gluconate, branched chain amino acids, carnitine, glycerol, alanine, and a decrease in spermine, methionine-sulfoxide, glutamate, ketone bodies, hydroxyisovalerate compounds, and dimethylamine on day 1 post-injury. 12 month unfavorable outcome was associated with increased lysoPCs, tryptophan, caproic acid, lauric, and lauroleic (9-dodecanoid) acids, oleic acid, tyrosine, branched chain amino acids and ornithine on day 4 post-injury. To predict 3 month mortality, metabolomic analysis showed increased glucose, PCs, long chain acylcarnitines (oleic acid, linoleic acid, palmitoleic acid, myristolinoleic acid, lauroleic acid, capric acid, and myristoleic acid), TCA cycle metabolites, tryptophan, tyrosine, and ketone bodies in deceased patients on day 1 and 4 post-injury. Deceased patients showed decreased short chain acylcarnitines, glutamine, and betaine on day 4. Univariate T-test analysis showed remarkable similarities to PLS-based prediction models to identify predictive biomarkers.

A brief overview highlights that unfavorable outcome was associated with increased metabolites related to lipids and anaerobic metabolism and decreased metabolites related to serotonergic, polyamine metabolism and NMDA receptor integrity in day 1 post-injury. Increased metabolites related to neuroinflammation, excitotoxicity and brain injury specific biomarkers were found on day 4 post-injury. Also, notable was an association of increased metabolites related to acylcarnitines metabolism and energy metabolism with mortality.

Clinical variables for the prognosis of GOSE outcome at 3 month, 12 months, and mortality. It was investigated whether clinical variables could predict the outcome of sTBI at 3 and 12 months post sTBI. Statistically inspired modification of partial least square (SIMPLS) analysis revealed the most differentiating clinical variables for predicting outcomes at 3 months (age, ISS, Marshall score and hypoxemia) and 12 months (age, GCS, hypoxemia, and loss of consciousness). However, these clinical variables had low prediction capacity (Q²<0.16) and less sensitivity (66%) and specificity (86%) compared to metabolites (Table S6). SIMPLS analysis of clinical data revealed that age and severity of illness score (ISS) are useful predictors (Q²=0.37, AUC=0.86) to prognosticate mortality. However, these clinical variables lack significant sensitivity and specificity (66%-83%) compared to metabolomics data.

The prognosis of the unfavorable GOSE outcome cohort showed different predictabilities at 3 and 12 months. ANN revealed that the prediction of unfavorable GOSE outcome was more accurate (AUC>0.97) than the prediction of favorable GOSE outcome according to training and validation sets (Table S4). A higher level of predictability for the prognosis of unfavorable GOSE outcome was observed using day 4 samples and the DI/LC-MS/MS dataset compared to the day 1 dataset. Of note, the DI/LC-MS/MS dataset was better than the ¹H-NMR dataset in predicting unfavorable GOSE outcome at 3 months. While the prognosis of favorable GOSE outcome was more predictive than the prognosis of unfavorable outcome at 12 months using both training and validation sets in ANN analysis of 1H-NMR dataset. Further analysis showed that the prognosis of GOSE outcome at 12 months overall was slightly less predictive than the prognosis of GOSE outcome at 3 months. Overall, the prognosis of 12 month GOSE outcome was less predictable than the prognosis of 3 month GOSE outcome.

ANN analysis indicates the prognosis of unfavorable outcome is more predictive than the prognosis of favorable GOSE outcome at 3 months. In addition, there is a higher predictability using day 4 data compared to day 1 data according to both training and validation sets. Further, ANN analysis indicates the prognosis of favorable GOSE outcome is more predictive than the prognosis of unfavorable outcome at 12 months as well as higher predictability of day 4 samples post sTBI compared to day 1 samples post sTBI.

SIMPLS analysis was used for the prediction of GOSE outcome using the most differentiating metabolites (VIP>1) (metabolomics) and a combination of the most differentiating metabolites (with VIP>1) and the most differentiating clinical variables (VIP>1). Only prediction models where the combined use of clinical variables and metabolomics could improve the predictability compared to metabolomics only prediction models; notably the 12 month prediction models were not improved with the clinical variables, and therefore are not shown here.

FIG. 9A and FIG. 9B show predictor screening analysis, illustrating the importance of clinical variables in the prediction models for the prognosis of GOSE outcome based on Day 1 serum samples (FIG. 9A) and Day 4 (FIG. 9B) serum samples at 3 months and 12 months using DI/LC-MS/MS data. The figures present the ranking of metabolites and clinical variables in each prediction model.

Identified, quantified metabolites using DI/LC-MS/MS and ¹H-NMR. 130 and 58 metabolites were quantified using targeted DI/LC-MS/MS and untargeted ¹H-NMR, respectively (Table 19 and Table 20). The quantified metabolites by the DI/LC-MS/MS platform included 75 lipids (glycerophospholipids, acylcarnitines, sphingomyelins), 22 amino acids, 23 biogenic amines, 17 organic acids, and several compounds from different metabolite classes. In addition, the quantified metabolites by 1H-NMR included 22 amino acids, 20 organic acids, 4 sugars and 12 biogenic acids for a total of 58 metabolites. Though there are several common metabolites between DI/LC-MS/MS and ¹H-NMR methods, the approaches to identify and quantify metabolites were completely different between the techniques. Both techniques were quantitative analyses in this study, but the quantification of metabolites was based on the ion intensities of metabolite fragmentations and the physical-chemistry of the hydrogen atom (proton ¹H) in intact metabolites for DI/LC-MS/MS and ¹H-NMR, respectively. Further analysis showed that 80% (24 out of 30) of the overlapping metabolites followed a similar trend of change that illustrates the accuracy of both techniques.

### Patients' Demographics, Clinical Information and CT Findings Between Non-Survivors and Survivors' Cohorts.

There was a significant difference in age and ISS between patients who died (n=21) and those who survived (n=23) at 3 months, with older age and higher ISS associated with unfavorable outcome.

Characterization of metabolite biosignature for the prognosis of GOSE outcome. A large number of metabolites contributed to the highly predictive (Q²>0.5) and significant separation (AUC>0.99) between cohorts with unfavorable and favorable GOSE outcomes at 3 and 12 months as well as non-survivors vs. survivors at 3 months. Nonetheless, further analyses showed that one may be able to decrease the number of metabolites and still build reasonable predictive (Q²>0.4) and accurate (AUC>0.90) models as shown in tables S4-S6. As the metabolites used in the models are decreased, there is an associated lower sensitivity (<80), specificity (<80), and AUC (<0.75) (data not shown). The following metabolic biosignature characterizations are based on the best prediction models.

3 month prognosis. Unfavorable outcome was characterized by an increase in lysophosphatidylcholine (lysoPCs), propionic acid, stearic acid, oleic acid, linoleic acid, myristic acid, choline, glycerol, glucose, lactate, pyruvate, tryptophan, homocysteine, and ketone bodies (2-hydroxybutyric acid, acetoacetate, and acetone) on the 1st day post-injury, while glutamate, phenylalanine, tyrosine, kynurenine, NAA, aspartate, and branched chained amino acids (valine, leucine, and isoleucine) increased from the 1st day to the 4th day post-injury.

12 month prognosis. Unfavorable outcome was characterized by an increase in lysophosphatidylcholines (lysoPCs), short chain acylcarnitines (ACs), palmitic acid, oleic acid, linoleic acid, lactate, gluconate, branched chain amino acids, carnitine, glycerol and alanine and a decrease in spermine, methionine-sulfoxide, glutamate, ketone bodies, hydroxyisovalerate compounds, and dimethylamine on day 1 after injury. On day 4 after injury there was an increase in lysoPCs, tryptophan, caproic acid, lauric acid, lauroleic acids, oleic acid, tyrosine, branched chain amino acids, and ornithine and a decrease in spermine, spermidine, PCs, most medium and long chain ACs and serotonin in patients with unfavorable outcome vs. the patients with favorable GOSE outcome at 12 months.

Prognosis of Mortality. Non-survivors were characterized by an increase in glucose, PCs, long chain acylcarnitines (oleic acid, linoleic acid, palmitoleic acid, myristolinoleic acid, lauroleic acid, capric acid, and myristoleic acid), TCA cycle metabolites, tryptophan, tyrosine, and ketone bodies on days 1 and 4 post-injury. There was a decrease in short chain acylcarnitines, glutamine and betaine that were correlated with a non-survival outcome on day 4 post injury.

The combination of metabolomics and clinical variables for the predicting GOSE outcome at 3 and 12 months post-injury. SIMPLS analysis demonstrated that clinical variables could moderately improve the performance of metabolomics-based prediction models to prognosticate only GOSE outcome at 3 months and mortality. For the prognosis of GOSE outcome at 12-month, clinical variables were found to minimally improve the metabolomics model (data not shown). However, age was an important clinical predictor of outcome among clinical variables, with a high level of contribution to prediction models, particularly for mortality. Consequently, Marshall score (3 months outcome) and GCS (12 months outcome) remain important clinical variable (Table S8). Although SIMPLS and PLS-DA use different algorithms, the two approaches showed overall similar predictabilities when metabolites were used to prognosticate sTBI outcomes, with only slight differences. Importantly, permutation tests (not shown) verified the predictabilities of metabolite-based prediction models and was used to help prevent overfitting of the data.

### Discussion

The current findings show that metabolite alterations on days 1 and 4 post-sTBI were highly-predictive and well-correlated with GOSE unfavorable and favorable outcomes at 3 and 12 months and importantly, may also be used as a promising prognostic tool to predict the worst GOSE outcome, i.e., death. The metabolic biosignatures on day 4 post-injury were more predictive and significant to prognosticate 3 and 12 month outcomes. From a total of 160 metabolites, multivariate analysis revealed that several metabolites contributed to the separation of groups with unfavorable versus favorable outcome, implying fundamental metabolic alterations with sTBI that allows one to predict outcome with good sensitivity, specificity, and AUC. The higher predictability of serum metabolic biosignatures on day 4 for the prognosis of outcomes may reflect the contribution of secondary brain injury in addition to primary brain injury (reflected by day 1 metabolites) that correlates with outcome. A remarkable similarity was found for the trends in changes in metabolites measured by two distinct methodologies, showing a high level of accuracy of quantification using two different analytical platforms. The current study demonstrated that subtle changes in the metabolic profiles correlate with known and unknown pathophysiological pathways that can be applied to predict 3 and 12 month outcomes.

Metabolomics appeared to be superior to patients' demographics, clinical features, and CT findings in predicting GOSE outcome at 3 and 12 months post injury. Notably, the combination of metabolomics with clinical and CT variables enhanced the metabolomics prognostication of sTBI outcome in the early days post-injury, though clinical and CT data only improved the metabolomics prediction models for the prognosis of GOSE outcome at 3 months but not 12 months. Addition of age, GCS, hypoxemia, injury severity score, and Marshall score apparently enhanced the performance of metabolomics-based prediction of outcome. These results were similar to the IMPACT and CRASH studies²³ in their use of age, GCS motor, pupillary reactivity, CT classification, EDH (epidural hematoma), tSAH (subarachnoid hemorrhage), hypoxia, and hypotension²³ and identified age, GCS motor, pupillary reactivity, hypoxia, hypotension and CT classification as the most important predictors of outcomes using multivariate analysis (AUC 0.83-0.89) that is also similar to the European Brain Injury Consortium Core Data (EBIC) and Traumatic Coma Data Bank (TCDB) studies.²⁴ It was shown that age and ISS are the most differentiating prognostic variables for mortality, while the IMPACT prediction model revealed age, GCS motor score, pupillary reactivity, hypoxia, hypotension, basal cisterns narrowing, midline shift and tSAH as the most predictive variables for 14 day mortality.²⁵ Using a multimodal approach, physiological (ICP, MAP, CPP and pbtO2) and biochemical (pyruvate, lactate, glycine, glutamate, and glucose) parameters could predict sTBI outcome with approximately 90% accuracy.²⁶ This study also demonstrates the importance of multivariate predictive and machine learning based-models versus simplified methods to determine predictive metabolites. A Bayesian networks approach previously showed an improvement in prediction models using variables that were not predictive in simplified models.²⁷

The current study suggests that, as previously described, increased lysoPCs in patients with unfavorable outcome may be correlated with microvascular barrier disruption, promotion of oligodendrocyte demyelination and pericyte loss and with induced inflammation.²⁹ Increased stearic acid (C18) and its derivatives (stearic acid, oleic acid, linoleic acid) and lysoPCs in those with unfavorable outcome may correlate with docosahexaenoic acid (DHA) metabolism, a highly enriched brain lipid.³⁰ Increased CSF levels of lysoPCs and PCs were previously observed in non-survivors and survivors³¹ respectively, and in mild TBI patients compared to non-concussed controls.³² Within one day post-sTBI, increased energy-related metabolites (lactate, glucose, and TCA cycle compounds) have been observed in patients with unfavorable outcomes. The lactate/pyruvate ratio is well-recognized as a predictor for the prognosis of brain injuries such as apoptosis, cerebral anoxia, and anaerobic metabolism.^{33,34} There was a correlation between elevated lactate with unfavorable outcomes in TBI, in association with reduced cerebral blood flow (CBF), elevated ICP, and ischemia.^{33,34} In this study, increased tryptophan, kynurenine, tyrosine, phenylalanine, and glutamate on day 4 post-injury may intriguingly imply the correlation of excessive excitotoxicity mechanisms³⁵ and aromatic amino acid metabolism³⁶ with unfavorable outcome. Increased quinolinic acid, the final product of the tryptophan-kynurenine pathway, has been associated with the inflammatory response due to infiltration of macrophages and activation of microglia in the CNS³⁷ and with unfavorable outcome and mortality in sTBI, indicating the possibility of elevated macrophage-derived (or microglia-derived) excitotoxins in the contribution of secondary injury to poor outcome.^{37,38} In addition, day 4 increased NAA and phenylalanine, two well-known neurotransmitters in patients with unfavorable outcomes, may be associated with alteration of osmolality and catecholaminergic mechanism of injury.³⁹ The current data also showed the association of day 1 hyperglycemia and increased lactate with poor outcome. Hyperglycemia and hyperlactatemia have been previously shown to be potential predictors for the prognosis of unfavorable TBI outcome.⁴⁰⁻⁴²

Current findings provide novel evidence of targeted metabolomic profiling for the prognosis of short and long-term GOSE outcome using serum samples at days 1 and 4 post-injury. A combination of amino acids, organic acids, fatty acids, clinical and CT findings as variables were defined to prognosticate GOSE outcome of sTBI among adult patients.

Limitations of the current study include a relatively small sample size, not all patients had GOSE outcomes measured (lost to follow-up), and there was a skewed cohort towards males (not uncommon in TBI studies), thus a larger and gender-balanced cohort could be used to further affirm these findings. It is controversial what role blood and blood product transfusion in trauma care plays in metabolites found in serum. In this study 15 patients had blood or blood product transfusion between 1 to 4 units. The transfusion was performed for 8 out of 15 patients after day 1 post injury and for 2 patients after day 4 post injury (thus not affecting measured metabolites), however the impact on serum metabolites is uncertain for those samples collected after transfusion. Further analysis on patients' demographics and clinical symptoms on admission date showed a random effect of those patients lost to follow up (i.e. there was no systematic loss to follow-up noted). Despite these limitations, this study shows great promise in using metabolomics to evaluate sTBI, particularly for prognostic assessment.

Metabolic profiling of sTBI patient samples beyond the first 4 days may potentially enhance the predictability of metabolomics to prognosticate outcome and may provide more definitive information about molecular changes post sTBI, especially in those who have a favorable outcome of sTBI. Also, applying an untargeted mass spectrometry approach may help identify more known and unknown metabolites that may be correlated with sTBI prognosis and help to more clearly define the mechanisms of injury in sTBI (for both primary and secondary injury).

In this study the prognostication models showed highly predictive and significant separation between sTBI patients with unfavorable and favorable outcomes using serum metabolomics with remarkable similarities between two different metabolomics analytical platforms while the patients' demographics and clinical variables were not strong independent predictors of GOSE outcome. Importantly, the information derived from metabolomics and prediction models may be used to stratify patients with sTBI that can be applied in future clinical trials, especially therapeutic trials as a means of prognostic enrichment. Targeted DI/LC-MS/MS (including multiple lipid metabolites) appears to be superior to ¹H-NMR to predict sTBI outcome and this information may be useful for future studies.

In summary, the best prognostic metabolomics models for unfavorable 3 month and 12 month GOSE outcomes include increased glycolytic metabolites, hyperglycemia, and lactate on day 1, increased aromatic amino acids (tryptophan, tyrosine, and phenylalanine) on day 4, metabolites involved in excitotoxicity (increased glutamate), increased neuroinflammation metabolites (increased lysoPCs and kynurenine) on both days 1 and 4, increased neurobiomarkers (increased NAA and tyrosine), decreased ketone bodies, decreased urea cycle metabolites and degradation of branched chain amino acids (BCAA) on day 4.

### Example 1B

### Metabolomic Profiles in Serum: Metabolite Lists, Sensitivity, Specificity, and Modeling for Predicting Global Functional Neurological Outcome at 3 and 12 Months and Death at 3 Months Following Severe Traumatic Brain Injury

The information from Example 1 and Example 1A was further analyzed in the context of most predictive metabolites depending upon stated conditions of: i) whether the blood sample was taken from Day 1 vs Day 4; ii) whether 3-month vs 12-month GOSE outcome was assessed; iii) whether and whether MS/MS versus NMR analysis was used; iv) whether mortality outcome was assessed. Larger metabolite groups were compared with smaller metabolite subsets to determine an optimal test in which sensitivity and sensitivity were adequately high relative to accurately predict outcome based on a minimum set of metabolites.

In this example, the metabolites of interest are assessed as biomarkers, such that the number of metabolomic variables are minimized and optimized to develop significant predictive models while using the fewest biomarkers to accurately represent the parameter of poor vs. good outcome, GOSE at 3-month or 12-month; or mortality. Prediction models were assessed with decreasing number of metabolites using PLS-DA modeling.

The following tables show change relative to control for specific metabolites under the conditions as indicted.

Table 35A indicates MS/MS profile of a Day 1 serum sample, GOSE 3-month for 26 metabolites.

TABLE 35A
MS/MS Day 1, GOSE 3-month (26 metabolites)

| | | Fold | Poor/Good |
|---|---|---|---|
| Metabolites | p-value | Change | outcome |
| Lyso PC 17:0 | 0.003 | 1.38 | Up |
| Lyso PC 18:0 | 0.0066 | 1.32 | Up |
| C3:1 | 0.437 | 1.08 | Up |
| Lyso PC 16:0 | 0.0422 | 1.27 | Up |
| Lyso PC 18:1 | 0.115 | 1.27 | Up |
| C18:2 | 0.0295 | 1.45 | Up |
| C14 | 0.0662 | 1.32 | Up |
| C18 | 0.0066 | 1.32 | Up |
| C18:1 | 0.0062 | 1.34 | Up |
| C16 | 0.0757 | 1.26 | Up |
| C14:2 | 0.204 | 1.45 | Up |
| Tyrosine | 0.0433 | −1.2 | Down |
| Asparagine | 0.018 | −1.23 | Down |
| PC ae 36:0 | 0.0606 | −1.26 | Down |
| C16:2 | 0.3358 | 1.3 | Up |
| Phenylalanine | 0.0292 | −1.24 | Down |
| C16:1 | 0.373 | 1.22 | Up |
| Glutamine | 0.0092 | −1.28 | Down |
| SM 20:2 | 0.479 | −1.47 | Down |
| PC aa 32:2 | 0.0466 | −1.3 | Down |
| Isoleucine | 0.018 | −1.23 | Down |
| Citrulline | 0.0538 | −1.38 | Down |
| Histidine | 0.009 | −1.25 | Down |
| Glutamate | 0.0327 | −1.3 | Down |
| Methionine-Sulfoxide | 0.0066 | −1.49 | Down |
| Asymmetric | 0.0506 | −1.38 | Down |
| dimethylargine | | | |

Table 35B indicates MS/MS profile of a Day 1 serum sample, GOSE 3-month for 13 metabolites.

TABLE 35B
MS/MS Day 1, GOSE 3-month (13 metabolites)

| | | Fold | Poor/Good |
|---|---|---|---|
| Metabolites | p-value | Change | outcome |
| LYSOC17:0 | 0.003 | 1.38 | Up |
| LYSOC18:0 | 0.0066 | 1.32 | Up |
| LYSOC16:0 | 0.0422 | 1.27 | Up |
| C18:2 | 0.0295 | 1.45 | Up |
| C14 | 0.0662 | 1.32 | Up |
| C18:1 | 0.0295 | 1.45 | Up |
| C18 | 0.0066 | 1.32 | Up |
| C16 | 0.0757 | 1.26 | Up |
| Glutamine | 0.0092 | −1.28 | Down |
| Histidine | 0.009 | −1.25 | Down |
| SM 20:2 | 0.479 | −1.47 | Down |
| Methionine-sulfoxide | 0.0066 | −1.49 | Down |
| Asymmetric dimethylarginine | 0.0506 | −1.38 | Down |

Regarding Tables 35A and 35B, a Multivariate Data Analysis (OPLS-DA/PLS-DA) for Day 1 serum with MS/MS analysis (Poor vs. Good outcome 3-months) is represented in Table 35C, showing (*) optimized prediction using 26 and 13 metabolites, versus 48, 40, 32, and 21 metabolites.

TABLE 35C
Multivariate Data Analysis (OPLS-DA/PLS-DA)-Day 1 MS/MS
(Poor vs. Good outcome GOSE 3-months)

| Analytical | Sampling | | | p | | | | | | |
|---|---|---|---|---|---|---|---|---|---|---|
| Platforms | Time | R2 | Q2 | value | Sensitivity | Specificity | AUROC | VIP > | Metabolites | Patients |
| MS/MS | Day 1 | 0.739 | 0.393 | 0.0015 | 100 | 100 | 0.99 | 1 | 48 | Poor |
| | Day 1 | 0.593 | 0.377 | 0.001 | 78 | 100 | 0.94 | 1.1 | 40 | outcome |
| | Day 1 | 0.593 | 0.392 | 0.0006 | 91 | 100 | 0.96 | 1.2 | 32 | N = 35 |
| | Day 1* | 0.596 | 0.398 | 0.0004 | 93 | 100 | 0.99 | 1.3 | 26* | Good |
| | Day 1* | 0.558 | 0.342 | 0.002 | 80 | 95 | 0.92 | 1.4 | 21* | outcome |
| | Day 1* | 0.503 | 0.354 | 0.0017 | 65 | 100 | 0.85 | 1.5 | 13* | N = 9 |

Table 36A indicates MS/MS analysis of metabolites in a Day 4 serum sample, predictive of GOSE 3-month for 15 metabolites.

TABLE 36A
MS/MS Day 4, GOSE 3-month (15 metabolites)

| | | Fold | Poor/Good |
|---|---|---|---|
| Metabolites | p-value | Change | outcome |
| C3OH | 0.0029 | 1.33 | Up |
| Glutamic acid | 0.0164 | 1.31 | Up |
| LYSOC18:0 | 0.0155 | 1.49 | Up |
| Ornithine | 0.044 | 1.31 | Up |
| PC aa3 6:0 | 0.0048 | 1.52 | Up |
| C18:2 | 0.119 | 1.26 | Up |
| alpha-Aminoadipic acid | 0.0339 | 1.33 | Up |
| Indole acetic acid | 0.172 | 1.93 | Up |
| C3:1 | 0.061 | 1.24 | Up |
| PC aa 40:2 | 0.963 | −1.01 | Down |
| C16:1 | 0.249 | −1.37 | Down |
| Serine | 0.264 | −1.1 | Down |
| Glutamine | 0.294 | −1.1 | Down |
| β-Hydroxybutyric acid | 0.0538 | −5.24 | Down |
| Uric acid | 0.0422 | −1.48 | Down |

Table 36B indicates MS/MS analysis of a Day 4 serum sample, for prediction of GOSE 3-month for 11 metabolites.

TABLE 36B
MS/MS Day 4, GOSE 3-month (11 metabolites)

| | | Fold | Poor/Good |
|---|---|---|---|
| Metabolites | p-value | Change | outcome |
| C3OH | 0.0029 | 1.33 | Up |
| Glutamic acid | 0.0164 | 1.31 | Up |
| LYSOC18:0 | 0.0155 | 1.49 | Up |
| Ornithine | 0.044 | 1.31 | Up |
| PC36:0AA | 0.0048 | 1.52 | Up |
| C18:2 | 0.119 | 1.26 | Up |
| C3:1 | 0.061 | 1.24 | Up |
| C16:1 | 0.249 | −1.37 | Down |
| Glutamine | 0.294 | −1.1 | Down |
| beta-Hydroxybutyric acid | 0.0538 | −5.24 | Down |
| Uric acid | 0.0422 | −1.48 | Down |

Regarding Tables 36A and 36B, a Multivariate Data Analysis (OPLS-DA/PLS-DA) for Day 4 MS/MS analysis (Poor vs. Good outcome 3-months) GOSE 3-month is represented in Table 36C, showing (*) optimized prediction using 15 and 11 metabolites, versus 54, 39, 28, or 24 metabolites.

TABLE 36C
Multivariate Data Analysis (OPLS-DA/PLS-DA)
Day 4 MS/MS analysis
(Poor vs. Good outcome 3-months)

| Analytical | Sampling | | | | | | | | | |
|---|---|---|---|---|---|---|---|---|---|---|
| Platforms | Time | R2 | Q2 | p value | Sensitivity | Specificity | AUROC | VIP > | Metabolites | Patients |
| MS/MS | Day 4 | 0.81 | 0.61 | 5.4 × 10⁻⁵ | 100 | 100 | 0.99 | 1 | 54 | Poor |
| | Day 4 | 0.82 | 0.662 | 1.2 × 10⁻⁵ | 100 | 100 | 0.99 | 1.1 | 39 | outcome |
| | Day 4 | 0.754 | 0.568 | 0.0001 | 100 | 100 | 1 | 1.2 | 28 | n = 23 |
| | Day 4 | 0.753 | 0.543 | 0.0003 | 100 | 100 | 1 | 1.3 | 24 | Good |
| | Day 4* | 0.574 | 0.464 | 0.00016 | 100 | 100 | 1 | 1.4 | 15* | outcome |
| | Day 4* | 0.543 | 0.462 | 0.00017 | 82 | 100 | 0.95 | 1.5 | 11* | n = 8 |

Table 37A indicates MS/MS analysis of a Day 1 serum sample, for prediction of GOSE outcome at 12-month for 21 metabolites.

TABLE 37A
MS/MS Day 1, GOSE 12-month (21 metabolites)

| | | Fold | Poor/Good |
|---|---|---|---|
| Metabolites | p-value | Change | outcome |
| C5OH | 0.6041 | −1.04 | Down |
| Homocysteine | 0.344 | −1.09 | Up |
| C3 | 0.085 | 1.27 | Up |
| C0 | 0.504 | 1.31 | Up |
| C4 | 0.2898 | 1.26 | Up |
| Ornithine | 0.0581 | 1.36 | Up |
| LYSOC14:0 | 0.088 | 1.19 | Up |
| SM 16:1 OH | 0.4628 | 1.05 | Up |
| LYSOC20:3 | 0.234 | 1.13 | Up |
| LYSOC28:1 | 0.1261 | 1.26 | Up |
| C10:2 | 0.3536 | −1.14 | Down |
| Acetyl-ornithine | 0.0315 | −1.48 | Down |
| C9 | 0.133 | −1.21 | Down |
| Adimethylarginine | 0.0318 | −1.35 | Down |
| Methionine-sulfoxide | 0.0221 | −1.42 | Down |
| Spermine | 0.0027 | −1.38 | Down |
| PC ae 36:0 | 0.047 | −1.27 | Down |
| Citrulline | 0.2172 (W) | −1.17 | Down |
| Serotonin | 0.084 | −1.27 | Down |
| Serine | 0.0347 | −1.23 | Down |
| trans-Hydroxyproline | 0.0018 | −1.49 | Down |

Table 37B indicates MS/MS analysis of a Day 1 serum sample for prediction of outcome GOSE at 12-month for 15 metabolites.

TABLE 37B
MS/MS Day 1, GOSE 12-month (15 metabolites)

| | | Fold | Poor/Good |
|---|---|---|---|
| Metabolites | p-value | Change | outcome |
| C3 | 0.085 | 1.27 | Up |
| Ornithine | 0.0581 | 1.36 | Up |
| C0 | 0.504 | 1.31 | Up |
| SM 16:1 OH | 0.462 | 1.05 | Up |
| LYSOC14:0 | 0.088 | 1.19 | Up |
| LYSOC20:3 | 0.234 | 1.13 | Up |
| Homocysteine | 0.344 | −1.09 | Up |
| Serotonin | 0.084 | −1.27 | Down |
| C9 | 0.133 | −1.21 | Down |
| PC ae 36:0 | 0.047 | −1.27 | Down |
| Methionine-sulfoxide | 0.0221 | −1.42 | Down |
| Serine | 0.0347 | −1.23 | Down |
| Adimethylarginine | 0.0318 | −1.35 | Down |
| Spermine | 0.0027 | −1.38 | Down |
| trans-Hydroxyproline | 0.0018 | −1.49 | Down |

Regarding Tables 37A and 37B, a Multivariate Data Analysis (OPLS-DA/PLS-DA) for Day 1 MS/MS analysis (Poor vs. Good outcome 12-months) GOSE 12-month is represented in Table 37C, showing (*) optimized prediction using 21 and 15 metabolites, versus 43, 34, 29, or 23 metabolites.

TABLE 37C
Multivariate Data Analysis (OPLS-DA/PLS-DA)
Day 1 MS/MS analysis
(Poor vs. Good outcome 12-months)

| Analytical | Sampling | | | | | | | | | |
|---|---|---|---|---|---|---|---|---|---|---|
| Platforms | Time | R2 | Q2 | p value | Sensitivity | Specificity | AUROC | VIP > | Metabolites | Patients |
| MS/MS | Day 1 | 0.832 | 0.558 | 0.0011 | 100 | 100 | 0.99 | 1 | 43 | Poor |
| | Day 1 | 0.81 | 0.5 | 0.002 | 100 | 100 | 1 | 1.1 | 34 | outcome, |
| | Day 1 | 0.824 | 0.548 | 0.0009 | 100 | 100 | 1 | 1.2 | 29 | n = 14 |
| | Day 1 | 0.799 | 0.556 | 0.0006 | 100 | 100 | 1 | 1.3 | 23 | Good |
| | Day 1* | 0.881 | 0.584 | 0.0002 | 100 | 100 | 0.99 | 1.4 | 21* | outcome |
| | Day 1* | 0.571 | 0.373 | 0.002 | 100 | 86 | 0.98 | 1.5 | 15* | n = 15 |

Table 38A indicates MS/MS metabolite analysis in a Day 4 serum sample, for prediction of GOSE 12-month for 18 metabolites.

TABLE 38A
MS/MS Day 4, GOSE 12-month (18 metabolites)

| | | Fold | Poor/Good |
|---|---|---|---|
| Metabolites | p-value | Change | outcome |
| C6 | 0.151 | 1.36 | Up |
| C3 OH | 0.198 | 1.16 | Up |
| C18:1 OH | 0.532 | 1.06 | Up |
| Tryptophan | 0.0678 | 1.32 | Up |
| C3:1 | 0.151 | 1.25 | Up |
| Tyrosine | 0.0362 | 1.18 | Up |
| Creatinine | 0.041 | 1.36 | Up |
| LysoPC 14:0 | 0.0911 | 1.16 | Up |
| Alanine | 0.176 | 1.17 | Up |
| C16 | 0.225 | −1.16 | Up |
| C2 | 0.186 | −1.61 | Down |
| C14 | 0.059 | −1.42 | Down |
| Beta-hydroxy butyric | 0.186 | −10.21 | Down |
| Spermine | 0.079 | −1.19 | Down |
| Betaine | 0.0238 | −1.65 | Down |
| C14:2 | 0.0066 | −1.34 | Down |
| Aspartic acid | 0.0457 | −1.79 | Down |
| C18 | 0.005 | −1.58 | Down |

Table 38B indicates MS/MS Day 4, GOSE 12-month for 13 metabolites.

TABLE 38B
MS/MS Day 4, GOSE 12-month (13 metabolites)

| | | Fold | Poor/Good |
|---|---|---|---|
| Metabolites | p-value | Change | outcome |
| C6 | 0.151 | 1.36 | Up |
| C3 OH | 0.198 | 1.16 | Up |
| Tryptophan | 0.0678 | 1.32 | Up |
| C3:1 | 0.151 | 1.25 | Up |
| Tyrosine | 0.0362 | 1.18 | Up |
| Creatinine | 0.041 | 1.36 | Up |
| LysoPC 14:0 | 0.0911 | 1.16 | Up |
| C14 | 0.059 | −1.42 | Down |
| Beta-hydroxy butyric | 0.186 | −10.21 | Down |
| Betaine | 0.0238 | −1.65 | Down |
| C14:2 | 0.0066 | −1.34 | Down |
| Aspartic acid | 0.0457 | −1.79 | Down |
| C18 | 0.005 | −1.58 | Down |

Regarding Tables 38A and 38B, a Multivariate Data Analysis (OPLS-DA/PLS-DA) for Day 4 MS/MS analysis (Poor vs. Good outcome 12-months) GOSE 12-month is represented in Table 38C, showing (*) optimized prediction using 18 and 13 metabolites, versus using 53, 39, 29, or 26 metabolites.

TABLE 38C
Multivariate Data Analysis (OPLS-DA/PLS-DA)
(Poor vs. Good outcome 12-months)

| Analytical | Sampling | | | | | | | | | |
|---|---|---|---|---|---|---|---|---|---|---|
| Platforms | Time | R2 | Q2 | p value | Sensitivity | Specificity | AUROC | VIP > | Metabolites | Patients |
| MS/MS | Day 4 | 0.75 | 0.488 | 0.00015 | 85 | 88 | 0.99 | 1 | 53 | Poor outcome, |
| | Day 4 | 0.793 | 0.593 | 0.0004 | 93 | 100 | 1 | 1.1 | 39 | n = 13 |
| | Day 4 | 0.792 | 0.624 | 0.0004 | 100 | 100 | 1 | 1.2 | 29 | Good outcome |
| | Day 4 | 0.779 | 0.583 | 0.0003 | 77 | 100 | 0.98 | 1.3 | 26 | n = 13 |
| | Day 4* | 0.707 | 0.497 | 0.0023 | 93 | 100 | 1 | 1.4 | 18* | |
| | Day 4* | 0.559 | 0.488 | 0.0004 | 86 | 100 | 0.97 | 1.5 | 13* | |

Table 39A indicates NMR Day 1, GOSE 3-month for 12 metabolites.

TABLE 39A
NMR Day 1, GOSE 3-month (12 metabolites)

| | | Fold | Poor/Good |
|---|---|---|---|
| Metabolites | p-value | Change | outcome |
| Ornithine | 0.218 | 1.21 | Up |
| Glucose | 0.0868 | 1.23 | Up |
| Acetone | 0.0501 | 2.37 | Up |
| Lactate | 0.0138 | 1.56 | Up |
| Glycerol | 0.0115 | 1.79 | Up |
| Betaine | 0.04 | 1.56 | Up |
| Choline | 0.0402 | 1.32 | Up |
| Serine | 0.0232 | 1.38 | Up |
| Glycine | 0.0345 | 1.43 | Up |
| Formate | 0.156 | 1.21 | Up |
| Isoleucine | 0.0538 | −1.6 | Down |
| Dimethylamine | 0.389 | −1 | Down |

Table 39B indicates NMR Day 1, GOSE 3-month for 6 metabolites.

TABLE 39B
NMR Day 1, GOSE 3-month (6 metabolites)

| | | Fold | Poor/Good |
|---|---|---|---|
| Metabolites | p-value | Change | outcome |
| Ornithine | 0.218 | 1.21 | Up |
| Acetone | 0.0501 | 2.37 | Up |
| Lactate | 0.0138 | 1.56 | Up |
| Glycerol | 0.0115 | 1.98 | Up |
| Betaine | 0.04 | 1.43 | Up |
| Choline | 0.0402 | 1.32 | Up |

Regarding Tables 39A and 39B, a Multivariate Data Analysis (OPLS-DA/PLS-DA) for Day 1 NMR analysis (Poor vs. Good outcome 3-months) GOSE 3-month is represented in Table 39C, showing (*) optimized prediction using 12 and 6 metabolites, versus using 22, 14, or 10 metabolites.

TABLE 39C
Multivariate Data Analysis (OPLS-DA/PLS-DA)
(Poor vs. Good outcome 3-months)

| Analytical | Sampling | | | p | | | | | | |
|---|---|---|---|---|---|---|---|---|---|---|
| Platforms | Time | R2 | Q2 | value | Sensitivity | Specificity | AUROC | VIP > | Metabolites | Patients |
| NMR | Day 1 | 0.49 | 0.21 | 0.026 | 100 | 0.87 | 0.99 | 1.0 | 22 | Poor |
| | Day 1 | 0.465 | 0.192 | 0.053 | 75 | 100 | 0.99 | 1.1 | 14 | outcome, |
| | Day 1* | 0.474 | 0.18 | 0.067 | 89 | 100 | 0.97 | 1.2 | 12 | n = 35 |
| | Day 1 | 0.47 | 0.25 | 0.017 | 72 | 100 | 0.92 | 1.4 | 10 | Good |
| | Day 1* | 0.414 | 0.151 | 0.194 | 83 | 89 | 0.96 | 1.5 | 6 | outcome |
| | | | | | | | | | | n = 9 |

Table 40A indicates NMR Day 4, GOSE 3-month for 9 metabolites.

TABLE 40A
NMR Day 4, GOSE 3-month (9 metabolites)

| | | Fold | Poor/Good |
|---|---|---|---|
| Metabolites | p-value | Change | outcome |
| Valine | 0.0018 | 1.39 | Up |
| N-Acetylaspartate | 0.0093 | 1.46 | Up |
| Tyrosine | 0.003 | 1.45 | Up |
| Lysine | 0.03 | 1.38 | Up |
| Histidine | 0.0134 | 1.2 | Up |
| Dimethyl Sulfone | 0.0596 | 5.88 | Up |
| Pyruvate | 0.732 | −1.05 | Down |
| Taurine | 0.715 | −1.06 | Down |
| Gluoconate | 0.009 | −2.25 | Down |

Table 40B indicates NMR Day 4, GOSE 3-month for 6 metabolites.

TABLE 40B
NMR Day 4, GOSE 3-month (6 metabolites)

| | | Fold | Poor/Good |
|---|---|---|---|
| Metabolites | p-value | Change | outcome |
| Valine | 0.0018 | 1.39 | Up |
| N-Acetylaspartate | 0.0093 | 1.46 | Up |
| Tyrosine | 0.003 | 1.45 | Up |
| Lysine | 0.03 | 1.38 | Up |
| Taurine | 0.715 | −1.06 | Down |
| Gluconate | 0.009 | −2.25 | Down |

Regarding Tables 40A and 40B, a Multivariate Data Analysis (OPLS-DA/PLS-DA) for Day 4 NMR analysis (Poor vs. Good outcome 3-months) GOSE 3-month is represented in Table 40C, showing (*) optimized prediction using 9 and 6 metabolites, versus using 26, 19, 16, and 10 metabolites.

TABLE 40C
Multivariate Data Analysis (OPLS-DA/PLS-DA)-NMR
(Poor vs. Good outcome 3-months)

| Analytical | Sampling | | | | | | | VIP | | |
|---|---|---|---|---|---|---|---|---|---|---|
| Platforms | Time | R2 | Q2 | p value | Sensitivity | Specificity | AUROC | > | Metabolites | Patients |
| NMR | Day 4 | 0.75 | 0.52 | 0.0016 | 99 | 90 | 0.99 | 1 | 26 | Poor |
| | Day 4 | 0.727 | 0.52 | 0.0012 | 100 | 100 | 1 | 1.1 | 19 | outcome, |
| | Day 4 | 0.743 | 0.541 | 0.0007 | 100 | 100 | 1 | 1.2 | 16 | n = 23 |
| | Day 4 | 0.75 | 0.576 | 0.0002 | 100 | 100 | 1 | 1.3 | 10 | Good |
| | Day 4* | 0.751 | 0.595 | 0.0001 | 100 | 96 | 1 | 1.4 | 9* | outcome |
| | Day 4* | 0.651 | 0.575 | 1.4 × 2-10⁻⁵ | 100 | 75 | 0.9 | 1.5 | 6* | n = 8 |

Table 41A indicates NMR Day 1, GOSE 12-month for 8 metabolites.

TABLE 41A
NMR Day 1, GOSE 12-month (8 metabolites)

| | | Fold | Poor/Good |
|---|---|---|---|
| Metabolites | p-value | Change | outcome |
| Ornithine | 0.028 | 1.65 | Up |
| Valine | 0.532 | 1.07 | Up |
| Succinate | 0.477 | 2.35 | Up |
| Leucine | 0.948 | 1.09 | Up |
| Gluconate | 0.273 | 4.04 | Up |
| Alanine | 0.048 | 1.35 | Up |
| Mannose | 0.298 | −1.15 | Down |
| 3-Hyroxyisovalerate | 0.029 | −1.68 | Down |

Table 41B indicates NMR Day 1, GOSE 12-month for 5 metabolites.

TABLE 41B
NMR Day 1, GOSE 12-month (5 metabolites)

| | | Fold | Poor/Good |
|---|---|---|---|
| Metabolites | p-value | Change | outcome |
| Ornithine | 0.028 | 1.65 | Up |
| Succinate | 0.532 | 2.35 | Up |
| Gluconate | 0.273 | 4.04 | Up |
| Alanine | 0.048 | −1.15 | Down |
| 3-Hyroxyisovalerate | 0.029 | −1.68 | Down |

Regarding Tables 41A and 41B, a Multivariate Data Analysis (OPLS-DA/PLS-DA) for Day 1 NMR analysis (Poor vs. Good outcome 12-months) GOSE 12-month is represented in Table 41C, showing (*) optimized prediction using 8 and 5 metabolites, versus using 26, 19, 16, and 12 metabolites.

TABLE 41C
Multivariate Data Analysis (OPLS-DA/PLS-DA)
(Poor vs. Good outcome 12-months)-Day 1-NMR

| Analytical | Sampling | | | p | | | | | | |
|---|---|---|---|---|---|---|---|---|---|---|
| Platforms | Time | R2 | Q2 | value | Sensitivity | Specificity | AUROC | VIP > | Metabolites | Patients |
| NMR | Day 1 | 0.731 | 0.463 | 0.003 | 73 | 93 | 0.92 | 1.0 | 26 | Poor outcome, |
| | Day 1 | 0.634 | 0.345 | 0.028 | 91 | 95 | 0.98 | 1.1 | 19 | n = 14 |
| | Day 1 | 0.636 | 0.386 | 0.015 | 83 | 91 | 0.94 | 1.2 | 16 | Good outcome |
| | Day 1 | 0.645 | 0.463 | 0.003 | 76 | 91 | 0.91 | 1.3 | 12 | n = 15 |
| | Day 1* | 0.543 | 0.348 | 0.03 | 93 | 86 | 0.96 | 1.4 | 8 | |
| | Day 1* | 0.519 | 0.361 | 0.02 | 100 | 100 | 1 | 1.5 | 5 | |

Table 42A indicates NMR Day 4, GOSE 12-month for 9 metabolites.

TABLE 42A
NMR Day 4, GOSE 12-month (9 metabolites)

| | | Fold | Poor/Good |
|---|---|---|---|
| Metabolites | p-value | Change | outcome |
| Dimethyl sulfone | 0.0178 | 9.7 | Up |
| Tyrosine | 0.0156 | 1.36 | Up |
| Hisitidine | 0.106 | 1.2 | Up |
| Valine | 0.0193 | 1.31 | Up |
| Leucine | 0.088 | 1.2 | Up |
| Taurine | 0.387 | 1.17 | Up |
| Hypoxanthine | 0.891 | −1.02 | Down |
| Isopropanol | 0.41 | −1.6 | Down |
| Beta-alanine | 0.166 | −1.23 | Down |

Table 42B indicates NMR Day 4, GOSE 12-month for 5 metabolites.

TABLE 42B
NMR Day 4, GOSE 12-month (5 metabolites)

| | | Fold | Poor/Good |
|---|---|---|---|
| Metabolites | p-value | Change | outcome |
| Dimethyl sulfone | 0.0178 | 9.7 | Up |
| Tyrosine | 0.0156 | 1.36 | Up |
| Valine | 0.0193 | 1.31 | Up |
| Hypoxanthine | 0.891 | −1.02 | Down |
| Beta-alanine | 0.166 | −1.23 | Down |

Regarding Tables 42A and 42B, a Multivariate Data Analysis (OPLS-DA/PLS-DA) for Day 4 NMR analysis (Poor vs. Good outcome 12-months) GOSE 12-month is represented in Table 42C, showing (*) optimized prediction using 9 and 5 metabolites, versus using 24, 19, and 12 metabolites.

TABLE 42C
Multivariate Data Analysis (OPLS-DA/PLS-DA)
(Poor vs. Good outcome 12-months)-NMR-Day 4

| Analytical | Sampling | | | p | | | | VIP | | |
|---|---|---|---|---|---|---|---|---|---|---|
| Platforms | Time | R2 | Q2 | value | Sensitivity | Specificity | AUROC | > | Metabolites | Patients |
| NMR | Day 4 | 0.691 | 0.396 | 0.029 | 94 | 92 | 0.95 | 1 | 24 | Poor outcome, |
| | Day 4 | 0.7 | 0.423 | 0.018 | 93 | 94 | 0.96 | 1.1 | 19 | n = 13 |
| | Day 4 | 0.708 | 0.339 | 0.056 | 100 | 100 | 1 | 1.2 | 12 | Good outcome |
| | Day 4* | 0.701 | 0.408 | 0.044 | 100 | 100 | 1 | 1.3 | 9 | n = 13 |
| | Day 4 | 0.651 | 0.364 | 0.16 | 71 | 100 | 1 | 1.4 | 6 | |
| | Day 4* | 0.631 | 0.394 | 0.31 | 100 | 100 | 1 | 1.5 | 5 | |

Table 43A indicates MS/MS Day 1, Mortality for 26 metabolites.

TABLE 43A
MS/MS Day 1, Mortality (26 metabolites)

| | | Fold | Poor/Good |
|---|---|---|---|
| Metabolites | p-value | Change | outcome |
| C3:1 | 0.0532 | 1.17 | Up |
| PC aa 38:0 | 0.0364 | 1.11 | Up |
| Glucose | 0.153 | 1.12 | Up |
| PC ae 40:6 | 0.455 | 1.06 | Up |
| C10:1 | 0.215 | 1.1 | Up |
| C14:1 | 0.272 | 1.54 | Up |
| C14 | 0.455 | 1.24 | Up |
| C10 | 0.262 | 1.41 | Up |
| C16:2 | 0.529 | 1.39 | Up |
| C8 | 0.852 | 1.27 | Up |
| C12 | 0.962 | 1.15 | Up |
| Citrulline | 0.027 | −1.31 | Down |
| C10:2 | 0.352 | −1.08 | Down |
| Leucine | 0.0261 | −1.42 | Down |
| Valine | 0.352 | −1.28 | Down |
| Isoleucine | 0.352 | −1.43 | Down |
| Histidine | 0.352 | −1.17 | Down |
| C16 OH | 0.161 | −1.07 | Down |
| Glutamine | 0.0068 | −1.25 | Down |

Table 43B indicates MS/MS Day 1, Mortality for 19 metabolites.

TABLE 43B
MS/MS Day 1, Mortality (11 metabolites)

| | | Fold | Poor/Good |
|---|---|---|---|
| Metabolites | p-value | Change | outcome |
| C3:1 | 0.0532 | 1.17 | Up |
| PC aa 38:0 | 0.0364 | 1.11 | Up |
| Glucose | 0.153 | 1.12 | Up |
| C16:2 | 0.529 | 1.39 | Up |
| Leucine | 0.0261 | −1.42 | Down |
| C10:2 | 0.352 | −1.08 | Down |
| Valine | 0.352 | −1.28 | Down |
| Isoleucine | 0.352 | −1.43 | Down |
| Histidine | 0.352 | −1.17 | Down |
| C16 OH | 0.161 | −1.07 | Down |
| Glutamine | 0.0068 | −1.25 | Down |

Regarding Tables 43A and 43B, a Multivariate Data Analysis (OPLS-DA/PLS-DA) for Day 1 MS/MS mortality analysis (Died vs. Survived) is represented in Table 43C, showing (*) optimized prediction using 26, 19, and 11 metabolites, versus using 48, 39, and 32 metabolites.

TABLE 43C
Multivariate Data Analysis (OPLS-DA/PLS-DA)
(Mortality)-Day 1-MS/MS

| Analytical | Sampling | | | | | | | | | |
|---|---|---|---|---|---|---|---|---|---|---|
| Platforms | Time | R2 | Q2 | p value | Sensitivity | Specificity | AUROC | VIP > | Metabolites | Patients |
| MS/MS | Day 1 | 0.684 | 0.429 | 0.00017 | 95 | 100 | 0.96 | 1 | 48 | Died |
| | Day 1 | 0.679 | 0.427 | 0.00023 | 88 | 94 | 0.94 | 1.1 | 39 | N = 21 |
| | Day 1 | 0.626 | 0.377 | 0.0012 | 94 | 94 | 0.95 | 1.2 | 32 | Survived |
| | Day 1* | 0.628 | 0.424 | 0.00027 | 88 | 88 | 0.98 | 1.3 | 26* | N = 23 |
| | Day 1* | 0.536 | 0.347 | 0.0022 | 79 | 100 | 0.98 | 1.4 | 19* | |
| | Day 1* | 0.48 | 0.257 | 0.017 | 82 | 75 | 0.91 | 1.5 | 11* | |

Table 44A indicates MS/MS Day 4. Mortality for 22 metabolites

TABLE 44A
MS/MS Day 4, Mortality (22 metabolites)

| | | Fold | Poor/Good |
|---|---|---|---|
| Metabolites | p-value | Change | outcome |
| Indole acetic acid | 0.0141 | 2.12 | Up |
| Alpha-Ketglutaric acid | 0.064 | 1.43 | Up |
| Hippric acid | 0.0927 | 2.28 | Up |
| C16:2 OH | 0.0697 | 1.16 | Up |
| Ornithine | 0.0541 | 1.23 | Up |
| PC aa 36:0 | 0.0284 | 1.3 | Up |
| C3 | 0.11 | 1.28 | Up |
| Threonine | 0.06 | 1.3 | Up |
| Alpha-Aminoadipic acid | 0.027 | 1.29 | Up |
| PC aa 38:0 | 0.0629 | 1.23 | Up |
| Tyrosine | 0.0887 | 1.19 | Up |
| Valine | 0.0484 | 1.53 | Up |
| Tryptophan | 0.055 | 1.21 | Up |
| C2 | 0.235 | −1.35 | Down |
| C8 | 0.261 | −1.46 | Down |
| C12:1 | 0.119 | −1.16 | Down |
| Betaine | 0.0589 | −1.34 | Down |
| C6 | 0.0027 | −1.48 | Down |
| Glutamine | 0.161 | −1.12 | Down |
| Taurine | 0.029 | −1.41 | Down |
| LysoPC 26:0 | 0.0486 | −1.37 | Down |

Table 44B indicates MS/MS Day mortality for 16 metabolites.

TABLE 44B
MS/MS Day 4, Mortality (16 metabolites)

| | | Fold | Poor/Good |
|---|---|---|---|
| Metabolites | p-value | Change | outcome |
| Alpha-Ketoglutaric acid | 0.064 | 1.43 | Up |
| C16:2 OH | 0.0697 | 1.16 | Up |
| Hippuric acid | 0.0927 | 2.28 | Up |
| Indole acetic acid | 0.0141 | 2.12 | Up |
| PC aa 36:0 | 0.0284 | 1.3 | Up |
| Ornithine | 0.0541 | 1.23 | Up |
| PC aa 38:0 | 0.0629 | 1.23 | Up |
| Alpha-Aminoadipic acid | 0.027 | 1.29 | Up |
| Tryptophan | 0.055 | 1.21 | Up |
| Valine | 0.0484 | 1.19 | Down |
| Leucine | 0.131 | 1.28 | Down |
| C12:1 | 0.119 | −1.16 | Down |
| C6 | 0.0027 | −1.48 | Down |
| Glutamine | 0.161 | −1.12 | Down |
| LysoPC 26:0 | 0.0486 | −1.37 | Down |
| Taurine | 0.029 | −1.41 | Down |

Regarding Tables 44A and 44B, a Multivariate Data Analysis (OPLS-DA/PLS-DA) for Day 4 MS/MS mortality analysis (Died vs. Survived) is represented in Table 44C, showing (*) optimized prediction using 22 and 16 metabolites, versus using 45, 35, 29, and 26 metabolites.

TABLE 44C
Multivariate Data Analysis (OPLS-DA/PLS-DA)
(Poor vs. Good outcome 3-month) MS/MS-Day 4

| Analytical | Sampling | | | | | | | | | |
|---|---|---|---|---|---|---|---|---|---|---|
| Platforms | Time | R2 | Q2 | p value | Sensitivity | Specificity | AUROC | VIP > | Metabolites | Patients |
| MS/MS | Day 4 | 0.761 | 0.476 | 0.001 | 90 | 87 | 0.95 | 1 | 45 | Died |
| | Day 4 | 0.779 | 0.491 | 0.001 | 90 | 83 | 1 | 1.1 | 35 | N = 12 |
| | Day 4 | 0.777 | 0.491 | 0.0006 | 100 | 100 | 1 | 1.2 | 29 | Survived |
| | Day 4 | 0.792 | 0.542 | 0.0003 | 100 | 100 | 1 | 1.3 | 26 | N = 19 |
| | Day 4* | 0.775 | 0.499 | 0.0009 | 100 | 100 | 1 | 1.4 | 22* | |
| | Day 4* | 0.757 | 0.505 | 0.0006 | 100 | 100 | 1 | 1.5 | 16* | |

Table 45A indicates NMR Day 1 mortality for 17 metabolites.

TABLE 45A
NMR Day 1, Mortality (17 metabolites)

| | | Fold | Poor/Good |
|---|---|---|---|
| Metabolites | p-value | Change | outcome |
| Glucose | 0.018 | 1.23 | Up |
| Betaine | 0.1 | 1.47 | Up |
| 3-Hydroxyisovalerate | 0.157 | 1.54 | Up |
| Citrate | 0.139 | 1.32 | Up |
| O-Phosphocholine | 0.0414 | 1.51 | Up |
| Dimethyl Sulfone | 0.673 | 6.64 | Up |
| Formate | 0.0201 | 1.12 | Up |
| Fumarte | 0.0124 | 1.35 | Up |
| 2-Oxglutarate | 0.189 | 1.14 | Up |
| Pyruvate | 0.089 | 1.26 | Up |
| Lactate | 0.36 | −1.17 | Down |
| Valine | 0.112 | −1.13 | Down |
| Isoleucine | 0.0275 | −1.32 | Down |
| Leucine | 0.0414 | −1.28 | Down |
| Diemthylamine | 0.487 | −1.14 | Down |
| Glutamine | 0.0189 | −1.17 | Down |
| Histidine | 0.284 | −1.13 | Down |

Table 45B indicates NMR Day 1 Mortality for 5 metabolites.

TABLE 45B
NMR Day 1, Mortality (5 metabolites)

| | p- | Fold | Poor/Good |
|---|---|---|---|
| Metabolites | value | Change | outcome |
| Glucose | 0.018 | 1.23 | Up |
| Betaine | | 1.47 | Up |
| 3-Hydroxyisovalerate | 0.157 | 1.54 | Up |
| Citrate | 0.139 | 1.32 | Up |
| Lactate | 0.36 | −1.17 | Down |

Regarding Tables 45A and 45B, a Multivariate Data Analysis (OPLS-DA/PLS-DA) for Day 1 NMR mortality analysis (Died vs. Survived) is represented in Table 45C, showing (*) optimized prediction using 17 and 6 metabolites, versus using 21, 14, 11, and 10 metabolites.

TABLE 45C
Multivariate Data Analysis (OPLS-DA/PLS-DA)
(Mortality) NMR-Day 1

| Analytical | Sampling | | | | | | | | | |
|---|---|---|---|---|---|---|---|---|---|---|
| Platforms | Time | R2 | Q2 | p value | Sensitivity | Specificity | AUROC | VIP > | Metabolites | Patients |
| NMR | Day 1 | 0.607 | 0.281 | 0.007 | 85 | 75 | 0.95 | 1.0 | 21 | Died |
| | Day 1* | 0.498 | 0.243 | 0.01 | 84 | 87 | 0.88 | 1.1 | 17* | N = 21 |
| | Day 1 | 0.496 | 0.265 | 0.01 | 95 | 63 | 0.91 | 1.2 | 14 | Survived |
| | Day 1 | 0.514 | 0.26 | 0.014 | 58 | 94 | 0.79 | 1.3 | 11 | N = 23 |
| | Day 1 | 0.46 | 0.228 | 0.033 | 77 | 80 | 0.85 | 1.4 | 10 | |
| | Day 1* | 0.417 | 0.239 | 0.025 | 86 | 81 | 89 | 1.5 | 6* | |

Table 46A indicates NMR Day 4 mortality for 16 metabolites

TABLE 46A
NMR Day 4, Mortality (16 metabolites)

| | p- | Fold | Poor/Good |
|---|---|---|---|
| Metabolites | value | Change | outcome |
| Isobutyrate | 0.685 | 1.3 | Up |
| Creatine | 0.163 | 1.39 | Up |
| Creatinine | 0.564 | 1.17 | Up |
| Valine | 0.49 | 1.24 | Up |
| Lysine | 0.0507 | 1.35 | Up |
| Asparagine | 0.424 | 1.19 | Up |
| Leucine | 0.754 | 1.27 | Up |
| Tyrosine | 0.837 | 1.17 | Up |
| 2-Aminobutyrate | 0.0961 | 1.3 | Up |
| 4-Hydroxybutyrate | 0.723 | −2 | Down |
| Methionine | 0.452 | −1.08 | Down |
| Urea | 0.721 | −1.87 | Down |
| Hypoxanthine | 0.013 | −1.26 | Down |
| Taurine | 0.07 | −1.29 | Down |
| Gluconate | 0.0077 | −2.19 | Down |
| Betaine | 0.0261 | −1.35 | Down |

Table 46B indicates NMR Day 4 Mortality for 8 metabolites.

TABLE 46B
NMR Day 4, Mortality (8 metabolites)

| | p- | Fold | Poor/Good |
|---|---|---|---|
| Metabolites | value | Change | outcome |
| Isobutyrate | 0.685 | 1.3 | Up |
| Valine | 0.49 | 1.24 | Up |
| Lysine | 0.0507 | 1.35 | Up |
| 2-Aminobutyrate | 0.0961 | 1.3 | Up |
| Hypoxanthine | 0.013 | −1.26 | Down |
| Taurine | 0.07 | −1.29 | Down |
| Gluconate | 0.0077 | −2.19 | Down |
| Betaine | 0.0261 | −1.35 | Down |

Table 46C indicates NMR analysis of Day 4 blood sample as a prediction of Mortality based on 5 metabolites.

TABLE 46C
NMR Day 4, Mortality (5 metabolites)

| Metabolites | p-value | Fold Change | Poor/Good outcome |
|---|---|---|---|
| Valine | 0.49 | 1.24 | Up |
| Lysine | 0.0507 | 1.35 | Up |
| Taurine | 0.07 | −1.29 | Down |
| Gluconate | 0.0077 | −2.19 | Down |
| Betaine | 0.0261 | −1.35 | Down |

Regarding Tables 46A, 46B, and 46C, a Multivariate Data Analysis (OPLS-DA/PLS-DA) for Day 4 NMR mortality analysis (Died vs. Survived) is represented in Table 46D, showing (*) optimized prediction using 16, 8, and 5 metabolites, versus using 23, 19, and 7 metabolites.

TABLE 46D
Multivariate Data Analysis (OPLS-DA/PLS-DA)
(Mortality)-NMR-Day 4

| Analytical | Sampling | | | | | | | | | |
|---|---|---|---|---|---|---|---|---|---|---|
| Platforms | Time | R2 | Q2 | p value | Sensitivity | Specificity | AUROC | VIP > | Metabolites | Patients |
| NMR | Day 4 | 0.662 | 0.464 | 0.0027 | 94 | 91 | 1 | 1 | 23 | Died |
| | Day 4 | 0.649 | 0.468 | 0.0025 | 100 | 71 | 1 | 1.1 | 19 | N = 12 |
| | Day 4* | 0.608 | 0.393 | 0.011 | 91 | 90 | 0.96 | 1.2 | 16* | Survived |
| | Day 4* | 0.546 | 0.387 | 0.022 | 84 | 93 | 1 | 1.3 | 8* | N = 19 |
| | Day 4 | 0.546 | 0.375 | 0.015 | 86 | 76 | 0.95 | 1.4 | 7 | |
| | Day 4* | 0.538 | 0.42 | 0.0.38 | 75 | 93 | 0.89 | 1.5 | 5* | |

The metabolite optimization modeling represented from Table 35A to Table 46D indicates that predictions based on a Day 4 or Day 1 serum samples can effectively determine outcomes with reliable sensitivity and specificity. An optimized multivariate analysis can be tailored to the analytical platform of either MS/MS or NMR, using as few metabolite variables as possible, based on metabolites capable of being measured on the selected analytical platform. Outcomes regarding the GOSE parameter (at 3-months or 12-months) or mortality are valuable to know when a patient presents with a severe traumatic brain injury.

### REFERENCES

1. Roozenbeek B, Maas A I, Menon D K. Changing patterns in the epidemiology of traumatic brain injury. Nature reviews. Neurology. 2013; 9 (4): 231-236.
2. Dewan M C, Rattani A, Gupta S, et al. Estimating the global incidence of traumatic brain injury. J Neurosurg. 2018:1-18.
3. Multi-Society Task Force on PVS. Medical aspects of the persistent vegetative state (2). N Engl J Med. 1994; 330 (22): 1572-1579.
4. Multi-Society Task Force on PVS. Medical aspects of the persistent vegetative state (1). N Engl J Med. 1994; 330 (21): 1499-1508.

5. Lannoo E, Van Rietvelde F, Colardyn F, et al. Early predictors of mortality and morbidity after severe closed head injury. J Neurotrauma. 2000; 17 (5): 403-414.
6. Levati A, Farina M L, Vecchi G, Rossanda M, Marrubini M B. Prognosis of severe head injuries. J Neurosurg. 1982; 57 (6): 779-783.
7. Zygun D A, Laupland K B, Hader W J, et al. Severe traumatic brain injury in a large Canadian health region. Can J Neurol Sci. 2005; 32 (1): 87-92.
8. Greenwald B D, Ambrose A F, Armstrong G P. Mild brain injury. Rehabil Res Pract. 2012; 2012:469475.
9. Rees P M. Contemporary issues in mild traumatic brain injury. Arch Phys Med Rehabil. 2003; 84 (12): 1885-1894.
10. Jacobs B, Beems T, van der Vliet™, et al. Outcome prediction in moderate and severe traumatic brain injury: a focus on computed tomography variables. Neurocrit Care. 2013; 19 (1): 79-89.
11. Banoei M M, Casault C, Metwaly S M, Winston B W. Metabolomics and Biomarker Discovery in Traumatic Brain Injury. J Neurotrauma. 2018; 35 (16): 1831-1848.
12. Wilson J T, Pettigrew L E, Teasdale G M. Structured interviews for the Glasgow Outcome Scale and the extended Glasgow Outcome Scale: guidelines for their use. J Neurotrauma. 1998; 15 (8): 573-585.
13. Maas A I, Murray G, Henney H, 3rd, et al. Efficacy and safety of dexanabinol in severe traumatic brain injury: results of a phase III randomised, placebo-controlled, clinical trial. Lancet Neurol. 2006; 5 (1): 38-45.
14. Alali A S, Vavrek D, Barber J, Dikmen S, Nathens A B, Temkin N R. Comparative study of outcome measures and analysis methods for traumatic brain injury trials. J Neurotrauma. 2015; 32 (8): 581-589.
15. Mendez K M, Reinke S N, Broadhurst D I. A comparative evaluation of the generalised predictive ability of eight machine learning algorithms across ten clinical metabolomics data sets for binary classification. Metabolomics: Official journal of the Metabolomic Society. 2019; 15 (12): 150.
16. Perel P, Arango M, Clayton T, et al. Predicting outcome after traumatic brain injury: practical prognostic models based on large cohort of international patients. Bmj. 2008; 336 (7641): 425-429.
17. Hukkelhoven C, Steyerberg E, Rampen A, et al. Patient age and outcome following severe traumatic brain injury: an analysis of 5600 patients. Journal of neurosurgery. 2003; 99:666-673.
18. Tokutomi T, Miyagi T, Ogawa T, et al. Age-associated increases in poor outcomes after traumatic brain injury: a report from the Japan Neurotrauma Data Bank. J Neurotrauma. 2008; 25 (12): 1407-1414.
19. Lenell S, Nyholm L, Lewen A, Enblad P. Clinical outcome and prognostic factors in elderly traumatic brain injury patients receiving neurointensive care. Acta Neurochir (Wien). 2019; 161 (6): 1243-1254.
20. Mata-Mbemba D, Mugikura S, Nakagawa A, et al. Early CT findings to predict early death in patients with traumatic brain injury: Marshall and Rotterdam CT scoring systems compared in the major academic tertiary care hospital in northeastern Japan. Acad Radiol. 2014; 21 (5): 605-611.
21. Zhu G W, Wang F, Liu W G. Classification and prediction of outcome in traumatic brain injury based on computed tomographic imaging. The Journal of international medical research. 2009; 37 (4): 983-995.
22. Mahadewa T G B, Golden N, Saputra A, Ryalino C. Modified Revised Trauma-Marshall score as a proposed tool in predicting the outcome of moderate and severe traumatic brain injury. Open Access Emerg Med. 2018; 10:135-139.
23. Roozenbeek B, Lingsma H F, Lecky F E, et al. Prediction of outcome after moderate and severe traumatic brain injury: external validation of the International Mission on Prognosis and Analysis of Clinical Trials (IMPACT) and Corticoid Randomisation After Significant Head injury (CRASH) prognostic models. Crit Care Med. 2012; 40 (5): 1609-1617.
24. Hukkelhoven C, Steyerberg E, Habbema J, et al. Predicting Outcome after Traumatic Brain Injury: Development and Validation of a Prognostic Score Based on Admission Characteristics. Journal of neurotrauma. 2005; 22:1025-1039.
25. Roozenbeek B, Chiu Y-L, Lingsma H F, et al. Predicting 14-day mortality after severe traumatic brain injury: application of the IMPACT models in the brain trauma foundation TBI-Trac® New York State database. Journal of neurotrauma. 2012; 29 (7): 1306-1312.
26. Low D, Kuralmani V, Ng S K, Lee K K, Ng I, Ang B T. Prediction of outcome utilizing both physiological and biochemical parameters in severe head injury. J Neurotrauma. 2009; 26 (8): 1177-1182.
27. Zador Z, Sperrin M, King A T. Predictors of Outcome in Traumatic Brain Injury: New Insight Using Receiver Operating Curve Indices and Bayesian Network Analysis. PLOS One. 2016; 11 (7): e0158762.
28. Eriksson L, Antti H, Gottfries J, et al. Using chemometrics for navigating in the large data sets of genomics, proteomics, and metabonomics (gpm). Analytical and bioanalytical chemistry. 2004; 380 (3): 419-429.
29. Law S-H, Chan M-L, Marathe G K, Parveen F, Chen C-H, Ke L-Y. An Updated Review of Lysophosphatidylcholine Metabolism in Human Diseases. Int J Mol Sci. 2019; 20 (5): 1149.
30. Sugasini D, Thomas R, Yalagala P C R, Tai L M, Subbaiah P V. Dietary docosahexaenoic acid (DHA) as lysophosphatidylcholine, but not as free acid, enriches brain DHA and improves memory in adult mice. Scientific Reports. 2017; 7 (1): 11263.
31. Pasvogel A E, Miketova P, Moore I M. Differences in CSF phospholipid concentration by traumatic brain injury outcome. Biological research for nursing. 2010; 11 (4): 325-331.
32. Fiandaca M S, Mapstone M, Mahmoodi A, et al. Plasma metabolomic biomarkers accurately classify acute mild traumatic brain injury from controls. PLOS One. 2018; 13 (4): e0195318.
33. Kerr M E, Ilyas Kamboh M, Yookyung K, et al. Relationship between apoE4 allele and excitatory amino acid levels after traumatic brain injury. Crit Care Med. 2003; 31 (9): 2371-2379.
34. Wagner A K, Fabio A, Puccio A M, et al. Gender associations with cerebrospinal fluid glutamate and lactate/pyruvate levels after severe traumatic brain injury. Crit Care Med. 2005; 33 (2): 407-413.
35. Amorini A M, Lazzarino G, Di Pietro V, et al. Severity of experimental traumatic brain injury modulates changes in concentrations of cerebral free amino acids. Journal of cellular and molecular medicine. 2017; 21 (3): 530-542.
36. Aquilani R, Iadarola P, Boschi F, Pistarini C, Arcidiaco P, Contardi A. Reduced plasma levels of tyrosine, precursor of brain catecholamines, and of essential amino acids in patients with severe traumatic brain injury after rehabilitation. Archives of physical medicine and rehabilitation. 2003; 84 (9): 1258-1265.
37. Yan E B, Frugier T, Lim C K, et al. Activation of the kynurenine pathway and increased production of the excitotoxin quinolinic acid following traumatic brain injury in humans. Journal of neuroinflammation. 2015; 12:110-110.
38. Sinz E H, Kochanek P M, Heyes M P, et al. Quinolinic Acid is Increased in CSF and Associated with Mortality after Traumatic Brain Injury in Humans. Journal of Cerebral Blood Flow & Metabolism. 1998; 18 (6): 610-615.
39. Dumas M E, Davidovic L. Metabolic Profiling and Phenotyping of Central Nervous System Diseases: Metabolites Bring Insights into Brain Dysfunctions. J Neuroimmune Pharmacol. 2015; 10 (3): 402-424.
40. Shi J, Dong B, Mao Y, et al. Review: Traumatic brain injury and hyperglycemia, a potentially modifiable risk factor. Oncotarget. 2016; 7 (43): 71052-71061.
41. Carpenter K L, Jalloh I, Hutchinson P J. Glycolysis and the significance of lactate in traumatic brain injury. Front Neurosci. 2015; 9:112.
42. Brooks G A, Martin N A. Cerebral metabolism following traumatic brain injury: new discoveries with implications for treatment. Front Neurosci. 2014; 8:408.
43. Wishart D S, Knox C, Guo A C, et al. HMDB: a knowledgebase for the human metabolome. Nucleic Acids Research 2009; 37 (Database): D603-D10.
44. Weljie A M, Newton J, Mercier P, Carlson E, Slupsky C M. Targeted profiling: quantitative analysis of 1H NMR metabolomics data. Anal Chem 2006; 78 (13): 4430-42.
45. Nicholson J K, Foxall P J, Spraul M, Farrant R D, Lindon J C. 750 MHz 1H and 1H-13C NMR spectroscopy of human blood plasma. Anal Chem 1995; 67 (5): 793-811.
46. Weljie A M, Dowlatabadi R, Miller B J, Vogel H J, Jirik F R. An Inflammatory Arthritis-Associated Metabolite Biomarker Pattern Revealed by ¹H NMR Spectroscopy. Journal of Proteome Research 2007; 6 (9): 3456-64.
47. Banoei M M, Vogel H J, Weljie A M, Yende S, Angus D C, Winston B W. Plasma lipid profiling for the prognosis of 90-day mortality, in-hospital mortality, ICU admission, and severity in bacterial community-acquired pneumonia (CAP). Crit Care 2020; 24 (1): 461.
48. Eriksson L, Trygg J, Wold S. CV-ANOVA for significance testing of PLS and OPLS® models. Journal of Chemometrics 2008; 22 (11-12): 594-600.
49. de Jong S. SIMPLS: An alternative approach to partial least squares regression. Chemometrics and Intelligent Laboratory Systems 1993; 18 (3): 251-63.
50. Goodacre R, Vaidyanathan S, Dunn W B, Harrigan G G, Kell D B. Metabolomics by numbers: acquiring and understanding global metabolite data. Trends Biotechnol 2004; 22 (5): 245-52.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A method of determining a likelihood of an unfavourable outcome in a subject having a severe traumatic brain injury (sTBI), comprising:
obtaining a sample from a subject at day 1 and/or day 4 post sTBI,
measuring an amount of a plurality of metabolites in the sample, and
comparing levels of the plurality of metabolites in the sample with a control;
wherein an outcome of the comparing step is an increase or decrease in quantity of said plurality of metabolites in the sample relative to the control, and
wherein unfavourable outcome is determined as likely when:
in the day 1 sample, at least two, or at least 5, of the following metabolites of the plurality of metabolites are changed relative to the control: C3:1, LYSOC17:0, LYSOC18:0, LYSOC16:0, C18:2, LYSOC18:1, C14, C18:1, C18, C16, tyrosine, homocysteine, C3, CO, C4, ornithine, LYSOC14:0, SM 16:1 OH, LYSOC20:3, LYSOC28:1, phenylalanine, glutamine, PC ae 36:0, histidine, SM 20:2, isoleucine, citrulline, methionine-sulfoxide, asymmetric dimethylarginine, C5OH, C10:2, acetyl-ornithine, C9, methionine-sulfoxide, spermine, serotonin, serine, trans-hydroxyproline, succinate, gluconate, acetone, lactate, glycerol, betaine, choline, alanine, and 3-hyroxyisovalerate;
and/or
in the day 4 sample, at least two, or at least 5, of the following metabolites of the plurality of metabolites are changed relative to the control: valine, N-acetylaspartate, tyrosine, lysine, dimethylsulfone, taurine, gluconate, hypoxanthine, beta-alanine, C3OH, glutamic acid, LYSOC18:0, PC36:0AA, C18:2, C3:1, C3, ornithine, CO, SM 16:1 OH, LYSOC14:0, LYSOC20:3, homocysteine, C16:1, glutamine, beta-hydroxybutyric acid, uric acid, serotonin, C9, PC ae 36:0, methionine-sulfoxide, serine, a-dimethylarginine, spermine, and trans-hydroxyproline;
wherein the unfavourable outcome comprises a Glasgow Outcome Scale Extended (GOSE) score≤4 from 3 months to 12 months post sTBI.

2. The method of claim 1, wherein said likelihood of said unfavorable outcome is determined for three months following the sTBI.

3. The method of claim 1, wherein said likelihood of said unfavorable outcome is determined for twelve months following the sTBI.

4. The method of claim 1, wherein said likelihood of said unfavorable outcome is determined from a Day 1 sample, with an increase or decrease in the at least two, or the at least 5, metabolites as per Tables 35A, 35B, 37A, or 37B:

| | |
|---|---|
| Lyso PC 17:0 | Up |
| Lyso PC 18:0 | Up |
| C3:1 | Up |
| Lyso PC 16:0 | Up |
| Lyso PC 18:1 | Up |
| C18:2 | Up |
| C14 | Up |
| C18 | Up |
| C18:1 | Up |
| C16 | Up |
| C14:2 | Up |
| Tyrosine | Down |
| Asparagine | Down |
| PC ae 36:0 | Down |
| C16:2 | Down |
| Phenylalanine | Down |
| C16:1 | Down |
| Glutamine | Down |
| SM 20:2 | Down |
| PC aa 32:2 | Down |
| Isoleucine | Down |
| Citrulline | Down |
| Histidine | Down |
| Glutamate | Down |
| Methionine-Sulfoxide | Down |
| Asymmetric | Down |
| dimethylargine | |
| | |
|---|---|
| LYSOC17:0 | Up |
| LYSOC18:0 | Up |
| LYSOC16:0 | Up |
| C18:2 | Up |
| C14 | Up |
| C18:1 | Up |
| C18 | Up |
| C16 | Up |
| Glutamine | Down |
| Histidine | Down |
| SM 20:2 | Down |
| Methionine-sulfoxide | Down |
| Asymmetric | Down |
| dimethylarginine | |
| | |
|---|---|
| C5OH | Down |
| Homocysteine | Up |
| C3 | Up |
| C0 | Up |
| C4 | Up |
| Ornithine | Up |
| LYSOC14:0 | Up |
| SM 16:1 OH | Up |
| LYSOC20:3 | Up |
| LYSOC28:1 | Up |
| C10:2 | Down |
| Acetyl-ornithine | Down |
| C9 | Down |
| Adimethylarginine | Down |
| Methionine-sulfoxide | Down |
| Spermine | Down |
| PC ae 36:0 | Down |
| Citrulline | Down |
| Serotonin | Down |
| Serine | Down |
| trans-Hydroxyproline | Down |
| | |
|---|---|
| C3 | Up |
| Ornithine | Up |
| C0 | Up |
| SM 16:1 OH | Up |
| LYSOC14:0 | Up |
| LYSOC20:3 | Up |
| Homocysteine | Up |
| Serotonin | Down |
| C9 | Down |
| PC ae 36:0 | Down |
| Methionine-sulfoxide | Down |
| Serine | Down |
| Adimethylarginine | Down |
| Spermine | Down |
| trans-Hydroxyproline | Down |

5. The method of claim 1, wherein said likelihood of said unfavorable outcome is determined from a Day 4 sample, with an increase or decrease in the at least two, or the at least 5, metabolites as per Tables 36A, 36B, 38A, or 38B:

| | |
|---|---|
| C3OH | Up |
| Glutamic acid | Up |
| LYSOC18:0 | Up |
| Ornithine | Up |
| PC aa3 6:0 | Up |
| C18:2 | Up |
| alpha-Aminoadipic acid | Up |
| Indole acetic acid | Up |
| C3:1 | Up |
| PC aa 40:2 | Down |
| C16:1 | Down |
| Serine | Down |
| Glutamine | Down |
| beta-Hydroxybutyric acid | Down |
| Uric acid | Down |
| | |
|---|---|
| C3OH | Up |
| Glutamic acid | Up |
| LYSOC18:0 | Up |
| Ornithine | Up |
| PC36:0AA | Up |
| C18:2 | Up |
| C3:1 | Up |
| C16:1 | Down |
| Glutamine | Down |
| beta-Hydroxybutyric acid | Down |
| Uric acid | Down |
| C6 | Up |
| C3 OH | Up |
| C18:1 OH | Up |
| Tryptophan | Up |
| C3:1 | Up |
| Tyrosine | Up |
| Creatinine | Up |
| LysoPC 14:0 | Up |
| Alanine | Up |
| C16 | Up |
| C2 | Down |
| C14 | Down |
| Beta-hydroxy butyric | Down |
| Spermine | Down |
| Betaine | Down |
| C14:2 | Down |
| Aspartic acid | Down |
| C18 | Down |
| | |
|---|---|
| C6 | Up |
| C3 OH | Up |
| Tryptophan | Up |
| C3:1 | Up |
| Tyrosine | Up |
| Creatinine | Up |
| LysoPC 14:0 | Up |
| C14 | Down |
| Beta-hydroxy butyric | Down |
| Betaine | Down |
| C14:2 | Down |
| Aspartic acid | Down |
| C18 | Down |

6. A method of determining a likelihood of mortality within three months in a subject having a severe traumatic brain injury (sTBI), comprising:
obtaining a sample from a subject at day 1 and/or day 4 post sTBI,
measuring an amount of a plurality of metabolites in the sample, and
comparing levels of the plurality of metabolites in the sample with a control,
wherein an outcome of the comparing step is an increase or decrease in quantity of said plurality of metabolites in the sample relative to the control, and
wherein increased likelihood of mortality is determined when:
in the day 1 sample, at least two, or at least 5, of the following metabolites of the plurality of metabolites are changed relative to the control: C3:1, PC aa 38:0, glucose, C16:2, leucine, C10:2, valine, isoleucine, histidine, C16OH, glutamine, betaine, 3-hydroxyisovalerate, citrate, and lactate;
and/or
in the day 4 sample, at least two, or at least 5, of the following metabolites of the plurality of metabolites are changed relative to the control: isobutyrate, valine, lysine, 2-aminobutyrate, hypoxanthine, taurine, gluconate, betaine, alpha-ketoglutaric acid, C16:2OH, hippuric acid, indole acetic acid, PC aa 36:0, ornithine, PC aa 38:0, alpha-aminoadipic acid, tryptophan, leucine, C12:1, C6, glutamine, and LysoPC 26:0.

7. The method of claim 6, wherein said likelihood of mortality is determined from a Day 1 sample, with increase or decrease in said at least 2, or said at least 5, of the plurality of metabolites as per Tables 43A, 43B, 45A, or 45B:

| | |
|---|---|
| C3:1 | Up |
| PC aa 38:0 | Up |
| Glucose | Up |
| PC ae 40:6 | Up |
| C10:1 | Up |
| C14:1 | Up |
| C14 | Up |
| C10 | Up |
| C16:2 | Up |
| C8 | Up |
| C12 | Up |
| Citrulline | Down |
| C10:2 | Down |
| Leucine | Down |
| Valine | Down |
| Isoleucine | Down |
| Histidine | Down |
| C16 OH | Down |
| Glutamine | Down |
| | |
|---|---|
| C3:1 | Up |
| PC aa 38:0 | Up |
| Glucose | Up |
| C16:2 | Up |
| Leucine | Down |
| C10:2 | Down |
| Valine | Down |
| Isoleucine | Down |
| Histidine | Down |
| C16 OH | Down |
| Glutamine | Down |
| | |
|---|---|
| Glucose | Up |
| Betaine | Up |
| 3-Hydroxyisovalerate | Up |
| Citrate | Up |
| O-Phosphocholine | Up |
| Dimethyl Sulfone | Up |
| Formate | Up |
| Fumarte | Up |
| 2-Oxglutarate | Up |
| Pyruvate | Up |
| Lactate | Down |
| Valine | Down |
| Isoleucine | Down |
| Leucine | Down |
| Diemthylamine | Down |
| Glutamine | Down |
| Histidine | Down |
| | |
|---|---|
| Glucose | Up |
| Betaine | Up |
| 3-Hydroxyisovalerate | Up |
| Citrate | Up |
| Lactate | Down |

8. The method of claim 6, wherein said likelihood of mortality is determined from a Day 4 sample, with an increase or decrease in said at least 2, or said at least 5, of the plurality of metabolites as per Tables 44A, 44B, 46A, 46B, or 46C:

| | |
|---|---|
| Indole acetic acid | Up |
| Alpha-Ketglutaric acid | Up |
| Hippric acid | Up |
| C16:2 OH | Up |
| Ornithine | Up |
| PC aa 36:0 | Up |
| C3 | Up |
| Threonine | Up |
| Alpha-Aminoadipic acid | Up |
| PC aa 38:0 | Up |
| Tyrosine | Up |
| Valine | Up |
| Tryptophan | Up |
| C2 | Down |
| C8 | Down |
| C12:1 | Down |
| Betaine | Down |
| C6 | Down |
| Glutamine | Down |
| Taurine | Down |
| LysoPC 26:0 | Down |
| | |
|---|---|
| Alpha-Ketoglutaric acid | Up |
| C16:2 OH | Up |
| Hippuric acid | Up |
| Indole acetic acid | Up |
| PC aa 36:0 | Up |
| Ornithine | Up |
| PC aa 38:0 | Up |
| Alpha-Aminoadipic acid | Up |
| Tryptophan | Up |
| Valine | Down |
| Leucine | Down |
| C12:1 | Down |
| C6 | Down |
| Glutamine | Down |
| LysoPC 26:0 | Down |
| Taurine | Down |
| | |
|---|---|
| Isobutyrate | Up |
| Creatine | Up |
| Creatinine | Up |
| Valine | Up |
| Lysine | Up |
| Asparagine | Up |
| Leucine | Up |
| Tyrosine | Up |
| 2-Aminobutyrate | Up |
| 4-Hydroxybutyrate | Down |
| Methionine | Down |
| Urea | Down |
| Hypoxanthine | Down |
| Taurine | Down |
| Gluconate | Down |
| Betaine | Down |
| | |
|---|---|
| Isobutyrate | Up |
| Valine | Up |
| Lysine | Up |
| 2-Aminobutyrate | Up |
| Hypoxanthine | Down |
| Taurine | Down |
| Gluconate | Down |
| Betaine | Down |
| | |
|---|---|
| Valine | Up |
| Lysine | Up |
| Taurine | Down |
| Gluconate | Down |
| Betaine | Down. |

9. A method of determining a likelihood of an outcome in a subject having a severe traumatic brain injury (sTBI) or suspected of having an sTBI, comprising:
obtaining a sample from a subject at day 1 or day 4 post-sTBI,
measuring an amount of a plurality of metabolites in the sample, and comparing levels of the plurality of metabolites in the sample with a control, wherein an outcome of the comparing step is an increase or decrease in quantity of said plurality of metabolites in the sample,
(i) wherein the likelihood of unfavourable outcome at 3 months is determined:
(a1) in a day 1 sample assessed by quantitative MS, wherein at least 2, at least 5, or at least 10 metabolites of said plurality of metabolites are increased or decreased relative to the control as indicated of the 26 metabolites in Table 35A:

| | |
|---|---|
| Lyso PC 17:0 | Up |
| Lyso PC 18:0 | Up |
| C3:1 | Up |
| Lyso PC 16:0 | Up |
| Lyso PC 18:1 | Up |
| C18:2 | Up |
| C14 | Up |
| C18 | Up |
| C18:1 | Up |
| C16 | Up |
| C14:2 | Up |
| Tyrosine | Down |
| Asparagine | Down |
| PC ae 36:0 | Down |
| C16:2 | Down |
| Phenylalanine | Down |
| C16:1 | Down |
| Glutamine | Down |
| SM 20:2 | Down |
| PC aa 32:2 | Down |
| Isoleucine | Down |
| Citrulline | Down |
| Histidine | Down |
| Glutamate | Down |
| Methionine-Sulfoxide | Down |
| Asymmetric | Down |
| dimethylargine | |

or of the 13 metabolites in Table 35B:

| | |
|---|---|
| LYSOC17:0 | Up |
| LYSOC18:0 | Up |
| LYSOC16:0 | Up |
| C18:2 | Up |
| C14 | Up |
| C18:1 | Up |
| C18 | Up |
| C16 | Up |
| Glutamine | Down |
| Histidine | Down |
| SM 20:2 | Down |
| Methionine-sulfoxide | Down |
| Asymmetric dimethylarginine | Down; |

or
(a2) in a day 4 sample assessed by MS/MS, wherein at least 2, at least 5, or at least 10 metabolites of said plurality of metabolites are increased or decreased relative to the control as indicated, of the 15 metabolites in Table 36A:

| | |
|---|---|
| C3OH | Up |
| Glutamic acid | Up |
| LYSOC18:0 | Up |
| Ornithine | Up |
| PC aa3 6:0 | Up |
| C18:2 | Up |
| alpha-Aminoadipic acid | Up |
| Indole acetic acid | Up |
| C3:1 | Up |
| PC aa 40:2 | Down |
| C16:1 | Down |
| Serine | Down |
| Glutamine | Down |
| beta-Hydroxybutyric acid | Down |
| Uric acid | Down, |

or of the 11 metabolites in Table 36B:

| | |
|---|---|
| C3OH | Up |
| Glutamic acid | Up |
| LYSOC18:0 | Up |
| Ornithine | Up |
| PC36:0AA | Up |
| C18:2 | Up |
| C3:1 | Up |
| C16:1 | Down |
| Glutamine | Down |
| beta-Hydroxybutyric acid | Down |
| Uric acid | Down; |

or
(b1) in a day 1 sample assessed by proton (1H) nuclear magnetic resonance spectroscopy (NMR), wherein at least 2, at least 5, or at least 10 metabolites of said plurality of metabolites are increased or decreased relative to the control as indicated, of the 12 metabolites in Table 39A:

| | |
|---|---|
| Ornithine | Up |
| Glucose | Up |
| Acetone | Up |
| Lactate | Up |
| Glycerol | Up |
| Betaine | Up |
| Choline | Up |
| Serine | Up |
| Glycine | Up |
| Formate | Up |
| Isoleucine | Down |
| Dimethylamine | Down, |

or at least 5 of the 6 metabolites in Table 39B:

| | |
|---|---|
| Ornithine | Up |
| Acetone | Up |
| Lactate | Up |
| Glycerol | Up |
| Betaine | Up |
| Choline | Up; |

or
(b2) in a day 4 sample assessed by NMR, wherein at least 2, or at least 5 metabolites of said plurality of metabolites are increased or decreased relative to the control as indicated, of the 9 metabolites in Table 40A:

| | |
|---|---|
| Valine | Up |
| N-Acetylaspartate | Up |
| Tyrosine | Up |
| Lysine | Up |
| Histidine | Up |
| Dimethyl Sulfone | Up |
| Pyruvate | Down |
| Taurine | Down |
| Gluoconate | Down, |

or of the 6 metabolites in Table 40B:

| | |
|---|---|
| Valine | Up |
| N-Acetylaspartate | Up |
| Tyrosine | Up |
| Lysine | Up |
| Taurine | Down |
| Gluconate | Down; |

or
(ii) wherein the likelihood of unfavourable outcome at 12 months is determined:
(c1) in a day 1 sample, assessed by QUANTITATIVE MS, wherein at least 2, at least 5, or at least 10 metabolites of said plurality of metabolites are increased or decreased relative to the control as indicated, of the 21 metabolites in Table 37A:

| | |
|---|---|
| C5OH | Down |
| Homocysteine | Up |
| C3 | Up |
| C0 | Up |
| C4 | Up |
| Ornithine | Up |
| LYSOC14:0 | Up |
| SM 16:1 OH | Up |
| LYSOC20:3 | Up |
| LYSOC28:1 | Up |
| C10:2 | Down |
| Acetyl-ornithine | Down |
| C9 | Down |
| Adimethylarginine | Down |
| Methionine-sulfoxide | Down |
| Spermine | Down |
| PC ae 36:0 | Down |
| Citrulline | Down |
| Serotonin | Down |
| Serine | Down |
| trans-Hydroxyproline | Down |

or of the 15 metabolites in Table 37B:

| | |
|---|---|
| C3 | Up |
| Ornithine | Up |
| C0 | Up |
| SM 16:1 OH | Up |
| LYSOC14:0 | Up |
| LYSOC20:3 | Up |
| Homocysteine | Up |
| Serotonin | Down |
| C9 | Down |
| PC ae 36:0 | Down |
| Methionine-sulfoxide | Down |
| Serine | Down |
| Adimethylarginine | Down |
| Spermine | Down |
| trans-Hydroxyproline | Down; |

or
(c2) in a day 4 sample, assessed by quantitative MS, wherein at least 2, at least 5, or at least 10 metabolites of said plurality of metabolites are increased or decreased relative to the control as indicated, of the 18 metabolites in Table 38A:

| | |
|---|---|
| C6 | Up |
| C3 OH | Up |
| C18:1 OH | Up |
| Tryptophan | Up |
| C3:1 | Up |
| Tyrosine | Up |
| Creatinine | Up |
| LysoPC 14:0 | Up |
| Alanine | Up |
| C16 | Up |
| C2 | Down |
| C14 | Down |
| Beta-hydroxy butyric | Down |
| Spermine | Down |
| Betaine | Down |
| C14:2 | Down |
| Aspartic acid | Down |
| C18 | Down |

or of the 13 metabolites in Table 38B:

| | |
|---|---|
| C6 | Up |
| C3 OH | Up |
| Tryptophan | Up |
| C3:1 | Up |
| Tyrosine | Up |
| Creatinine | Up |
| LysoPC 14:0 | Up |
| C14 | Down |
| Beta-hydroxy butyric | Down |
| Betaine | Down |
| C14:2 | Down |
| Aspartic acid | Down |
| C18 | Down; |

or
(d1) in a day 1 sample assessed by NMR, wherein at least 2, or at least 5 metabolites of said plurality of metabolites are increased or decreased relative to the control as indicated, of the 8 metabolites in Table 41A:

| | |
|---|---|
| Ornithine | Up |
| Valine | Up |
| Succinate | Up |
| Leucine | Up |
| Gluconate | Up |
| Alanine | Up |
| Mannose | Down |
| 3-Hyroxyisovalerate | Down, |

or at least 4 of the 5 metabolites in Table 41B:

| | |
|---|---|
| Ornithine | Up |
| Succinate | Up |
| Gluconate | Up |
| Alanine | Down |
| 3-Hyroxyisovalerate | Down; |

or
(d2) in a day 4 sample assessed by NMR, wherein at least 2, or at least 5 metabolites of said plurality of metabolites are increased or decreased relative to the control as indicated, of the 9 metabolites in Table 42A:

| | |
|---|---|
| Dimethyl sulfone | Up |
| Tyrosine | Up |
| Hisitidine | Up |
| Valine | Up |
| Leucine | Up |
| Taurine | Up |
| Hypoxanthine | Down |
| Isopropanol | Down |
| Beta-alanine | Down, |

or of the 5 metabolites in Table 42B:

| | |
|---|---|
| Dimethyl sulfone | Up |
| Tyrosine | Up |
| Valine | Up |
| Hypoxanthine | Down |
| Beta-alanine | Down; |

or
(iii) wherein the likelihood of mortality outcome is determined:
(e1) in a day 1 sample, assessed by quantitative MS, wherein at least 2, at least 5, or at least 10 metabolites of said plurality of metabolites are increased or decreased relative to the control as indicated, of the 19 metabolites in Table 43A:

| | |
|---|---|
| C3:1 | Up |
| PC aa 38:0 | Up |
| Glucose | Up |
| PC ae 40:6 | Up |
| C10:1 | Up |
| C14:1 | Up |
| C14 | Up |
| C10 | Up |
| C16:2 | Up |
| C8 | Up |
| C12 | Up |
| Citrulline | Down |
| C10:2 | Down |
| Leucine | Down |
| Valine | Down |
| Isoleucine | Down |
| Histidine | Down |
| C16 OH | Down |
| Glutamine | Down |

or of the 11 metabolites in Table 43B;

| | |
|---|---|
| C3:1 | Up |
| PC aa 38:0 | Up |
| Glucose | Up |
| C16:2 | Up |
| Leucine | Down |
| C10:2 | Down |
| Valine | Down |
| Isoleucine | Down |
| Histidine | Down |
| C16 OH | Down |
| Glutamine | Down; |

or
(e2) in a day 4 sample, assessed by quantitative MS, wherein at least 2, at least 5, or at least 10 metabolites of said plurality of metabolites are increased or decreased relative to the control as indicated, of the 22 metabolites in Table 44A:

| | |
|---|---|
| Indole acetic acid | Up |
| Alpha-Ketglutaric acid | Up |
| Hippric acid | Up |
| C16:2 OH | Up |
| Ornithine | Up |
| PC aa 36:0 | Up |
| C3 | Up |
| Threonine | Up |
| Alpha-Aminoadipic acid | Up |
| PC aa 38:0 | Up |
| Tyrosine | Up |
| Valine | Up |
| Tryptophan | Up |
| C2 | Down |
| C8 | Down |
| C12:1 | Down |
| Betaine | Down |
| C6 | Down |
| Glutamine | Down |
| Taurine | Down |
| LysoPC 26:0 | Down, |

or of the 16 metabolites in Table 44B:

| | |
|---|---|
| Alpha-Ketoglutaric acid | Up |
| C16:2 OH | Up |
| Hippuric acid | Up |
| Indole acetic acid | Up |
| PC aa 36:0 | Up |
| Ornithine | Up |
| PC aa 38:0 | Up |
| Alpha-Aminoadipic acid | Up |
| Tryptophan | Up |
| Valine | Down |
| Leucine | Down |
| C12:1 | Down |
| C6 | Down |
| Glutamine | Down |
| LysoPC 26:0 | Down |
| Taurine | Down; |

or
(f1) in a day 1 sample, assessed by NMR, wherein at least 2, at least 5, or at least 10 metabolites of said plurality of metabolites are increased or decreased relative to the control as indicated, of the 17 metabolites in Table 45A:

| | |
|---|---|
| Glucose | Up |
| Betaine | Up |
| 3-Hydroxyisovalerate | Up |
| Citrate | Up |
| O-Phosphocholine | Up |
| Dimethyl Sulfone | Up |
| Formate | Up |
| Fumarte | Up |
| 2-Oxglutarate | Up |
| Pyruvate | Up |
| Lactate | Down |
| Valine | Down |
| Isoleucine | Down |
| Leucine | Down |
| Diemthylamine | Down |
| Glutamine | Down |
| Histidine | Down, |

or at least 4 of the 5 metabolites in Table 45B:

| | |
|---|---|
| Glucose | Up |
| Betaine | Up |
| 3-Hydroxyisovalerate | Up |
| Citrate | Up |
| Lactate | Down; |

or
(f2) in a day 4 sample, assessed by NMR, wherein at least 2, or at least 5 metabolites of said plurality of metabolites are increased or decreased relative to the control as indicated, of the 16 metabolites in Table 46A:

| | |
|---|---|
| Isobutyrate | Up |
| Creatine | Up |
| Creatinine | Up |
| Valine | Up |
| Lysine | Up |
| Asparagine | Up |
| Leucine | Up |
| Tyrosine | Up |
| 2-Aminobutyrate | Up |
| 4-Hydroxybutyrate | Down |
| Methionine | Down |
| Urea | Down |
| Hypoxanthine | Down |
| Taurine | Down |
| Gluconate | Down |
| Betaine | Down, |

of the 8 metabolites in Table 46B:

| | |
|---|---|
| Isobutyrate | Up |
| Valine | Up |
| Lysine | Up |
| 2-Aminobutyrate | Up |
| Hypoxanthine | Down |
| Taurine | Down |
| Gluconate | Down |
| Betaine | Down, |

or at least 2 or 4 of the 5 metabolites in Table 46C:

| | |
|---|---|
| Valine | Up |
| Lysine | Up |
| Taurine | Down |
| Gluconate | Down |
| Betaine | Down. |

10. The method of claim 9, wherein said likelihood of said unfavorable outcome is predicted for three months following the sTBI.

11. The method of claim 9, wherein said likelihood of said unfavorable outcome is predicted for twelve months following the sTBI.

12. The method of claim 9, wherein said likelihood of mortality is predicted.

13. The method of any one of claims 1 to 12, wherein sample is a serum sample.

14. The method of any one of claims 1 to 13, wherein the control is a value determined from individuals with an orthopedic injury (OI) without head injury, or from a cohort of individuals with sTBI having a favorable outcome.

15. The method of any one of claims 1 to 14, wherein said amount of said plurality of metabolites are assessed by NMR.

16. The method of any one of claims 1 to 14, wherein said amount of said plurality of metabolites are assessed by quantitative MS.

17. The method of any one of claims 1 to 16, wherein said sample is obtained at day 1 following the sTBI.

18. The method of any one of claims 1 to 16, wherein said sample is obtained at day 4 following the sTBI.

19. The method of any one of claims 1 to 12, wherein said sample is obtained at day 1 and day 4 following the sTBI.

20. The method of claim 9, wherein the likelihood of unfavourable outcome at 3 months is determined by assessing the increase or decrease in the following metabolites:
(a1) in a day 1 sample, assessed by quantitative MS wherein the metabolites are increased or decreased as indicated in Table 35A or Table 35B;
(a2) in a day 4 sample, assessed by quantitative MS wherein the metabolites are increased or decreased as indicated in Table 36A or Table 36B;
(b1) in a day 1 sample, assessed by NMR wherein the metabolites are increased or decreased as indicated in Table 39A or Table 39B; or
(b2) in a day 4 sample, assessed by NMR wherein the metabolites are increased or decreased as indicated in Table 40A or Table 40B.

21. The method of claim 9, wherein the likelihood of unfavourable outcome at 12 months is determined by assessing the increase or decrease in the following metabolites:
(c1) in a day 1 sample, assessed by quantitative MS wherein the metabolites are increased or decreased as indicated in Table 37A or Table 37B;
(c2) in a day 4 sample, assessed by quantitative MS wherein the metabolites are increased or decreased as indicated in Table 38A or Table 38B;
(d1) in a day 1 sample, assessed by NMR wherein the metabolites are increased or decreased as indicated in Table 41A or Table 41B; or
(d2) in a day 4 sample, assessed by NMR wherein the metabolites are increased or decreased as indicated in Table 42A or Table 42B.

22. The method of claim 9, wherein the likelihood of mortality outcome at 3 months is determined by assessing the increase or decrease in the following metabolites:
(e1) in a day 1 sample, assessed by quantitative MS wherein the metabolites are increased or decreased as indicated in Table 43A or Table 43B;
(e2) in a day 4 sample, assessed by quantitative MS wherein the metabolites are increased or decreased as indicated in Table 44A or Table 44B;
(f1) in a day 1 sample, assessed by NMR wherein the metabolites are increased or decreased as indicated in Table 45A or Table 45B; or
(f2) in a day 4 sample, assessed by NMR wherein the metabolites are increased or decreased as indicated in Table 46A, Table 46B, or Table 46C.

23. The method of claim 9, wherein the likelihood of mortality outcome at 3 months is determined by assessing the increase or decrease in metabolites:
(e1) in a day 1 sample, assessed by quantitative MS wherein the metabolites are increased or decreased as indicated in Table 43B;
(e2) in a day 4 sample, assessed by quantitative MS wherein the metabolites are increased or decreased as indicated in Table 44B;
(f1) in a day 1 sample, assessed by NMR wherein the metabolites are increased or decreased as indicated in Table 45B; or
(f2) in a day 4 sample, assessed by NMR wherein the metabolites are increased or decreased as indicated in Table 46C.

24. A kit for predicting outcome of a traumatic brain injury in a subject comprising:
reagents for detecting the metabolites listed in any one of Tables 35A, 35B, 36A, 36B, 37A, 37B, 38A, 38B, 39A, 39B, 40A, 40B, 41A, 41B, 42A, 42B, 43A, 43B, 44A, 44B, 45A, 45B, 46A, 46B, or 46C, and
instructions for conducting the method of any one of claims 1 to 23.
